(19) United States
(12) Patent Application Publication
(10) Pub. No.: US 2024/0182898 A1
(43) Pub. Date: Jun. 6, 2024

(54) ENHANCED OLIGONUCLEOTIDES FOR INHIBITING RTELl EXPRESSION

(71) Applicant: HOFFMANN-LA ROCHE INC., Little Falls, NJ (US)

(72) Inventors: Erik FUNDER, Hørsholm (DK); Natascha HRUSCHKA, Basel (CH); Susanne KAMMLER, Hørsholm (DK); Erich Koller, Basel (CH); Brian Leonard, Basel (CH); Souphalone LUANGSAY, Basel (CH); Susanne MOHR, Basel (CH); Tobias NILSSON, Basel (CH); Søren OTTOSEN, Hørsholm (DK); Lykke PEDERSEN, Hørsholm (DK); Søren RASMUSSEN, Hørsholm (DK); Steffan SCHMIDT, Hørsholm (DK); Sabine SEWING, Basel (CH); Daniel Turley, Basel (CH); Johanna Marie WALTHER, Basel (CH)

(21) Appl. No.: 18/540,306

(22) Filed: Dec. 14, 2023

Related U.S. Application Data
(63) Continuation of application No. 17/590,754, filed on Feb. 1, 2022, now Pat. No. 11,898,145.

(30) Foreign Application Priority Data
Feb. 2, 2021 (EP) 21154701.3
Nov. 8, 2021 (EP) 21207002.3

(51) Int. Cl. C12N 15/113; A61K 31/7088; A61P 31/20

(52) U.S. Cl. CPC C12N 15/113; A61K 31/7088; A61P 31/20; C12N 2310/11; C12N 2310/315; C12N 2310/3231; C12N 2310/341; C12N 2310/351

(57) ABSTRACT

Enhanced antisense oligonucleotides targeting Regulator of telomere elongation helicase 1 (RTEL1), leading to modulation of the expression of RTEL1 or modulation of RTEL1 activity are provided. This disclosure relates to the use of enhanced antisense oligonucleotides targeting RTEL1 for use in treating and/or preventing a hepatitis B virus (HBV) infection, in particular a chronic HBV infection. This disclosure further relates to the use of the enhanced antisense oligonucleotides targeting RTEL1 for destabilizing cccDNA, such as HBV cccDNA. A pharmaceutical composition and its use in the treatment and/or prevention of a HBV infection is also described.

### CROSS-REFERENCE TO RELATED APPLICATIONS

This application claims priority to European Application No. 21154701.3 filed 2 Feb. 2021 and European Application No. 21207002.3 filed 8 Nov. 2021, the disclosures of which are incorporated herein, in their entireties, by this reference.

### REFERENCE TO A SEQUENCE LISTING SUBMITTED VIA EFS-WEB

The content of the ASCII text file of the sequence listing named “RD35909US_SEQ_LISTING.TXT” which was created on Feb. 1, 2022, and is 112,314 bytes in size, submitted electronically via EFS-Web in this U.S. non-provisional patent application is incorporated by reference in its entirety.

### FIELD OF INVENTION

The present invention relates to enhanced antisense oligonucleotides targeting RTEL1 (Regulator of telomere elongation helicase 1), leading to modulation of the expression of RTEL1 or modulation of RTEL1 activity. The invention, in particular, relates to the use of enhanced antisense oligonucleotides targeting RTEL1 for use in treating and/or preventing a hepatitis B virus (HBV) infection, in particular a chronic HBV infection. The invention, in particular, relates to the use of the enhanced antisense oligonucleotides targeting RTEL1 for destabilizing cccDNA, such as HBV cccDNA. Also comprised in the present invention is a pharmaceutical composition and its use in the treatment and/or prevention of an HBV infection.

### BACKGROUND

Hepatitis B is an infectious disease caused by the hepatitis B virus (HBV), a small hepatotropic virus that replicates through reverse transcription. Chronic HBV infection is a key factor for severe liver diseases such as liver cirrhosis and hepatocellular carcinoma. Current treatments for chronic HBV infection are based on administration of pegylated type 1 interferons or nucleos(t)ide analogues, such as lamivudine, adefovir, entecavir, tenofovir disoproxil, and tenofovir alafenamide, which target the viral polymerase, a multifunctional reverse transcriptase. Treatment success is usually measured as loss of hepatitis B surface antigen (HBsAg). However, a complete HBsAg clearance is rarely achieved since Hepatitis B virus DNA persists in the body after infection. HBV persistence is mediated by an episomal form of the HBV genome which is stably maintained in the nucleus. This episomal form is called “covalently closed circular DNA” (cccDNA). The cccDNA serves as a template for all HBV transcripts, including pregenomic RNA (pgRNA), a viral replicative intermediate. The presence of a few copies of cccDNA might be sufficient to reinitiate a HBV infection. Current treatments for HBV do not target cccDNA. A cure of chronic HBV infection, however, would require the elimination of cccDNA (reviewed by Nassal, Gut. 2015 December; 64(12):1972-84. doi: 10.1136/gutjnl-2015-309809).

Regulator of telomere elongation helicase 1 (RTEL1) encodes a DNA helicase which functions in the stability, protection and elongation of telomeres and interacts with proteins in the sheltering complex known to protect telomeres during DNA replication. Mutations in this gene have been associated with dyskeratosis congenita and Hoyerall-Hreidarsson syndrome (See for example review by Vannier et al., 2014 Trends Cell Biol. Vol 24 p. 416).

Located in the nucleus, RTEL1 functions as an ATP-dependent DNA helicase implicated in telomere-length regulation, DNA repair and the maintenance of genomic stability. RTEL1 acts as an anti-recombinase to counteract toxic recombination and limit crossover during meiosis and regulates meiotic recombination and crossover homeostasis by physically dissociating strand invasion events and thereby promotes non-crossover repair by meiotic synthesis dependent strand annealing (SDSA) as well as disassembly of D loop recombination intermediates. In addition, RTEL1 disassembles T-loops and prevents telomere fragility by counteracting telomeric G4-DNA structures, which together ensure the dynamics and stability of the telomere.

RTEL1 has been identified in a siRNA screen as a stabilizer of HPV episomes: (Edwards et al., 2013 PLoS One Vol 8, e75406). siRNA targeting RTEL1 has likewise been used to identify interactants with RTEL1 in Hoyeraal-Hreidarsson syndrome (Schertzer et al 2015 Nucleic Acid Res Vol 43 p. 1834). In addition, RTEL1 was identified as a HIV host dependency factor from a siRNA screen for essential host proteins to provide targets for inhibition HIV infection (WO 2007/094818).

In WO 2020/011902, it was shown that targeting RTEL1 with antisense oligonucleotides, resulted in reduction of RTEL1. Further, an effect on parameters of HBV infection, such as HBV cccDNA, pgRNA, HBsAg, and HBeAg, was observed.

There is a need for therapeutic agents, which can inhibit RTEL1 specifically. We have screened more than 1000 antisense oligonucleotides targeting human RTEL1 and identified sequences and compounds, which are particularly potent and effective to specifically target for human RTEL1.

Specifically, four LNA gapmer oligonucleotides were identified which conferred a strong down-regulation of human RTEL1 in vitro. The identified LNA gapmer oligonucleotides target intronic regions of the human RTEL1 pre-mRNA.

### OBJECTIVE OF THE INVENTION

The present invention provides antisense oligonucleotides and conjugates thereof which modulate RTEL1 (“Regulator of telomere elongation helicase 1”) expression. We identified intronic target sequences present of the human RTEL1 pre-mRNA which may be targeted by antisense oligonucleotides, or conjugates thereof, to give effective RTEL1 inhibition. For example, targeting position 8681-8701 or 11753-11774 of SEQ ID NO: 1 is advantageous in terms of reducing RTEL1. Accordingly, an objective of the present invention is to provide enhanced antisense oligonucleotides targeting RTEL1, or conjugates thereof, wherein the antisense oligonucleotides are capable of inhibiting the expression of RTEL1 in vitro and in vivo, thereby reducing cccDNA in an HBV infected cell. The enhanced antisense oligonucleotides targeting RTEL1 or the conjugate thereof can be used in the treatment of HBV infection.

### SUMMARY OF INVENTION

The present invention provides antisense oligonucleotides or conjugates thereof, which are complementary to, and are capable of inhibiting the expression of a RTEL1 nucleic acid, and for their use in medicine.

The invention provides for an antisense oligonucleotide which comprises a contiguous nucleotide sequence which is complementary to, such as fully complementary to a region of the human RTEL1 pre-mRNA (as illustrated in SEQ ID NO: 1), such as a region from nucleotides 8681-8701 or from nucleotides 11753-11774 of SEQ ID NO: 1.

In some embodiments, the antisense oligonucleotide or the contiguous nucleotide sequence is complementary to, such as fully complementary to a region from nucleotides 8681-8701 of SEQ ID NO: 1.

In some embodiments, the antisense oligonucleotide or the contiguous nucleotide sequence is complementary to, such as fully complementary to a region from nucleotides 11753-11774 of SEQ ID NO: 1, such as to a region from nucleotides 11757-11774, 11756-11774, or 11753-11770 of SEQ ID NO: 1.

The antisense oligonucleotide of the invention is typically 12-24 nucleotides in length, such as 12 to 22, such as 16 to 22 nucleotides in length, and comprises a contiguous nucleotide sequence of at least 12 nucleotides which is complementary to, such as fully complementary to a region of the human RTEL1 pre-mRNA (as illustrated in SEQ ID NO: 1), selected from nucleotides 8681-8701 and 11753-11774 of SEQ ID NO: 1.

The invention provides for an antisense oligonucleotide, 12-22 nucleotides in length, wherein said antisense oligonucleotide comprises a contiguous nucleotide sequence 12-22 nucleotides in length, wherein the contiguous nucleotide sequence is complementary, such as fully complementary, to SEQ ID NO 7, 8, 9 and/or 10.

The invention provides for an antisense oligonucleotide, 12-22 nucleotides in length (such as 15, 16, 17, 18, 19 or 20 nucleotides in length), wherein said antisense oligonucleotide comprises a contiguous nucleotide sequence 12-18 nucleotides in length (such as 15, 16, 17, or 18 nucleotides in length), wherein the contiguous nucleotide sequence is complementary, such as fully complementary, to SEQ ID NO 11.

The invention provides for an antisense oligonucleotide 10 to 30 nucleotides in length, which comprises a contiguous nucleotide sequence of 10 to 30 nucleotides in length, wherein the contiguous nucleotide sequence is 100% identical to a sequence selected from the group consisting of SEQ ID NOs: 3, 4, 5 and 6; or at least 14 contiguous nucleotides thereof.

The invention provides for an antisense oligonucleotide 10 to 30 nucleotides in length, which comprises a contiguous nucleotide sequence of 10 to 30 nucleotides in length, wherein the contiguous nucleotide sequence is 100% identical to a sequence selected from the group consisting of SEQ ID NOs: 3, 4, 5 and 6, or at least 15 contiguous nucleotides thereof.

The invention provides for an antisense oligonucleotide 10 to 30 nucleotides in length, which comprises a contiguous nucleotide sequence of 10 to 30 nucleotides in length, wherein the contiguous nucleotide sequence is 100% identical to a sequence selected from the group consisting of SEQ ID NOs: 3, 4, 5 and 6, or at least 16 contiguous nucleotides thereof.

The invention provides for an antisense oligonucleotide, which comprises a contiguous nucleotide sequence, which is 100% identical to a sequence selected from the group consisting of SEQ ID NOs: 3, 4, 5 and 6, or at least 14 contiguous nucleotides thereof.

The invention provides for an antisense oligonucleotide, which comprises a contiguous nucleotide sequence selected from the group consisting of SEQ ID NOs: 3, 4, 5 and 6.

The invention provides for an antisense oligonucleotide, which comprises a contiguous nucleotide sequence, which is 100% identical to SEQ ID NO: 3 (AATTTTACATACTCTGGT), or at least 14, 15, 16 or 17 contiguous nucleotides thereof.

The invention provides for an antisense oligonucleotide, which comprises a contiguous nucleotide sequence, which is 100% identical to SEQ ID NO: 4 (AATTTTACATACTCTGGTC), or at least 14, 15, 16, 17 or 18 contiguous nucleotides thereof.

The invention provides for an antisense oligonucleotide, which comprises a contiguous nucleotide sequence, which is 100% identical to SEQ ID NO: 5 (TTACATACTCTGGTCAAA), or at least 14, 15, 16, or 17 contiguous nucleotides thereof.

The invention provides for an antisense oligonucleotide, which comprises a contiguous nucleotide sequence, which is 100% identical to SEQ ID NO: 6 (CTTTATTATAACTTGAATCTC), or at least 14, 15, 16, 17, 18, 19, 20 or 21 contiguous nucleotides thereof.

The invention provides for an antisense oligonucleotide, which comprises the contiguous nucleotide of a compound selected from the group consisting of compound ID Nos 3_1, 4_1, 5_1 and 6_1 (see e.g. Table 6 below).

In some embodiments, the antisense oligonucleotide is not an antisense oligonucleotide selected from the group consisting of compound ID Nos 36_1, 35_1. 33_1, 34_1 and 37_1 of WO 2020/011902 A1 (see e.g. Table 6 of WO 2020/011902 A1).

In some embodiments, the antisense oligonucleotide is not an antisense oligonucleotide compound ID Nos 23_1 of WO 2020/011902 A1 (see e.g. Table 6 of WO 2020/011902 A1).

The invention provides for an antisense oligonucleotide selected from the group listed in Table 1, or a pharmaceutically acceptable salt thereof.

The invention provides for an antisense oligonucleotide selected from the group consisting of
AATTttacatactctgGT (SEQ ID NO: 3, Compound ID No 3_1),
AAttttacatactctGGTC (SEQ ID NO: 4, Compound ID No 4_1),
TTacatactctggtCAAA (SEQ ID NO: 5, Compound ID No 5_1), and
CTttattataactTgaAtCTC (SEQ ID NO: 6, Compound ID No 6_1),
wherein capital letters are beta-D-oxy LNA nucleosides, lowercase letters are DNA nucleosides, all LNA Cs are LNA 5-methyl cytosine, and all internucleoside linkages are phosphorothioate internucleoside linkages.

The present invention also provides for a pharmaceutically acceptable salt of the antisense oligonucleotide of the present invention.

The invention provides for an antisense oligonucleotide selected from the group listed in Table 1, or a pharmaceutically acceptable salt thereof.

TABLE 1
Compound Table (Exemplary antisense oligonucleotides of the present invention)—HELM
Annotation Format

| | | | Comprised |
|---|---|---|---|
| | Compound | | by conjugate |
| SEQ ID | ID | HELM Annotation | shown in |
| Number | Number # | Written 5′-3′ | FIG. |
| 3 | 3_1 | [LR](A)[sP].[LR](A)[sP].[LR](T)[sP].[LR](T)[sP].[dR](T)[sP].[dR] | 1 |
| | | (T)[sP].[dR](A)[sP].[dR](C)[sP].[dR](A)[sP].[dR](T)[sP].[dR](A) | |
| | | [sP].[dR](C)[sP].[dR](T)[sP].[dR](C)[sP].[dR](T)[sP].[dR](G)[sP]. | |
| | | [LR](G)[sP].[LR](T) | |
| 4 | 4_1 | [LR](A)[sP].[LR](A)[sP].[dR](T)[sP].[dR](T)[sP].[dR](T)[sP].[dR] | 2 |
| | | (T)[sP].[dR](A)[sP].[dR](C)[sP].[dR](A)[sP].[dR](T)[sP].[dR](A) | |
| | | [sP].[dR](C)[sP].[dR](T)[sP].[dR](C)[sP].[dR](T)[sP].[LR](G)[sP]. | |
| | | [LR](G)[sP].[LR](T)[sP].[LR]([5meC]) | |
| 5 | 5_1 | [LR](T)[sP].[LR](T)[sP].[dR](A)[sP].[dR](C)[sP].[dR](A)[sP].[dR] | 4 |
| | | (T)[sP].[dR](A)[sP].[dR](C)[sP].[dR](T)[sP].[dR](C)[sP].[dR](T) | |
| | | [sP].[dR](G)[sP].[dR](G)[sP].[dR](T)[sP].[LR]([5meC])[sP].[LR] | |
| | | (A)[sP].[LR](A)[sP].[LR](A) | |
| 6 | 6_1 | [LR]([5meC])[sP].[LR](T)[sP].[dR](T)[sP].[dR](T)[sP].[dR](A)[sP]. | 3 |
| | | [dR](T)[sP].[dR](T)[sP].[dR](A)[sP].[dR](T)[sP].[dR](A)[sP].[dR] | |
| | | (A)[sP].[dR](C)[sP].[dR](T)[sP].[LR](T)[sP].[dR](G)[sP].[dR](A) | |
| | | [sP].[LR](A)[sP].[dR](T)[sP].[LR]([5meC])[sP].[LR](T)[sP].[LR] | |
| | | ([5meC]) | |

Helm Annotation Key:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC]) is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR](C) is a DNA cytosine nucleoside,
[sP]is a phosphorothioate internucleoside linkage,
P is a phosphodiester internucleoside linkage.

The invention thus provides for an antisense oligonucleotide selected from the group consisting of compound ID Nos #3_1, 4_1, 5_1 and 6_1.

The present invention further provides a conjugate comprising the antisense oligonucleotide of the present invention and at least one conjugate moiety covalently attached to said antisense oligonucleotide.

In some embodiments, the conjugate moiety is capable of binding to the asialoglycoprotein receptor, such as the human asialoglycoprotein receptor. For example, the conjugate moiety may comprise at least one asialoglycoprotein receptor targeting moiety selected from the group consisting of galactose, galactosamine, N-formyl-galactosamine, N-acetylgalactosamine, N-propionyl-galactosamine, N-n-butanoyl-galactosamine and N-isobutanoylgalactosamine.

In some embodiments, the asialoglycoprotein receptor-targeting moiety is N-acetylgalactosamine (GaINAc). Thus, the antisense oligonucleotide of the present invention may be conjugated to at least one conjugate moiety comprising at least one N-Acetylgalactosamine (GaINAc) moiety, such as at least one conjugate moiety comprising at least one N-Acetylgalactosamine (GaINAc) moiety as described below. According to one aspect of the invention, the conjugate moiety is a GaINAc residue R as described hereinunder.

In some embodiments, the conjugate moiety is an at least trivalent, such as a divalent, trivalent or tetravalent GaINAc residue R. Preferably the conjugate moiety is a trivalent GaINAc residue R. The term “trivalent GaINAc residue” as used herein refers to a residue comprising three N-Acetylgalactosamine moieties, i.e. preferably three moieties of formula

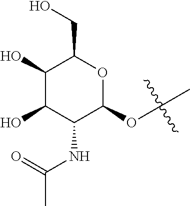 [SMILES: *O[C@@H]1O[C@H](CO)[C@H](O)[C@H](O)[C@H]1NC(C)=O]

The conjugate moiety or the GaINAc residue R, respectively, and the antisense oligonucleotide may be linked together via a linker L, such as a biocleavable linker L. Thus, the conjugate compound may comprise a linker L, which is positioned between the antisense oligonucleotide and the conjugate moiety or GaINAc residue R, respectively.

In one embodiment, the conjugate moiety is a tri-valent N-acetylgalactosamine (GaINAc), such as those shown in FIG. 5. In one embodiment, the conjugate moiety is the tri-valent N-acetylgalactosamine (GaINAc) of FIG. 5A-1 or FIG. 5A-2, or a mixture of both. In one embodiment, the conjugate moiety is the tri-valent N-acetylgalactosamine (GaINAc) of FIG. 5B-1 or FIG. 5B-2, or a mixture of both. In one embodiment, the conjugate moiety is the tri-valent N-acetylgalactosamine (GaINAc) of FIG. 5C-1 or FIG. 5C-2, or a mixture of both. In one embodiment, the conjugate moiety is the tri-valent N-acetylgalactosamine (GaINAc) of FIG. 5D-1 or FIG. 5D-2, or a mixture of both. The conjugate moiety and the antisense oligonucleotide may be linked together via a linker, such as a biocleavable linker. Thus, the conjugate compound may comprise a linker, which is positioned between the antisense oligonucleotide and the conjugate moiety.

In some embodiments, the linker comprises or consists of 1 to 10 linked nucleosides, such as 1, 2, 3, 4, 5, 6, 7, 8, 9 or 10 linked nucleosides, such as between 2 and 6 linked nucleosides, such as between 2 and 5 linked nucleosides, such as between 2 and 4 linked nucleosides. In some embodiments, the linker comprises two linked nucleotides. Thus, the nucleosides may be DNA nucleosides. Typically, the nucleosides are linked via phosphodiester internucleoside linkages. Moreover, the linker may be linked to the antisense compound via a phosphodiester internucleoside linkage.

Exemplary conjugates are provided in Table 2 (in HELM Annotation format). In addition to the compounds shown in Table 1, the compound comprise two nucleotides “ca” as cleavable linker at the 5′ end. This is part of the HELM annotation of the sequence in table 2. The cleavable linker is cleaved off after successful GaINAc mediated delivery, leaving the compound as active drug.

The invention provides for a conjugate selected from the group of conjugates listed in Table 2, or a pharmaceutically acceptable salt thereof.

TABLE 2
Compound Table (Exemplary conjugates of the present invention)—HELM Annotation
Format (for the annotation on the HELM annotation, see explanations for Table 1).

| | Oligo | | |
|---|---|---|---|
| | Compound | | |
| | ID | | |
| | Number # | | |
| | (acc. to | | Exemplary |
| SEQ ID | Table 1 | HELM Annotation | compound— |
| Number | above) | Written 5′-3′. | see FIG. |
| 3 | 3_1_Gal | {[5gn2c6]P.[dR](C)P.[dR](A)P.[LR](A)[sP].[LR](A)[sP].[LR](T) | 1 |
| | NAc | [sP].[LR](T)[sP].[dR](T)[sP].[dR](T)[sP].[dR](A)[sP].[dR](C)[sP]. | |
| | | [dR](A)[sP].[dR](T)[sP].[dR](A)[sP].[dR](C)[sP].[dR](T)[sP].[dR] | |
| | | (C)[sP].[dR](T)[sP].[dR](G)[sP].[LR](G)[sP].[LR](T) | |
| 4 | 4_1_Gal | {[5gn2c6]P.[dR](C)P.[dR](A)P.[LR](A)[sP].[LR](A)[sP].[d R](T) | 2 |
| | NAc | [sP].[dR](T)[sP].[dR](T)[sP].[dR](T)[sP].[dR](A)[sP].[dR](C)[sP]. | |
| | | [dR](A)[sP].[dR](T)[sP].[dR](A)[sP].[dR](C)[sP].[dR](T)[sP].[dR] | |
| | | (C)[sP].[dR](T)[sP].[LR](G)[sP].[LR](G)[sP].[LR](T)[sP].[LR] | |
| | | ([5meCD} | |
| 5 | 5_1_Gal | [5gn2c6]P.[dR](C)P.[dR](A)P.[LR](T)[sP].[LR](T)[sP].[dR](A) | 4 |
| | NAc | [sP].[dR](C)[sP].[dR](A)[sP].[dR](T)[sP].[dR](A)[sP].[dR](C) | |
| | | [sP].[dR](T)[sP].[dR](C)[sP].[dR](T)[sP].[dR](G)[sP].[dR](G) | |
| | | [sP].[dR](T)[sP].[LR]([5meC])[sP].[LR](A)[sP].[LR](A)[sP]. | |
| | | [LR](A) | |
| 6 | 6_1_Gal | {[5gn2c6]P.[dR](C)P.[dR](A)P.[LR]([5meC])[sP].[LR](T)[sP]. | 3 |
| | NAc | [dR](T)[sP].[dR](T)[sP].[dR](A)[sP].[dR](T)[sP].[dR](T)[sP].[dR] | |
| | | (A)[sP].[dR](T)[sP].[dR](A)[sP].[dR](A)[sP].[dR](C)[sP].[dR](T) | |
| | | [sP].[LR](T)[sP].[dR](G)[sP].[dR](A)[sP].[LR](A)[sP].[dR](T) | |
| | | [sP].[LR]([5meC])[sP].[LR](T)[sP].[LR]([5meC]) | |

In the above table, 5gn2c6 is a trivalent N-acetylgalactosamine (GaINAc) of FIG. 5D-1 or FIG. 5D-2, or a mixture of both. In some embodiments, 5gn2c6 is a GaINAc residue R having the formula:

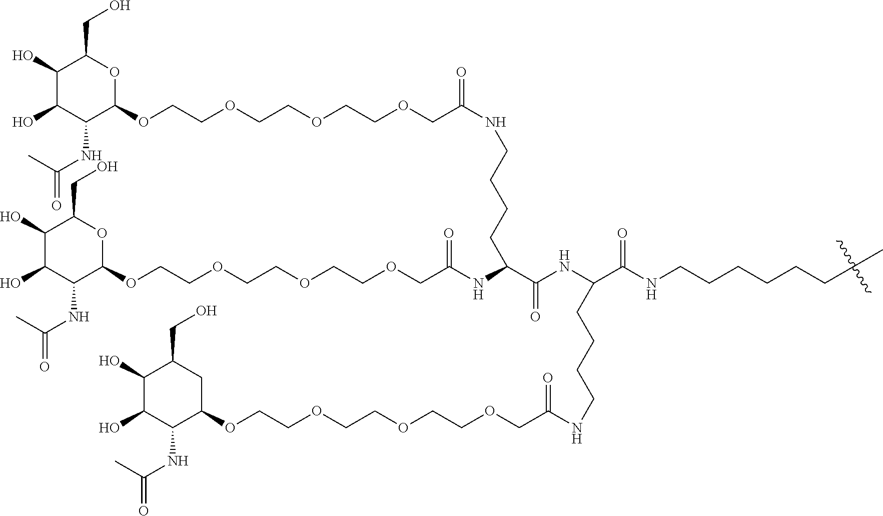 [SMILES: *CCCCCCNC(=O)C(CCCCNC(=O)COCCOCCOCCO[C@@H]1C[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

It is to be understood that R as shown in the figure above is a mixture of the two stereoisomers shown in FIGS. 5D1 and 5D2.

According to a further aspect of the invention, R as shown in the figure above is the stereoisomer as shown in to FIG. 5D1.

According to a further aspect of the invention R as shown in the figure above is the stereoisomer as shown in FIG. 5D2. The structures of the conjugates provided in Table 2 are shown in FIGS. 1 to 4.

The invention provides for the conjugate of FIG. 1, or a pharmaceutically acceptable salt thereof. The invention provides for the antisense oligonucleotide of Compound ID Number 3_1, or a pharmaceutically acceptable salt thereof.

The invention provides for the conjugate of FIG. 2, or a pharmaceutically acceptable salt thereof. The invention provides for the antisense oligonucleotide of Compound ID Number 4_1, or a pharmaceutically acceptable salt thereof.

The invention provides for the conjugate of FIG. 3, or a pharmaceutically acceptable salt thereof. The invention provides for the antisense oligonucleotide of Compound ID Number 6_1, or a pharmaceutically acceptable salt thereof.

The invention provides for the conjugate of FIG. 4, or a pharmaceutically acceptable salt thereof. The invention provides for the antisense oligonucleotide of Compound ID Number 5_1, or a pharmaceutically acceptable salt thereof.

### The Compound of Formula (I)

The present invention also provides for compounds of the following formula (I)

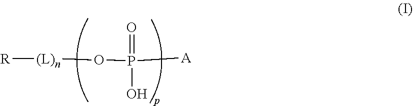

wherein
n is 0 or 1
p is 0 or 1
with the proviso that in case n is 1, p is preferably 1,
and with the proviso that in case n is 0 and p is 0, R is preferably H,
L is a linker, preferably L is a linker comprising or consisting of 2-10 nucleosides, such as 2-5 nucleosides,
R is a GaINAc residue, preferably a trivalent GaINAc residue,
and A is an antisense oligonucleotide residue according to the present invention.

The term “antisense oligonucleotide residue” refers to an antisense oligonucleotide according to the present invention which is attached via its 5′ end to residue R via -(L)ₙ-(O—P(═O)(—OH)—)ₚ—, such as an antisense oligonucleotide as shown in Table 6. Preferred antisense oligonucleotide residues are depicted in FIGS. 1A, 2A, 3A, 4A, 5A, 6A, 7A and 8A.

### The GaINAc Residue R

R is a GaINAc residue, preferably a trivalent GaINAc residue. The term “GaINAc residue” as used herein refers to a residue comprising at least one N-Acetylgalactosamine (GaINAc) moiety, i.e. at least one moiety of formula

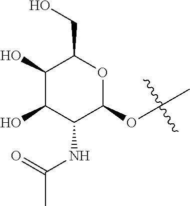 [SMILES: *O[C@@H]1O[C@H](CO)[C@H](O)[C@H](O)[C@H]1NC(C)=O]

The term “trivalent GaINAc residue” as used herein refers to a residue comprising three N-acetylgalactosamine (GaINAc) moieties, i.e. preferably three moieties of formula

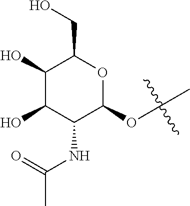 [SMILES: *O[C@@H]1O[C@H](CO)[C@H](O)[C@H](O)[C@H]1NC(C)=O]

Preferably, the GaINAc residue comprises at least one, preferably three GaINAc building blocks (L^{a}) having the following structure,

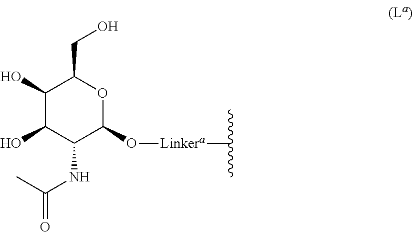

wherein Linker^{a} is selected from alkyl groups, alkyl-oxy-alkyl groups, alkyl groups comprising at least one phosphodiester linkage, alkyl groups comprising at least one amide linkage, alkyl-oxy-alkyl groups comprising at least one phosphodiester linkage and alkyl-oxy-alkyl groups comprising at least one amide linkage.

The term “alkyl” refers to substituted or unsubstituted, linear or branched, alkyl groups, such as C1 to C20 alkyl groups, preferably, C2 to C8, such as C2, C3, C4, C5, C6, C7 or C8, alkyl groups. Preferably the alkyl groups are unsubstituted, more preferably linear and unsubstituted, alkyl groups.

The term “alkyl-oxy-alkyl” groups refers to at least two alkyl groups linked via an oxygen, preferably to ethyl-oxy-ethyl groups, such as —(CH₂—O)ₓ groups with integer x preferably being in the range of from 2 to 20, more preferably in the range of from 2 to 6, such as 2, 3, 4, 5 or 6, more preferably x is 3 or 5.

According to an aspect of the invention, the GaINAc building block (L^{a}) is selected from the group of the following structures (L^{a}).

If more than one residue (L^{a}) is present in a GaINAc residue, such as the three residues in the trivalent GaINAc residue, then all residues are preferably the same.

Most preferably, L^{a} has the structure

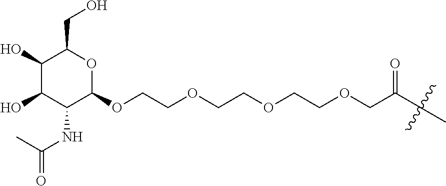 [SMILES: *C(=O)COCCOCCOCCO[C@@H]1O[C@H](CO)[C@H](O)[C@H](O)[C@H]1NC(C)=O]

In case, the conjugate moiety R comprises multiple, such as preferably three, GaINAc moieties, R comprises besides the GaINAc building blocks (L^{a}), a multivalent, preferably a tetravalent, building block (L^{b}), to which the building blocks (L^{a}) are preferably being attached, to the antisense oligonucleotide residue A via -(L)ₙ-(O—P(═O)(—OH)—)ₚ—.

L^{b} is preferably selected from one of the following structures:

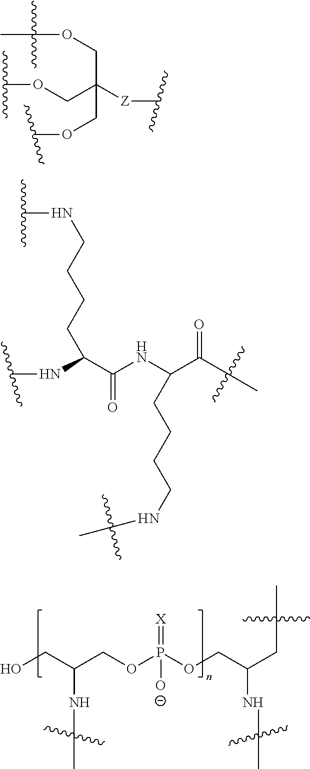

with X being O or S, and with Z being O or NH, and wherein n is of from 1 to 4, preferably 2 or 3, more preferably 2.

More preferably, L^{b} has the structure

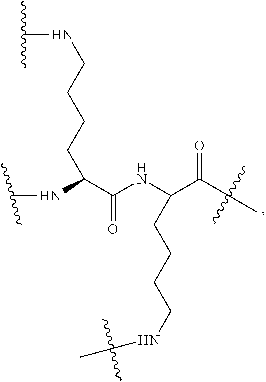 [SMILES: *NCCCCC(NC(=O)[C@H](CCCCN*)N*)C(*)=O]

It is to be understood that, L^{b} has either the structure L^{b}* or the structure L^{b}** or is a mixture thereof. According to a preferred aspect L^{b} is a mixture of L^{b}* and L^{b}**.

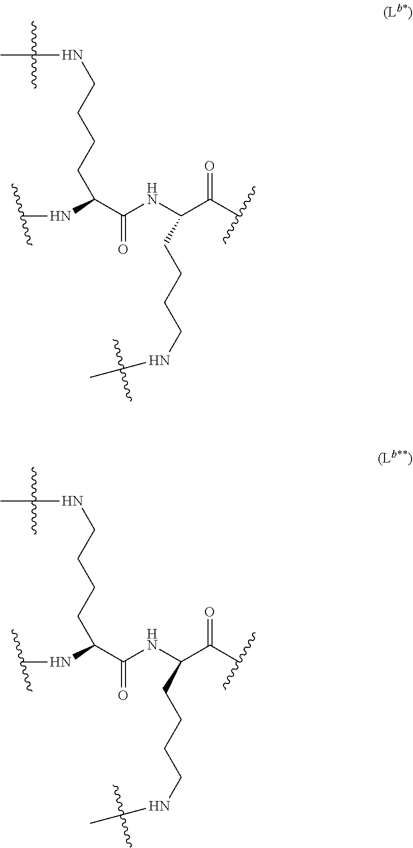 [SMILES: *NCCCC[C@H](NC(=O)[C@H](CCCCN*)N*)C(*)=O ; *NCCCC[C@H](N*)C(=O)N[C@H](CCCCN*)C(*)=O]

Thus, the conjugate moiety R preferably comprises a structure (L^{a})₃-L^{b}-, more preferably R comprises one of the following structures

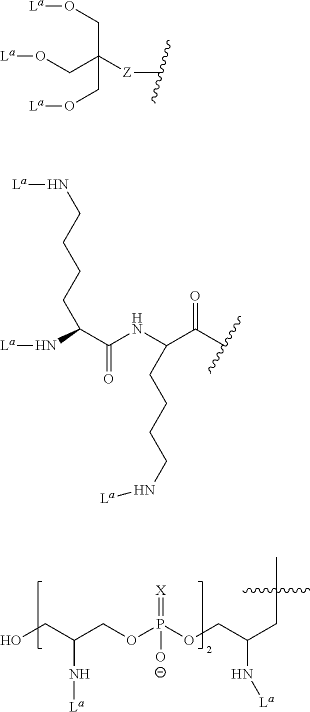

more preferably, the structure

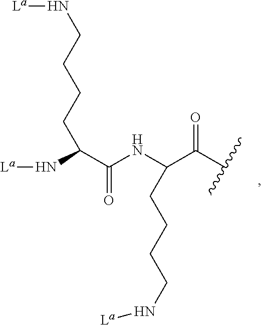

wherein L^{b} is preferably a mixture of L^{b}* and L^{b}**,

and wherein X is O or S, and with Z being O or NH, and wherein n is of from 1 to 3, preferably 2, and with L^{a} being as described above, preferably with L^{a} being selected from the group consisting of

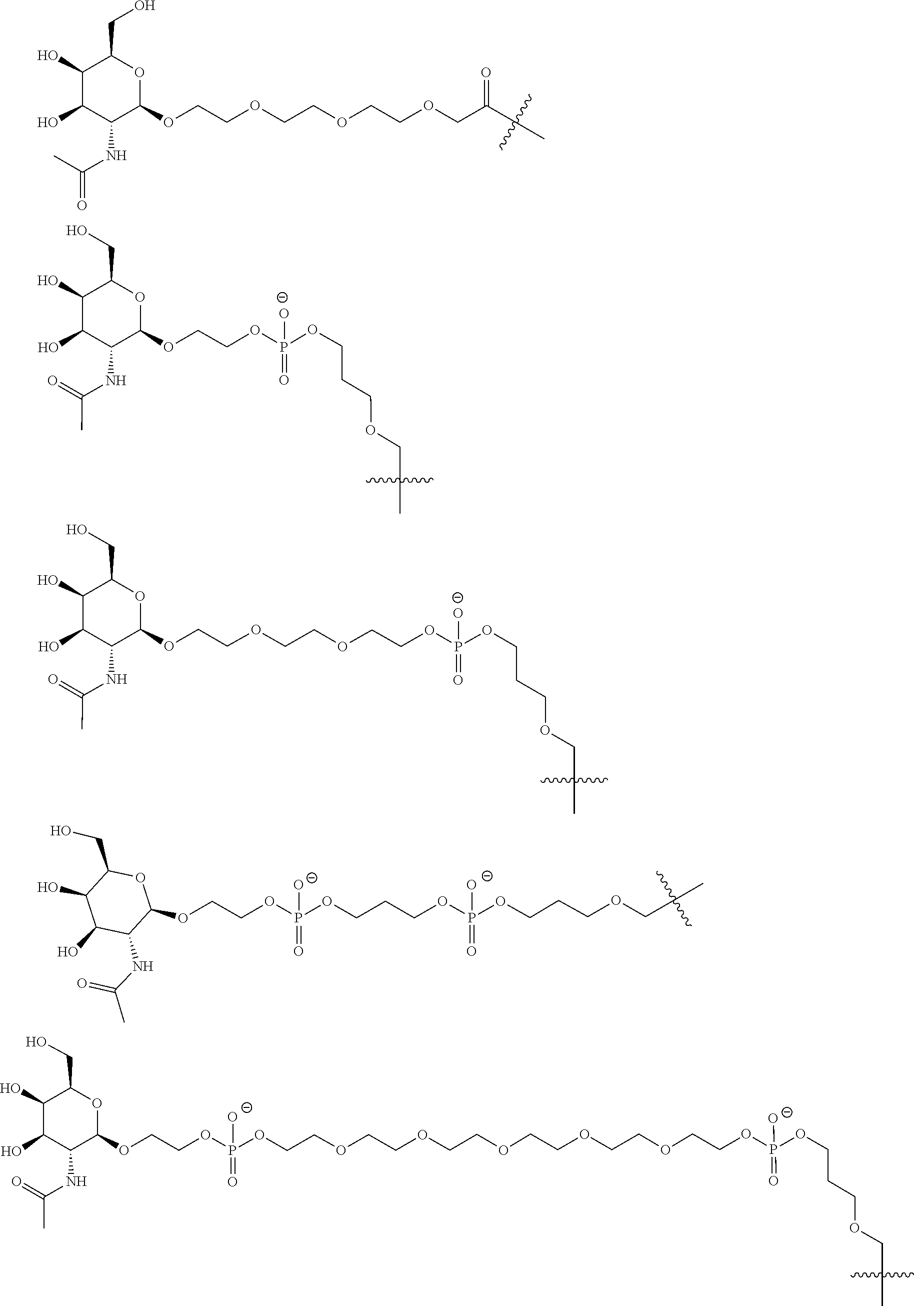 [SMILES: *C(=O)COCCOCCOCCO[C@@H]1O[C@H](CO)[C@H](O)[C@H](O)[C@H]1NC(C)=O ; *COCCCOP(=O)([O-])OCCO[C@@H]1O[C@H](CO)[C@H](O)[C@H](O)[C@H]1NC(C)=O ; *COCCCOP(=O)([O-])OCCOCCOCCO[C@@H]1O[C@H](CO)[C@H](O)[C@H](O)[C@H]1NC(C)=O ; *COCCCOP(=O)([O-])OCCCOP(=O)([O-])OCCO[C@@H]1O[C@H](CO)[C@H](O)[C@H](O)[C@H]1NC(C)=O ; *COCCCOP(=O)([O-])OCCOCCOCCOCCOCCOCCOP(=O)([O-])OCCO[C@@H]1O[C@H](CO)[C@H](O)[C@H](O)[C@H]1NC(C)=O]

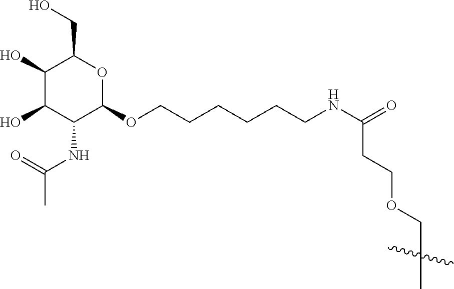 [SMILES: *COCCC(=O)NCCCCCCO[C@@H]1O[C@H](CO)[C@H](O)[C@H](O)[C@H]1NC(C)=O]

and mixtures thereof, wherein preferably all residues (L^{a}) within the GaINAc residue are the same.

In case (L^{a})₃-L^{b} is

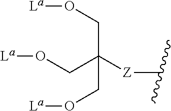

L^{a} is more preferably selected from the group consisting of

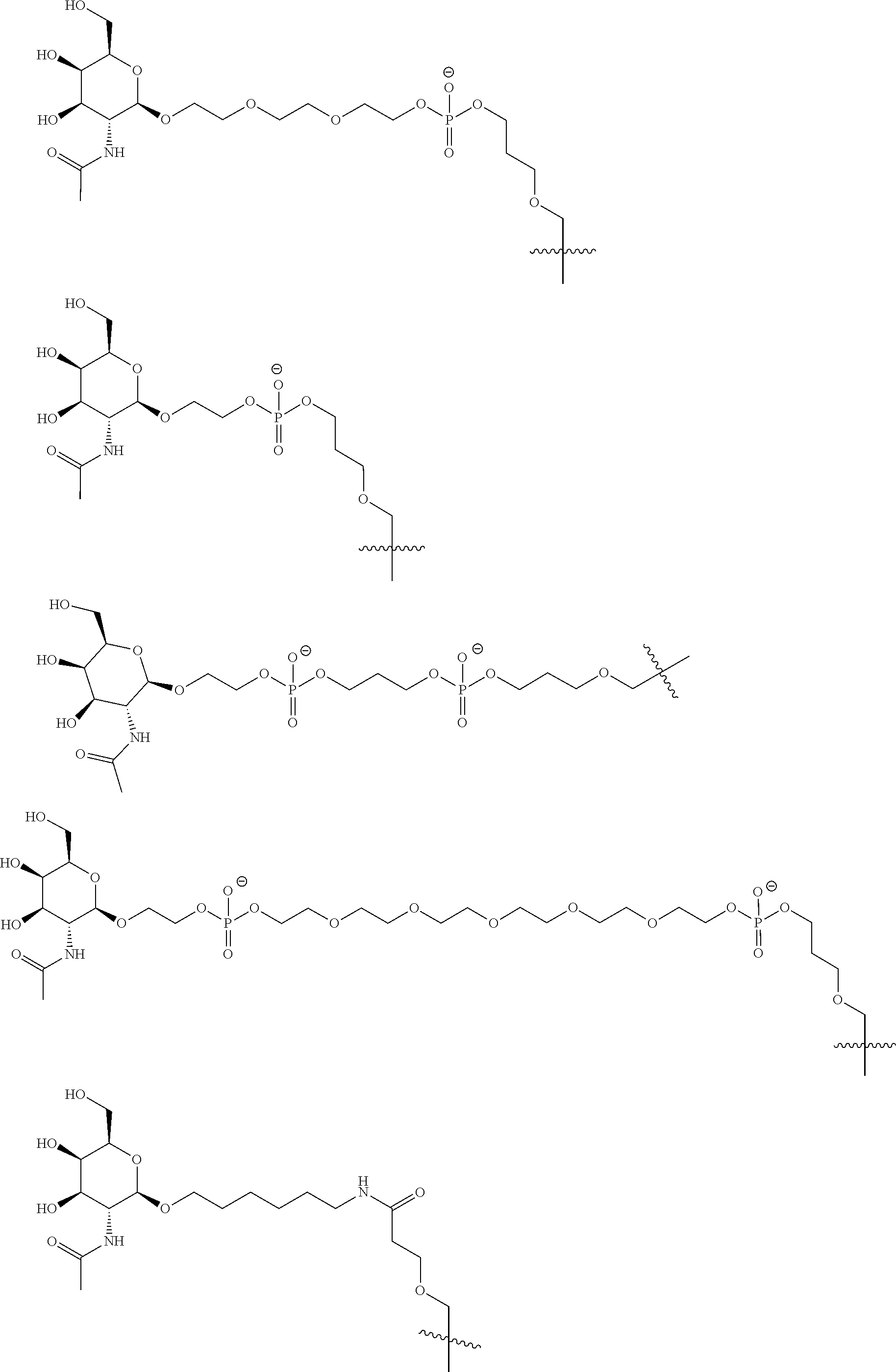 [SMILES: *COCCCOP(=O)([O-])OCCOCCOCCO[C@@H]1O[C@H](CO)[C@H](O)[C@H](O)[C@H]1NC(C)=O ; *COCCCOP(=O)([O-])OCCO[C@@H]1O[C@H](CO)[C@H](O)[C@H](O)[C@H]1NC(C)=O ; *COCCCOP(=O)([O-])OCCCOP(=O)([O-])OCCO[C@@H]1O[C@H](CO)[C@H](O)[C@H](O)[C@H]1NC(C)=O ; *COCCCOP(=O)([O-])OCCOCCOCCOCCOCCOCCOP(=O)([O-])OCCO[C@@H]1O[C@H](CO)[C@H](O)[C@H](O)[C@H]1NC(C)=O ; *COCCC(=O)NCCCCCCO[C@@H]1O[C@H](CO)[C@H](O)[C@H](O)[C@H]1NC(C)=O]

In case (L^{a})₃-L^{b} is

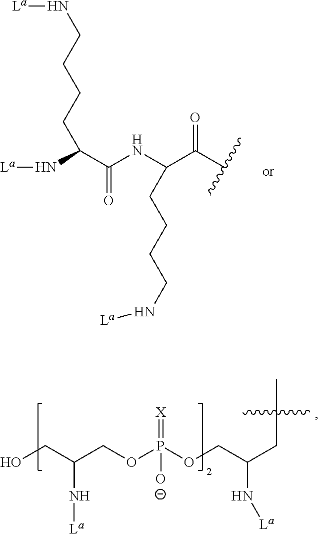

preferably

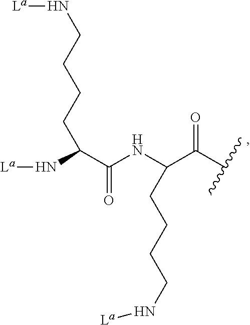

L^{a} is preferably

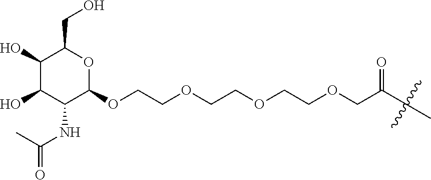 [SMILES: *C(=O)COCCOCCOCCO[C@@H]1O[C@H](CO)[C@H](O)[C@H](O)[C@H]1NC(C)=O]

Optionally, the conjugate moiety R additionally comprises a linker L°. Thus, R preferably has the structure (L^{a})₃-L^{b}-(L^{c})_{c}- with integer c being 1 or 0.

Such linker compounds are known those skilled in the art and are suitably chosen to attach (L^{a})₃-L_{b} to the remaining part of the compound, i.e. to the antisense oligonucleotide residue via -(L)ₙ-(O—P(═O)(—OH)—)ₚ—.

Depending on the structure of L^{b}, L^{c} is selected from the group consisting of alkyl, alkyl-oxy-alkyl, amino-alkyl (—NH-alkyl-), amino-alkyl-oxy-alkyl, unnatural amino acid residues, and natural amino acid residues. According to one aspect of the invention, L^{c} is a substituted or unsubstituted lysine group.

According to one aspect of the invention, R is (L^{a})₃-L^{b}-(L^{c})_{c} with c=1 and (L^{a})₃-L_{b} is

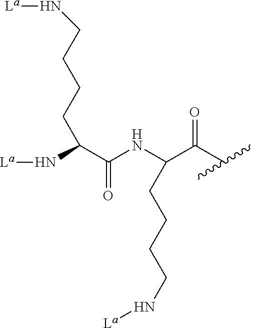

L^{c} is preferably an amino-alkyl group or an amino acid, such as a substituted or unsubstituted lysine group, in particular L^{c} is e.g. selected from the group consisting of

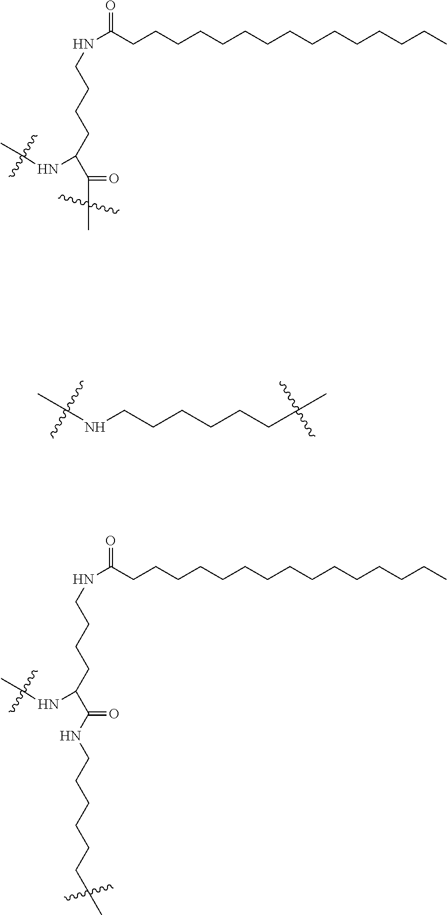 [SMILES: *NC(CCCCNC(=O)CCCCCCCCCCCCCCC)C(*)=O ; *CCCCCCN* ; *CCCCCCNC(=O)C(CCCCNC(=O)CCCCCCCCCCCCCCC)N*]

with the amino group being attached to the carbonyl group of L^{b} thereby forming an amide bond. Preferred residues R according to this aspect are depicted in FIG. 5A1, 5A2; 5C1, 5C2, 5D1, 5D2. Thus, according to one aspect of the invention, R is selected from the group consisting of the residues depicted in FIGS. 5A1, 5A2; 5C1, 5C2, 5D1 and 5D2.

According to a further aspect of the invention, R has the structure (L^{a})₃-L^{b}-(L^{c})_{c} with c being 0, and wherein (L^{a})₃-L_{b} is

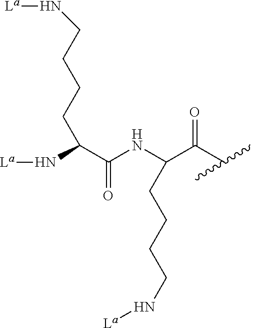

Preferred residues R according to this aspect of the invention are depicted in FIGS. 5B1 and 5B2.

According to a further aspect of the invention, R has the structure (L^{a})₃-L^{b}-(L^{c})_{c} with (L^{a})₃-L_{b} being

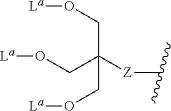

and with Z being O. In this case, c is preferably 1 and L^{c} is preferably an alkyl group, more preferably a C3 to C6 alkyl group, more preferably a propyl group, most preferably a n-propyl group. Preferred residues R according to this aspect are depicted in FIGS. 5E1, 5F1, 5G1 and 5H1. Thus, according to one aspect of the invention, R is selected from the group consisting of the residues depicted in FIGS. 5E1, 5F1, 5G1 and 5H1 According to a further aspect of the invention, R has the structure (L^{a})₃-L^{b}-(L^{c})_{c} with (L^{a})₃-L_{b} being

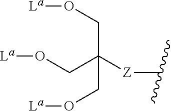

and with Z being NH. In this case c is preferably 1 and L^{c} is preferably an alkyl group, an amino acid comprising group or a group having the following structure:

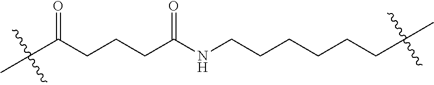 [SMILES: *CCCCCCNC(=O)CCCC(*)=O]

In particular, in this case L^{c} is

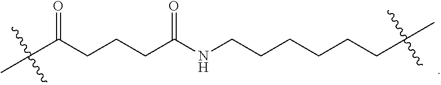 [SMILES: *CCCCCCNC(=O)CCCC(*)=O]

A preferred residue R according to this aspect of the invention is depicted in FIG. 5J1.

According to a further aspect of the invention, R has the structure (L^{a})₃-L^{b}-(L^{c})_{c} with (L^{a})₃-L_{b} being

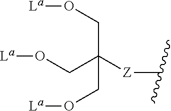

and with Z being NH and c being 0. A preferred residue R according to this aspect of the invention is depicted in FIG. 5I1.

According to one aspect of the invention, R is (L^{a})₃-L^{b}-(L^{c})_{c} with c=0 and wherein (L^{a})₃-L_{b} is

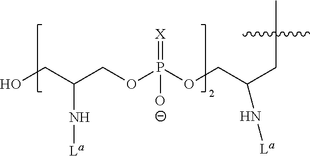

Preferred residues R according to this aspect of the invention are depicted in FIGS. 5L1 and 5L2.

Thus, R is preferably selected from the residues depicted in FIGS. 5A1, 5A2; 5B1, 5B2, 5C1, 5C2, 5D1, 5D2, 5E1, 5F1, 5G1, 5H1, 5I1, 5J1, 5L1 5L2, and mixtures thereof, such as stereoisomeric mixtures of 5A1 and 5A2; of 511 and 5B2, of 5C1 and 5C2 or 5D1 and D2, more preferably R is selected from the residues depicted in 5D1, 5D2 and a mixture thereof, more preferably R is a mixture of the residues depicted in 5D1 and 5D2, such as a mixture having a molar ratio of 5D1 to 5D2 in the range of from 10:90 to 90:10, such as in the range of from 30:70 to 70:30, such as in the range of from 45:55 to 55:45.

Thus, compound (I) is preferably selected from the compounds depicted in FIGS. 6A1, 6A2; 6B1, 6B2, 6C1, 6C2, 6D1, 6D2, 6E1, 6F1, 6G1, 6H1, 6I1, 6J1, 6L1, 6L2 and mixtures thereof, such as stereoisomeric mixtures of 6A1 and 6A2; of 6B1 and 6B2, of 6C1 and 6C2 or of 6D1 and 6D2, more preferably compound (I) is selected from the group consisting of the compounds depicted in 6D1, 6D2, and mixtures thereof, more preferably compound (I) is a mixture of the compounds depicted in 6D1 and 6D2, such as a mixture having a molar ratio of 6D1 to 6D2 in the range of from 10:90 to 90:10, such as in the range of from 30:70 to 70:30, such as in the range of from 45:55 to 55:45.

### The Linker L

In the above formula, L is a linker as defined herein, preferably L is a linker comprising or consisting of 2-10 nucleosides, such as 2-5 nucleosides, such as 2 nucleosides, wherein optionally the nucleosides are phosphodiester linked nucleosides.

As the linker L comprises between 1 and 10 linked nucleosides, such as 1, 2, 3, 4, 5, 6, 7, 8, 9 or 10 linked nucleosides, such as between 2 and 6 linked nucleosides, such as between 2 and 5 linked nucleosides, such as between 2 and 4 linked nucleosides. In some embodiments, the linker comprises two linked nucleotides. Thus, the nucleosides may be DNA nucleosides. Typically, the nucleosides are linked via phosphodiester internucleoside linkages. Moreover, the linker L may be linked to the antisense compound via a phosphodiester internucleoside linkage. Further, the linker L is linked to conjugate moiety R via a suitable function group, such as e.g., via an amide, an amine, an ether, an ester, a phosphodiester (—O—P(═O)(—OH)—O—) or thiophosphodiester (—O—P(═S)(—OH)—O—) linkage. It is to be understood that L may optionally additionally comprise alkyl groups or alkyl-oxy-alkyl groups between the nucleosides and the functional group linking L to R. In this case, the nucleosides are preferably linked via a phosphodiester bond to the alkyl groups or alkyl-oxy-alkyl group which in turn is linked to R via a suitable function group, such as e.g., via an amide, an amine, an ether, an ester, a phosphodiester (—O—P(═O)(—OH)—O—) or thiophosphodiester (—O—P(═S)(—OH)—O—) bond

According to a preferred embodiment L is

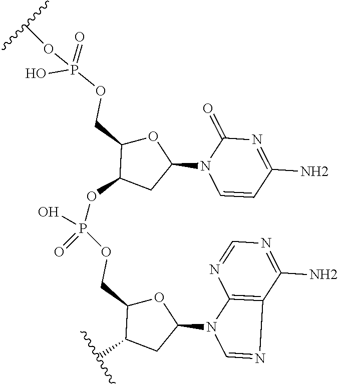 [SMILES: *OP(=O)(O)OC[C@H]1O[C@@H](n2ccc(N)nc2=O)C[C@H]1OP(=O)(O)OC[C@H]1O[C@@H](n2cnc3c(N)ncnc32)C[C@@H]1*]

### The Antisense (A) Oligonucleotide Residue

A is an antisense oligonucleotide residue according to the present invention, such an antisense oligonucleotide shown in Table 6, being attached via its 5′ prime end to R via -(L)ₙ-(O—P(═O)(—OH)—)ₚ. Preferably, A is an antisense oligonucleotide residue selected from the residues depicted in FIGS. 1A, 2A 3A and 4.

Thus, compound (I) is preferably selected from the compounds depicted in FIGS. 6A1, 6A2; 6B1, 6B2, 6C1, 6C2, 6D1, 6D2, 6E1, 6F1, 6G1, 6H1, 611, 6J1, 6L1 6L2, and mixtures thereof, such as stereoisomeric mixtures of 6A1 and 6A2; of 6B1 and 6B2, of 6C1 and 6C2 or of 6D1 and 6D2, more preferably compound (I) is selected from the compounds depicted in 6D1 and 6D2, and a mixture thereof, more preferably compound (I) is a mixture of compound 6D1 and 6D2, preferably with A being selected from the antisense oligonucleotide shown in Table 6, preferably with A being an antisense oligonucleotide residue selected from the residues depicted in FIGS. 1A, 2A 3A, 4A, 5A, 6A, 7A and 8A. and with L is a linker comprising or consisting of 2-10 nucleosides, such as 2-5 nucleosides, such as 2 nucleosides, wherein optionally the nucleosides are phosphodiester linked nucleosides,
more preferably wherein L is

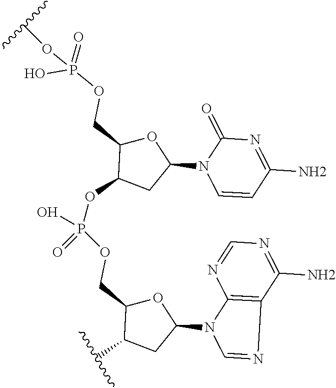 [SMILES: *OP(=O)(O)OC[C@H]1O[C@@H](n2ccc(N)nc2=O)C[C@H]1OP(=O)(O)OC[C@H]1O[C@@H](n2cnc3c(N)ncnc32)C[C@@H]1*]

In a further aspect, R is a residue having the structure (I)

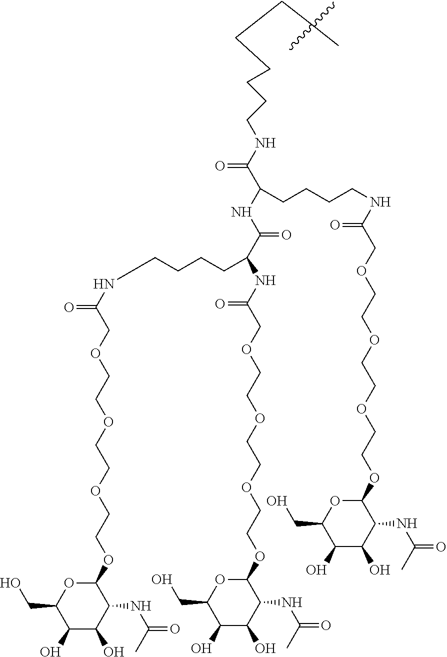 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

L is a linker as defined herein, preferably L is a linker comprising or consisting of 2-10 nucleosides, such as 2-5 nucleosides, such as 2 nucleosides, wherein optionally the nucleosides are phosphodiester linked nucleosides, more preferably L is

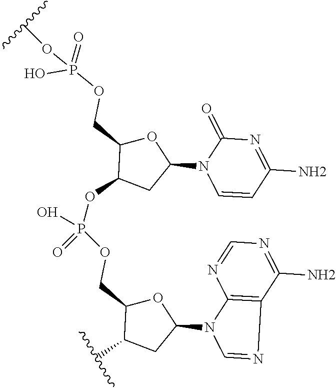 [SMILES: *OP(=O)(O)OC[C@H]1O[C@@H](n2ccc(N)nc2=O)C[C@H]1OP(=O)(O)OC[C@H]1O[C@@H](n2cnc3c(N)ncnc32)C[C@@H]1*]

and

A is an antisense oligonucleotide according to the present invention, such an as antisense oligonucleotide shown in Table 6.

According to one aspect of the invention, A is an antisense oligonucleotide residue selected from the residues depicted in FIGS. 1A, 2A 3A and 4A,
for example A is an antisense oligonucleotide of the following formula:

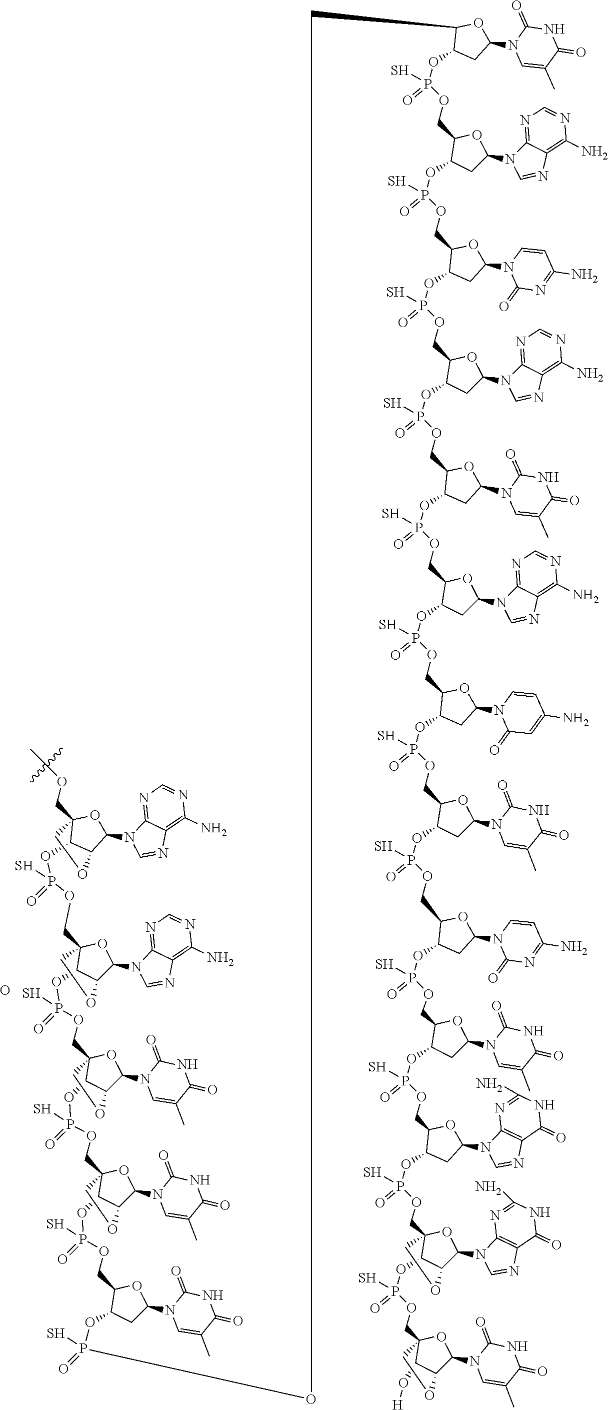

The invention provides pharmaceutical compositions comprising the antisense oligonucleotide of the invention or the conjugate thereof, and a pharmaceutically acceptable diluents, carriers, salts and/or adjuvants.

The invention provides for a pharmaceutically acceptable salt of the antisense oligonucleotide of the invention or the conjugate thereof. In some embodiments, the pharmaceutically acceptable salt is selected from the group consisting of a sodium salt, a potassium salt and an ammonium salt.

The invention provides for a pharmaceutical solution of the antisense oligonucleotide of the invention or the conjugate thereof, wherein the pharmaceutical solution comprises the antisense oligonucleotide of the invention or the conjugate thereof and a pharmaceutically acceptable solvent, such as saline.

The invention provides for the antisense oligonucleotide of the invention or the conjugate thereof in solid powdered form, such as in the form of a lyophilized powder.

The invention provides for a pharmaceutically acceptable salt of the antisense oligonucleotide of the invention or the conjugate thereof.

The invention provides for a pharmaceutically acceptable salt of the antisense oligonucleotide according to the invention, or the conjugate of the invention, wherein the pharmaceutically acceptable salt is a sodium or potassium salt.

The invention provides for a pharmaceutical composition comprising the antisense oligonucleotide of the invention, or the conjugate of the invention, or the salt of the invention and a pharmaceutically acceptable diluent, solvent, carrier, salt and/or adjuvant.

The invention provides for a method for inhibiting RTEL1 expression in a target cell, which is expressing RTEL1, said method comprising administering an antisense oligonucleotide of the invention, or the conjugate of the invention, or the salt of the invention, or the composition of the invention in an effective amount to said cell. The method may be an in vivo method or an in vitro method.

The invention provides for a method for treating and/or preventing an HBV infection in a subject such as a human, comprising administering a therapeutically or prophylactically effective amount of an antisense oligonucleotide of the invention, or the conjugate of the invention, or the salt of the invention, or the composition of the invention, such as to treat and/or prevent an HBV infection, such as a chronic HBV infection.

In some embodiments, the antisense oligonucleotide of the invention, or the conjugate of the invention, or the salt of the invention, or the pharmaceutical composition of the invention, is for the use in the treatment and/or prevention of an HBV infection, such as a chronic HBV infection.

The invention provides the antisense oligonucleotide of the invention, or the conjugate of the invention, or the pharmaceutical composition, or the salt of the invention for use in medicine.

In a further aspect, the invention provides methods for inhibition of RTEL1 expression in a target cell, which is expressing RTEL1, by administering an antisense oligonucleotide of the invention, or conjugate of the invention in an effective amount to said cell. In a further aspect, the invention provides methods for in vivo or in vitro method for inhibition of RTEL1 expression in a target cell, which is expressing RTEL1, by administering an antisense oligonucleotide, or the conjugate of the invention in an effective amount to said cell. The cell may for example be a human cell, such as a liver cell, such as a hepatocyte.

In a further aspect, the invention provides methods for reducing cccDNA in an HBV infected cell, by administering an antisense oligonucleotide of the invention, or conjugate of the invention in an effective amount to said cell. In a further aspect, the invention provides methods for in vivo or in vitro method for reducing cccDNA in an HBV infected cell, by administering an antisense oligonucleotide of the invention, or the conjugate of the invention in an effective amount to said cell.

In a further aspect, the invention provides methods for treating and/or preventing an HBV infection, such as a chronic HBV infection.

In a further aspect the antisense oligonucleotide of the invention, or the conjugate of the invention, or pharmaceutical composition of the invention is used in the treatment and/or prevention of viral liver infections such as HBV, HCV, HDV or a parasite infections such as malaria, toxoplasmosis, leishmaniasis and trypanosomiasis or liver cancer or metastases in the liver.

In a further aspect, the invention provides the antisense oligonucleotide, or the conjugate of the invention, or the pharmaceutical composition of the invention, for use in the manufacture of a medicament for the treatment and/or prevention of an HBV infection, such as a chronic HBV infection.

In a further aspect, the invention provides the antisense oligonucleotide, or the conjugate of the invention, or the pharmaceutical composition of the invention, for use in the manufacture of an antiviral drug.

The invention provides for the antisense oligonucleotide of the invention, or the conjugate of the invention, or the pharmaceutical composition of the invention, for use in the treatment of an HBV infection, such as a chronic HBV infection.

### SEQUENCE LISTING

The sequence listing submitted with this application is hereby incorporated by reference. In the event of a discrepancy between the sequence listing and the specification or figures, the information disclosed in the specification (including the figures) shall be deemed to be correct.

### BRIEF DESCRIPTION OF FIGURES

FIG. 1: Compound 3_1 (SEQ ID NO: 3) conjugated to a trivalent GaINAc moiety via a phosphodiester linked DNA dinucleotide

FIG. 1A: Residue A of Compound 3_1 (SEQ ID NO: 3)

FIG. 2: Compound 4_1 (SEQ ID NO: 4) conjugated to a trivalent GaINAc moiety via a phosphodiester linked DNA dinucleotide

FIG. 2A: Residue A of Compound 4_1 (SEQ ID NO: 4)

FIG. 3: Compound 6_1 (SEQ ID NO: 6) conjugated to a trivalent GaINAc moiety via a phosphodiester linked DNA dinucleotide

FIG. 3A: Residue A of Compound 6_1 (SEQ ID NO: 6)

FIG. 4: Compound 5_1 (SEQ ID NO: 5) conjugated to a trivalent GaINAc moiety via a phosphodiester linked DNA dinucleotide

FIG. 4A: Residue A of Compound 5_1 (SEQ ID NO: 5)

FIG. 5A1—

FIG. 5L2: FIG. 5A1 through FIG. 5L2 illustrate exemplary GaINAc moieties. The compound in FIG. 5L is composed of monomeric GaINAc phosphoramidites added to the oligonucleotide while still on the solid support as part of the synthesis, X is S or O, Y is S or O, and n=1-3 (see WO 2017/178656). FIG. 5B and FIG. 5D are also termed GaINAc2 or GN2 herein, without and with C6 linker, respectively.

FIG. 6A1—

FIG. 6L2: FIG. 6A1 through FIG. 6L2 illustrate exemplary antisense oligonucleotide conjugates, wherein the oligonucleotide is represented by the term “A” as described above. Compounds in FIG. 6A-D comprise a di-lysine brancher molecule, a PEG3 spacer and three terminal GaINAc carbohydrate moieties. In the compounds in FIG. 6A (FIG. 6A-1 and FIG. 6A-2 show two different diastereoisomers of the same compound) and FIG. 6B (FIG. 6B-1 and FIG. 6B-2 show two different diastereoisomers of the same compound) the oligonucleotide is attached directly to the asialoglycoprotein receptor targeting conjugate moiety without an alkyl linker. In the compounds in FIG. 6C (FIG. 6C-1 and FIG. 6C-2 show two different diastereoisomers of the same compound) and FIG. 6D (FIG. 6D-1 and FIG. 6D-2 show two different diastereoisomers of the same compound) the oligonucleotide is attached to the asialoglycoprotein receptor targeting conjugate moiety via a C6 linker. The compounds in FIG. 6E-K comprise a commercially available trebler brancher molecule and spacers of varying length and structure and three terminal GaINAc carbohydrate moieties. The compound in FIG. 6L is composed of monomeric GaINAc phosphoramidites added to the oligonucleotide while still on the solid support as part of the synthesis, wherein X=S or O, and independently Y=S or O, and n=1-3 (see WO 2017/178656).

FIG. 7: Testing oligonucleotide CMP ID Nos 3_1, 4_1, 5_1 and 6_1 in vitro for concentration dependent potency and efficacy in human cell line MDA-MB-231.

FIG. 8: Testing oligonucleotide CMP ID Nos 3_1, 4_1, 5_1 and 6_1 and prior art compounds (CMP ID Nos 7_1 to 23_1) in vitro for concentration dependent potency and efficacy in human cell line MDA-MB-231.

FIG. 9A—

FIG. 9E: FIG. 9A through FIG. 9E illustrate testing oligonucleotide antisense molecules and shorter metabolites thereof in vitro for concentration dependent potency and efficacy in human cell line MDA-MB-231. FIG. 9A) CMP ID 5_1, FIG. 9B) CMP ID 7_1, FIG. 9C) CMP ID 8_1, FIG. 9D) CMP ID 9_1, FIG. 9E) CMP ID 10_1.

FIG. 10: Study protocol for in vivo analysis in mice treated with GaINAc-conjugated CMP ID 5_1

FIG. 11: Level of RTEL1 mRNA in liver tissue from PXB-mice treated with GaINAc-conjugated oligonucleotide antisense CMP ID Nos 3_1 and 5_1

FIG. 12: Study protocol for in vivo analysis of HBV-infected PXB-mice treated with GaINAc-conjugated CMP ID 5_1.

FIG. 13A—

FIG. 13B: FIG. 13A and FIG. 13B illustrate RTEL1 mRNA level and HBV cccDNA level in liver tissue from HBV-infected PXB-mice treated with GaINAc-conjugated CMP ID 5_1 and a control: FIG. 13A) Day 105, FIG. 13B) Day 56.

### DEFINITIONS

### HBV Infection

The term “hepatitis B virus infection” or “HBV infection” is commonly known in the art and refers to an infectious disease that is caused by the hepatitis B virus (HBV) and affects the liver. A HBV infection can be an acute or a chronic infection. Chronic hepatitis B virus (CHB) infection is a global disease burden affecting 248 million individuals worldwide. Approximately 686,000 deaths annually are attributed to HBV-related end-stage liver diseases and hepatocellular carcinoma (HCC) (GBD 2013; Schweitzer et al., 2015). WHO projected that without expanded intervention, the number of people living with CHB infection will remain at the current high levels for the next 40-50 years, with a cumulative 20 million deaths occurring between 2015 and 2030 (WHO 2016). CHB infection is not a homogenous disease with singular clinical presentation. Infected individuals have progressed through several phases of CHB-associated liver disease in their life; these phases of disease are also the basis for treatment with standard of care (SOC). Current guidelines recommend treating only selected CHB-infected individuals based on three criteria—serum ALT level, HBV DNA level, and severity of liver disease (EASL, 2017). This recommendation was due to the fact that SOC i.e. nucleos(t)ide analogs (NAs) and pegylated interferon-alpha (PEG-IFN), are not curative and must be administered for long periods of time thereby increasing their safety risks. NAs effectively suppress HBV DNA replication; however, they have very limited/no effect on other viral markers. Two hallmarks of HBV infection, hepatitis B surface antigen (HBsAg) and covalently closed circular DNA (cccDNA) are the main targets of novel drugs aiming for HBV cure. In the plasma of CHB individuals, HBsAg subviral (empty) particles outnumber HBV virions by a factor of 10³ to 10⁵ (Ganem & Prince, 2014); its excess is believed to contribute to immunopathogenesis of the disease, including inability of individuals to develop neutralizing anti-HBs antibody, the serological marker observed following resolution of acute HBV infection.

cccDNA (Covalently Closed Circular DNA)

cccDNA is the viral genetic template that resides in the nucleus of infected hepatocytes, where it gives rise to all HBV RNA transcripts needed for productive infection and is responsible for viral persistence during natural course of chronic HBV infection (Locarnini & Zoulim, 2010 Antivir Ther. 15 Suppl 3:3-14. doi: 10.3851/IMP1619). Acting as a viral reservoir, cccDNA is the source of viral rebound after cessation of treatment, necessitating long term, often, lifetime treatment. PEG-IFN can only be administered to a small subset of CHB due to its various side effects.

Consequently, novel therapies that can deliver a complete cure, defined by degradation or elimination of HBV cccDNA, to the majority of CHB patients are highly needed.

### Compound

Herein, the term “compound” means any molecule capable of inhibition RTEL1 expression or activity. Particular compounds of the invention are antisense oligonucleotides according to the invention or any conjugate comprising such a nucleic acid molecule. For example, herein the compound may be a nucleic acid molecule targeting RTEL1, in particular an antisense oligonucleotide.

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides such as 2′ sugar modified nucleosides. The oligonucleotide of the invention may comprise one or more modified internucleoside linkages, such as one or more phosphorothioate internucleoside linkages.

### Antisense Oligonucleotides

The term “antisense oligonucleotide” as used herein is defined as oligonucleotides capable of modulating expression of a target gene by hybridizing to a target nucleic acid, in particular, to a contiguous sequence on a target nucleic acid. Antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide.

In some embodiments, the single stranded antisense oligonucleotide of the invention may not contain RNA nucleosides.

Advantageously, the antisense oligonucleotide of the invention comprises one or more modified nucleosides or nucleotides, such as 2′ sugar modified nucleosides. Furthermore, it is advantageous that the nucleosides which are not modified are DNA nucleosides.

### Contiguous Nucleotide Sequence

The term “contiguous nucleotide sequence” refers to the region of the oligonucleotide, which is complementary to the target nucleic acid. The term is used interchangeably herein with the term “contiguous nucleobase sequence” and the term “oligonucleotide motif sequence”. In some embodiments, all the nucleosides of the oligonucleotide constitute the contiguous nucleotide sequence. In some embodiments, the oligonucleotide comprises the contiguous nucleotide sequence, such as a F-G-F′ gapmer region, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid. In some embodiments, the nucleobase sequence of the antisense oligonucleotide is the contiguous nucleotide sequence.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. Advantageously, one or more of the modified nucleosides of the antisense oligonucleotide of the invention comprise a modified sugar moiety. The term “modified nucleoside” may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise one or more modified internucleoside linkages such as a one or more phosphorothioate internucleoside linkages, or one or more phosphorodithioate internucleoside linkages.

In some embodiments, at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate.

In some advantageous embodiments, all the internucleoside linkages of the contiguous nucleotide sequence of the oligonucleotide are phosphorothioate, or all the internucleoside linkages of the oligonucleotide are phosphorothioate linkages.

It is recognized that, as disclosed in EP 2 742 135, antisense oligonucleotides may comprise other internucleoside linkages (other than phosphodiester, phosphorothioate and phosphorodithioate), for example alkyl phosphonate/methyl phosphonate internucleoside, which according to EP 2 742 135 may for example be tolerated in an otherwise DNA phosphorothioate the gap region.

### Nucleobase

The term nucleobase includes the purine (e.g. adenine and guanine) and pyrimidine (e.g. uracil, thymine and cytosine) moiety present in nucleosides and nucleotides, which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments, the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiazolo-cytosine, 5-propynyl-cytosine, 5-propynyl-uracil, 5-bromouracil 5-thiazolo-uracil, 2-thio-uracil, 2′ thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides comprising sugar modified nucleosides and DNA nucleosides. The antisense oligonucleotide of the invention is advantageously a chimeric oligonucleotide.

### Complementarity

The term “complementarity” describes the capacity for Watson-Crick base-pairing of nucleosides/nucleotides. Watson-Crick base pairs are guanine (G)—cytosine (C) and adenine (A)—thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1 M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments, the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments, the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### The Target

The term “target” as used herein refers to the mammalian protein RTEL1 (“Regulator of telomere elongation helicase 1), alternatively known as “KIAA1088” or “C20ORF41” or “Regulator of telomere length” or “Telomere length regulator” or “Chromosome 20 open reading frame 41”. The Homo sapiens RTEL1 gene is located at chromosome 20, 63,657,810 to 63,696,253, complement (Homo sapiens Updated Annotation, Release 109.20200228, GRCh38.p 13). The RTEL1 protein is an ATP-dependent DNA helicase implicated in telomere-length regulation, DNA repair and the maintenance of genomic stability. The amino acid sequence of human RTEL1 is known in the art and can be assessed via UniProt, see UniProt entry Q9NZ71 for human RTEL1, hereby incorporated by reference.

### Target Nucleic Acid

According to the present invention, the target nucleic acid is a nucleic acid, which encodes mammalian RTEL1 and may for example be a gene, a RNA, a mRNA, and pre-mRNA, a mature mRNA or a cDNA sequence. The target may therefore be referred to as an RTEL1 target nucleic acid.

The antisense oligonucleotide of the invention may for example target exon regions of a mammalian RTEL1, or may for example target intron region in the RTEL1 pre-mRNA. The human RTEL1 gene encodes 15 transcripts of these 7 are protein coding and therefore potential nucleic acid targets. Table 3 lists predicted exon and intron regions of the 7 transcripts, as positioned on the human RTEL1 pre-mRNA of SEQ ID NO: 1.

TABLE 3
Transcript-, exonic- and intronic regions in the human RTEL1 pre-mRNA
(SEQ ID NO: 1) for the different protein coding RTEL1 mRNA transcripts

| | Transcript region | | Exonic regions | | | Intron regions | | |
|---|---|---|---|---|---|---|---|---|
| Transcript ID | start | end | Exon | start | end | intron | start | end |
| RTEL1-205 | 1 | 38444 | 1 | 1 | 657 | 1 | 657 | 1424 |
| ENST00000370018 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4737 |
| | | | 5 | 4737 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | 10 | 16284 | 20336 |
| | | | 11 | 20336 | 20374 | 11 | 20374 | 20459 |
| | | | 12 | 20459 | 20537 | 12 | 20537 | 22040 |
| | | | 13 | 22040 | 22137 | 13 | 22137 | 22855 |
| | | | 14 | 22855 | 22910 | 14 | 22910 | 27714 |
| | | | 15 | 27714 | 27788 | 15 | 27788 | 27982 |
| | | | 16 | 27982 | 28063 | 16 | 28063 | 29829 |
| | | | 17 | 29829 | 29961 | 17 | 29961 | 30128 |
| | | | 18 | 30128 | 30241 | 18 | 30241 | 30330 |
| | | | 19 | 30330 | 30370 | 19 | 30370 | 30492 |
| | | | 20 | 30492 | 30577 | 20 | 30577 | 30719 |
| | | | 21 | 30719 | 30796 | 21 | 30796 | 31246 |
| | | | 22 | 31246 | 31323 | 22 | 31323 | 31693 |
| | | | 23 | 31693 | 31839 | 23 | 31839 | 31941 |
| | | | 24 | 31941 | 32056 | 24 | 32056 | 32278 |
| | | | 25 | 32278 | 32401 | 25 | 32401 | 32485 |
| | | | 26 | 32485 | 32632 | 26 | 32632 | 32996 |
| | | | 27 | 32996 | 33138 | 27 | 33138 | 33933 |
| | | | 28 | 33933 | 34028 | 28 | 34028 | 34996 |
| | | | 29 | 34996 | 35194 | 29 | 35194 | 35334 |
| | | | 30 | 35334 | 35474 | 30 | 35474 | 36563 |
| | | | 31 | 36563 | 36679 | 31 | 36679 | 36932 |
| | | | 32 | 36932 | 37165 | 32 | 37165 | 37257 |
| | | | 33 | 37257 | 37412 | 33 | 37412 | 37519 |
| | | | 34 | 37519 | 37671 | 34 | 37671 | 37969 |
| | | | 35 | 37969 | 38444 | | | |
| RTEL1-203 | 485 | 38433 | 1 | 485 | 657 | 1 | 657 | 1424 |
| ENST00000360203 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4737 |
| | | | 5 | 4737 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | 10 | 16284 | 20336 |
| | | | 11 | 20336 | 20374 | 11 | 20374 | 20459 |
| | | | 12 | 20459 | 20537 | 12 | 20537 | 22040 |
| | | | 13 | 22040 | 22137 | 13 | 22137 | 22855 |
| | | | 14 | 22855 | 22910 | 14 | 22910 | 27714 |
| | | | 15 | 27714 | 27788 | 15 | 27788 | 27982 |
| | | | 16 | 27982 | 28063 | 16 | 28063 | 29829 |
| | | | 17 | 29829 | 29961 | 17 | 29961 | 30128 |
| | | | 18 | 30128 | 30241 | 18 | 30241 | 30330 |
| | | | 19 | 30330 | 30370 | 19 | 30370 | 30492 |
| | | | 20 | 30492 | 30577 | 20 | 30577 | 30719 |
| | | | 21 | 30719 | 30796 | 21 | 30796 | 31246 |
| | | | 22 | 31246 | 31323 | 22 | 31323 | 31693 |
| | | | 23 | 31693 | 31839 | 23 | 31839 | 31941 |
| | | | 24 | 31941 | 32056 | 24 | 32056 | 32278 |
| | | | 25 | 32278 | 32401 | 25 | 32401 | 32485 |
| | | | 26 | 32485 | 32632 | 26 | 32632 | 32996 |
| | | | 27 | 32996 | 33138 | 27 | 33138 | 33933 |
| | | | 28 | 33933 | 34028 | 28 | 34028 | 34996 |
| | | | 29 | 34996 | 35194 | 29 | 35194 | 35334 |
| | | | 30 | 35334 | 35474 | 30 | 35474 | 36563 |
| | | | 31 | 36563 | 36679 | 31 | 36679 | 36932 |
| | | | 32 | 36932 | 37165 | 32 | 37165 | 37257 |
| | | | 33 | 37257 | 37412 | 33 | 37412 | 37519 |
| | | | 34 | 37519 | 37841 | 34 | 37841 | 37969 |
| | | | 35 | 37969 | 38433 | | | |
| RTEL1-212 | 482 | 38171 | 1 | 482 | 657 | 1 | 657 | 1424 |
| ENST00000508582 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4665 |
| | | | 5 | 4665 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | 10 | 16284 | 20336 |
| | | | 11 | 20336 | 20374 | 11 | 20374 | 20459 |
| | | | 12 | 20459 | 20537 | 12 | 20537 | 22040 |
| | | | 13 | 22040 | 22137 | 13 | 22137 | 22855 |
| | | | 14 | 22855 | 22910 | 14 | 22910 | 27714 |
| | | | 15 | 27714 | 27788 | 15 | 27788 | 27982 |
| | | | 16 | 27982 | 28063 | 16 | 28063 | 29829 |
| | | | 17 | 29829 | 29961 | 17 | 29961 | 30128 |
| | | | 18 | 30128 | 30241 | 18 | 30241 | 30330 |
| | | | 19 | 30330 | 30370 | 19 | 30370 | 30492 |
| | | | 20 | 30492 | 30577 | 20 | 30577 | 30719 |
| | | | 21 | 30719 | 30796 | 21 | 30796 | 31246 |
| | | | 22 | 31246 | 31323 | 22 | 31323 | 31693 |
| | | | 23 | 31693 | 31839 | 23 | 31839 | 31941 |
| | | | 24 | 31941 | 32056 | 24 | 32056 | 32278 |
| | | | 25 | 32278 | 32401 | 25 | 32401 | 32485 |
| | | | 26 | 32485 | 32632 | 26 | 32632 | 32996 |
| | | | 27 | 32996 | 33138 | 27 | 33138 | 33933 |
| | | | 28 | 33933 | 34028 | 28 | 34028 | 34996 |
| | | | 29 | 34996 | 35194 | 29 | 35194 | 35334 |
| | | | 30 | 35334 | 35474 | 30 | 35474 | 36563 |
| | | | 31 | 36563 | 36679 | 31 | 36679 | 36932 |
| | | | 32 | 36932 | 37165 | 32 | 37165 | 37257 |
| | | | 33 | 37257 | 37412 | 33 | 37412 | 37519 |
| | | | 34 | 37519 | 37671 | 34 | 37671 | 37969 |
| | | | 35 | 37969 | 38171 | | | |
| RTEL1-201 | 505 | 38434 | 1 | 505 | 650 | 1 | 650 | 3489 |
| ENST00000318100 | | | 2 | 3489 | 3687 | 2 | 3687 | 4041 |
| | | | 3 | 4041 | 4134 | 3 | 4134 | 4737 |
| | | | 4 | 4737 | 4818 | 4 | 4818 | 5020 |
| | | | 5 | 5020 | 5080 | 5 | 5080 | 8195 |
| | | | 6 | 8195 | 8270 | 6 | 8270 | 9660 |
| | | | 7 | 9660 | 9744 | 7 | 9744 | 14747 |
| | | | 8 | 14747 | 14812 | 8 | 14812 | 16131 |
| | | | 9 | 16131 | 16284 | 9 | 16284 | 20336 |
| | | | 10 | 20336 | 20374 | 10 | 20374 | 20459 |
| | | | 11 | 20459 | 20537 | 11 | 20537 | 22040 |
| | | | 12 | 22040 | 22137 | 12 | 22137 | 22855 |
| | | | 13 | 22855 | 22910 | 13 | 22910 | 27714 |
| | | | 14 | 27714 | 27788 | 14 | 27788 | 27982 |
| | | | 15 | 27982 | 28063 | 15 | 28063 | 29829 |
| | | | 16 | 29829 | 29961 | 16 | 29961 | 30128 |
| | | | 17 | 30128 | 30241 | 17 | 30241 | 30330 |
| | | | 18 | 30330 | 30370 | 18 | 30370 | 30492 |
| | | | 19 | 30492 | 30577 | 19 | 30577 | 30719 |
| | | | 20 | 30719 | 30796 | 20 | 30796 | 31246 |
| | | | 21 | 31246 | 31323 | 21 | 31323 | 31693 |
| | | | 22 | 31693 | 31839 | 22 | 31839 | 31941 |
| | | | 23 | 31941 | 32056 | 23 | 32056 | 32278 |
| | | | 24 | 32278 | 32401 | 24 | 32401 | 32485 |
| | | | 25 | 32485 | 32632 | 25 | 32632 | 32996 |
| | | | 26 | 32996 | 33138 | 26 | 33138 | 33933 |
| | | | 27 | 33933 | 34028 | 27 | 34028 | 34996 |
| | | | 28 | 34996 | 35194 | 28 | 35194 | 35334 |
| | | | 29 | 35334 | 35474 | 29 | 35474 | 36563 |
| | | | 30 | 36563 | 36679 | 30 | 36679 | 36932 |
| | | | 31 | 36932 | 37165 | 31 | 37165 | 37257 |
| | | | 32 | 37257 | 37412 | 32 | 37412 | 37519 |
| | | | 33 | 37519 | 37671 | 33 | 37671 | 37969 |
| | | | 34 | 37969 | 38434 | | | |
| RTEL1-202 | 551 | 16284 | 1 | 551 | 650 | 1 | 650 | 1424 |
| ENST00000356810 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4587 |
| | | | 5 | 4587 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | | | |
| RTEL1-206 | 30530 | 33067 | 1 | 30530 | 30577 | 1 | 30577 | 30719 |
| ENST00000425905 | | | 2 | 30719 | 30796 | 2 | 30796 | 31246 |
| | | | 3 | 31246 | 31323 | 3 | 31323 | 31941 |
| | | | 4 | 31941 | 32056 | 4 | 32056 | 32278 |
| | | | 5 | 32278 | 32401 | 5 | 32401 | 32485 |
| | | | 6 | 32485 | 32632 | 6 | 32632 | 32996 |
| | | | 7 | 32996 | 33067 | | | |
| RTEL1-214 | 811 | 3653 | 1 | 811 | 943 | 1 | 943 | 1424 |
| ENST00000646389 | | | 2 | 1424 | 1695 | 2 | 1695 | 3489 |
| | | | 3 | 3489 | 3653 | | | |

Suitably, the target nucleic acid encodes an RTEL1 protein, in particular mammalian RTEL1, such as human RTEL1 (See for example tables 3 and 4) which provides the pre-mRNA sequences for human and monkey, RTEL1.

In some embodiments, the target nucleic acid is selected from SEQ ID NO: 1 and/or 2 or naturally occurring variants thereof (e.g. sequences encoding a mammalian RTEL1 protein).

If employing the antisense oligonucleotide of the invention, or the conjugate of the invention, in research or diagnostics the target nucleic acid may be a cDNA or a synthetic nucleic acid derived from DNA or RNA.

For in vivo or in vitro application, the antisense oligonucleotide of the invention is typically capable of inhibiting the expression of the RTEL1 target nucleic acid in a cell, which is expressing the RTEL1 target nucleic acid. The contiguous sequence of nucleobases of the antisense oligonucleotide of the invention is typically complementary to the RTEL1 target nucleic acid, as measured across the length of the antisense oligonucleotide, optionally with the exception of one or two mismatches, and optionally excluding nucleotide based linker regions which may link the antisense oligonucleotide to an optional functional group such as a conjugate, or other non-complementary terminal nucleotides (e.g. region D′ or D″). The target nucleic acid may, in some embodiments, be a RNA or DNA, such as a messenger RNA, such as a pre-mRNA, such as human RTEL1, e.g. the human RTEL1 pre-mRNA sequence, such as that disclosed as SEQ ID NO: 1, or the cynomolgus monkey RTEL1 pre-mRNA sequence, such as that disclosed as SEQ ID NO: 2. SEQ ID NOs: 1 and 2 are DNA sequences—it will be understood that target RNA sequences have uracil (U) bases in place of the thymidine bases (T).

Further information on exemplary target nucleic acids is provided in Tables 4 and 5.

TABLE 4
Genome and assembly information for RTEL1 across species.

| | | | Genomic coordinates | | | ensembl |
|---|---|---|---|---|---|---|
| Species | Chr. | Strand | Start | End | Assembly | gene_id |
| Human | 20 | fwd | 63657810 | 63696253 | GRCh38.p12 | ENSG00000258366 |
| Cynomolgus | 10 | fwd | 95853726 | 95890939 | Macaca_fascicularis_5.0 | ENSMFAG00000043680 |
| monkey | | | | | | |

Fwd = forward strand.
The genome coordinates provide the pre-mRNA sequence (genomic sequence). The NCO reference provides the mRNA sequence (cDNA sequence).

TABLE 5
Sequence details for RTEL1 across species.

| Species | RNA type | Length (nt) | SEQ ID NO |
|---|---|---|---|
| Human | premRNA | 38444 | 1 |
| Monkey | premRNA | 37214 | 2 |

Note:
SEQ ID NO: 2 comprises regions of multiple NNNNs, where the sequencing has been unable to accurately refine the sequence, and a degenerate sequence is therefore included. For the avoidance of doubt the compounds of the invention are complementary to the actual target sequence and are not therefore degenerate compounds.

In some embodiments, the target nucleic acid is SEQ ID NO: 1.

In some embodiments, the target nucleic acid is SEQ ID NO: 2.

In some embodiments, the target nucleic acid is SEQ ID NO: 1 and 2.

### Target Sequence

The term “target sequence” as used herein refers to a sequence of nucleotides present in the target nucleic acid, which comprises the nucleobase sequence, which is complementary to the oligonucleotide of the invention. In some embodiments, the target sequence consists of a region on the target nucleic acid with a nucleobase sequence that is complementary to the contiguous nucleotide sequence of the oligonucleotide of the invention. This region of the target nucleic acid may interchangeably be referred to as the target nucleotide sequence, target sequence or target region. In some embodiments, the target sequence is longer than the complementary sequence of a single oligonucleotide, and may, for example represent a preferred region of the target nucleic acid, which may be targeted by several oligonucleotides of the invention.

The oligonucleotide of the invention comprises a contiguous nucleotide sequence, which is complementary to and hybridizes to the target nucleic acid, such as a target sequence described herein.

The target sequence to which the oligonucleotide is complementary to generally comprises a contiguous nucleobases sequence of at least 10 nucleotides. The contiguous nucleotide sequence is between 10 to 30 nucleotides in length, such as 12 to 30, such as 14 to 20, such as 15 to 18 contiguous nucleotides in length, such as 15, 16, 17 contiguous nucleotides in length.

### Target Sequence Regions

The inventors have identified particularly effective sequences of the RTEL1 target nucleic acid, which may be targeted by the oligonucleotide of the invention, or the conjugate thereof.

In some embodiments, the target sequence is SEQ ID NO 7.

In some embodiments, the target sequence is SEQ ID NO 8.

In some embodiments, the target sequence is SEQ ID NO 9.

In some embodiments, the target sequence is SEQ ID NO 10.

In some embodiments, the target sequence is SEQ ID NO 11.

SEQ ID NO 7:
TTTGACCAGAGTATGTAAAATT
SEQ ID NO 8:
ACCAGAGTATGTAAAATT
SEQ ID NO: 9:
GACCAGAGTATGTAAAATT
SEQ ID NO: 10:
TTTGACCAGAGTATGTAA
SEQ ID NO: 11:
GAGATTCAAGTTATAATAAAG

SEQ ID NOS: 7 to 11 are DNA sequences—it will be understood that target RNA sequences have uracil (U) bases in place of the thymidine bases (T).

The target sequences shown in SEQ ID NOs: 7 to 10 can be found in intron 8 of human RTEL1. The target sequence shown in SEQ ID No: 11 can be found in intron 7 of human RTEL1.

In some embodiments, the target sequence is the region from nucleotides 11753-11774 of SEQ ID NO: 1.

In some embodiments, the target sequence is the region from nucleotides 11757-11774 of SEQ ID NO: 1.

In some embodiments, the target sequence is the region from nucleotides 11756-11774 of SEQ ID NO: 1.

In some embodiments, the target sequence is the region from nucleotides 11753-11770 of SEQ ID NO: 1.

In some embodiments, the target sequence is the region from nucleotides 8681-8701 of SEQ ID NO: 1.

### Target Cell

The term a “target cell” as used herein refers to a cell, which is expressing the target nucleic acid. In some embodiments, the target cell may be in vivo or in vitro. In some embodiments, the target cell is a mammalian cell such as a rodent cell, such as a mouse cell or a rat cell, or a primate cell such as a monkey cell or a human cell.

Typically, the target cell expresses the RTEL1 mRNA, such as the RTEL1 pre-mRNA or RTEL1 mature mRNA. For experimental evaluation a target cell may be used which expresses a nucleic acid which comprises a target sequence, such as the human RTEL1 pre-mRNA, e.g. SEQ ID NO: 1.

The poly A tail of RTEL1 mRNA is typically disregarded for antisense oligonucleotide targeting.

The antisense oligonucleotide of the invention is typically capable of inhibiting the expression of the RTEL1 target nucleic acid in a cell which is expressing the RTEL1 target nucleic acid (a target cell), for example either in vivo or in vitro.

Further, the target cell may be a hepatocyte. In one embodiment, the target cell is HBV infected primary human hepatocytes, either derived from HBV infected individuals or from a HBV infected mouse with a humanized liver (PhoenixBio, PXB-mouse).

In accordance with the present invention, the target cell may be infected with HBV. Further, the target cell may comprise HBV cccDNA. Thus, the target cell preferably comprises RTEL1 mRNA, such as the RTEL1 pre-mRNA or RTEL1 mature mRNA, and HBV cccDNA.

### Naturally Occurring Variant

The term “naturally occurring variant” refers to variants of RTEL1 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian RTEL1 target nucleic acid, such as a target nucleic acid of SEQ ID NO: 1. In some embodiments, the naturally occurring variants have at least 99% homology to the human RTEL1 target nucleic acid of SEQ ID NO: 1.

### Inhibition of Expression

The term “Inhibition of expression” as used herein is to be understood as an overall term for an oligonucleotide's ability to inhibit the amount or the activity of RTEL1 in a target cell. Inhibition of activity may be determined by measuring the level of RTEL1 pre-mRNA or RTEL1 mRNA, or by measuring the level of RTEL1 or RTEL1 activity in a cell. Inhibition of expression may therefore be determined in vitro or in vivo.

Typically, inhibition of expression is determined by comparing the inhibition of activity due to the administration of an effective amount of the antisense oligonucleotide to the target cell and comparing that level to a reference level obtained from a target cell without administration of the antisense oligonucleotide (control experiment), or a known reference level (e.g. the level of expression prior to administration of the effective amount of the antisense oligonucleotide, or a predetermine or otherwise known expression level).

For example a control experiment may be an animal or person, or a target cell treated with a saline composition or a reference oligonucleotide (often a scrambled control).

The term inhibition or inhibit may also be referred as down-regulate, reduce, suppress, lessen, lower, the expression of RTEL1, such as RTEL1 pre-mRNA.

The inhibition of expression may occur e.g. by degradation of pre-mRNA or mRNA (e.g. using RNase H recruiting oligonucleotides, such as gapmers).

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (Tm). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The oligomer of the invention may comprise one or more nucleosides, which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradical capable of forming a bridge between the 2′ carbon and a second carbon in the ribose ring, such as LNA (2′-4′ biradical bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

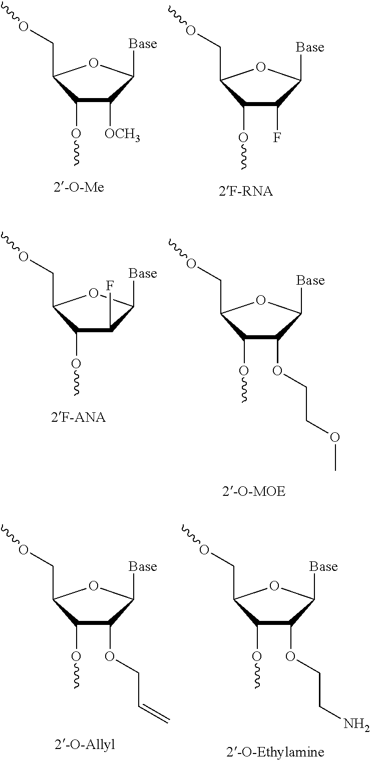

In relation to the present invention 2′ substituted sugar modified nucleosides does not include 2′ bridged nucleosides like LNA.

### Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

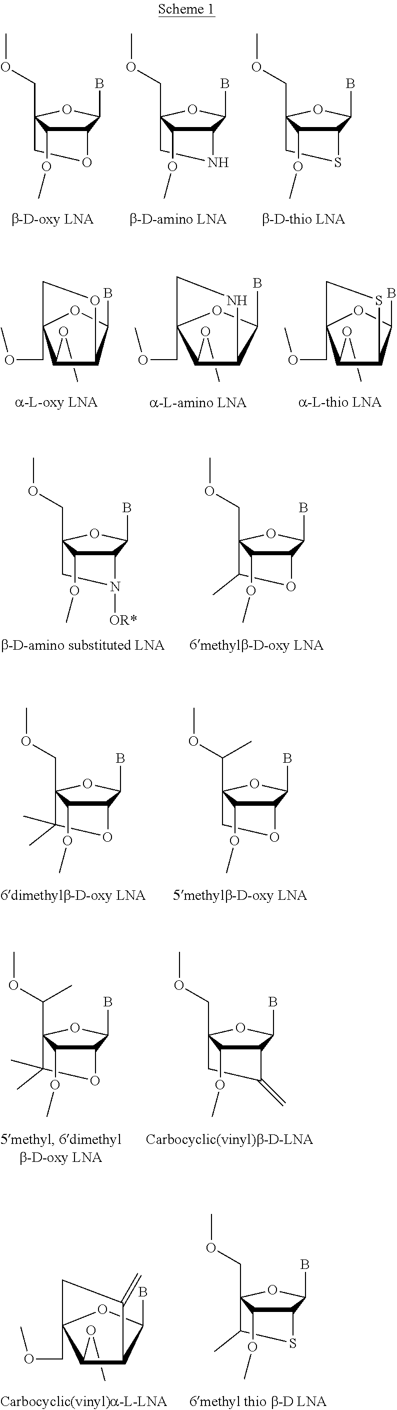

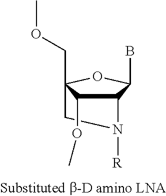

Particular LNA nucleosides are beta-D-oxy-LNA, 6′-methyl-beta-D-oxy LNA such as (S)-6′-methyl-beta-D-oxy-LNA (ScET) and ENA. A particularly advantageous LNA is beta-D-oxy-LNA.

### Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the invention are capable of recruiting a nuclease, particularly an endonuclease, preferably endoribonuclease (RNase), such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers.

### RNase H Activity and Recruitment

The RNase H activity of an antisense oligonucleotide refers to its ability to recruit RNase H when in a duplex with a complementary RNA molecule. WO01/23613 provides in vitro methods for determining RNase H activity, which may be used to determine the ability to recruit RNase H. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RNase H activity, recombinant human RNase H1 is available from Creative Biomart® (Recombinant Human RNase H1 fused with His tag expressed in E. coli).

### Gapmer

The antisense oligonucleotide of the invention, or contiguous nucleotide sequence thereof, may be a gapmer, also termed gapmer oligonucleotide or gapmer designs. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5→3’ orientation. The “gap” region (G) comprises a stretch of contiguous DNA nucleotides, which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank. In some embodiments, all internucleoside linkages between the nucleosides of the gapmer region of formula F-G-F′ are phosphorothioate internucleoside linkages.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, such as from 15 to 20, such as 16 to 18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈, or

F₁₋₈-G₁₁₋₁₆-F′₂₋₈,
with the proviso that the overall length of the gapmer regions F-G-F′ is at least 12, such as at least 14 nucleotides in length.

In an aspect of the invention the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and defines the 5′ and 3′ end of the F and F′ region, and G is a region between 6 and 16 nucleosides which are capable of recruiting RNase H.

Regions F, G and F′ are further defined below and can be incorporated into the F-G-F′ formula.

### Gapmer—Region G

Region G (gap region) of the gapmer is a region of nucleosides which enables the oligonucleotide to recruit RNase H, such as human RNase H1, typically DNA nucleosides. RNase H is a cellular enzyme, which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments, consist of 6, 7, 8, 9, 10, 11, 12, 13, 14, 15 or 16 contiguous DNA nucleosides. Further, region G may have a length of 11 to 16 contiguous DNA nucleotides.

One or more cytosine (C) DNA in the gap region may in some instances be methylated (e.g. when a DNA c is followed by a DNA g), such residues are annotated as 5-methyl-cytosine (^{me}C). In some embodiments, the gap region G may consist of 6, 7, 8, 9, 10, 11, 12, 13, 14, 15 or 16 contiguous phosphorothioate linked DNA nucleosides.

In some embodiments, all internucleoside linkages in the gap are phosphorothioate linkages.

### Gapmer—Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 2-4 contiguous nucleotides in length. In some embodiments, the length of region F is 2 contiguous nucleotides. In some embodiments, the length of region F is 3 contiguous nucleotides. In some embodiments, the length of region F is 4 contiguous nucleotides.

Advantageously the 5′ most nucleoside of region F is a sugar modified nucleoside. In some embodiments, the two 5′ most nucleosides of region F are sugar modified nucleosides. In some embodiments, the 5′ most nucleoside of region F is an LNA nucleoside. In some embodiments, the two 5′ most nucleosides of region F are LNA nucleosides.

Region F′ is 1-8 contiguous nucleotides in length, such as 2-6 contiguous nucleotides in length. In some embodiments, the length of region F′ is 2 contiguous nucleotides. In some embodiments, the length of region F′ is 4 contiguous nucleotides. In some embodiments, the length of region F′ is 5 contiguous nucleotides. In some embodiments, the length of region F′ is 6 contiguous nucleotides. Advantageously, the 3′ most nucleoside of region F′ is a sugar modified nucleoside. In some embodiments, the two 3′ most nucleosides of region F′ are sugar modified nucleosides. In some embodiments, the two 3′ most nucleosides of region F′ are LNA nucleosides. In some embodiments, the 3′ most nucleoside of region F′ is an LNA nucleoside.

It should be noted that when the length of region F is one, it is advantageously an LNA nucleoside. Further, it is noted that when the length of region F and/or F′ is two, both nucleosides of region F and/or F′ are advantageously LNA nucleosides.

In some embodiments, the sugar modified nucleosides in region F and F′ consist of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as beta-D-oxy LNA nucleosides. In an alternative embodiment, all the sugar modified nucleosides of region F and F′ are LNA nucleosides, such as beta-D-oxy LNA nucleosides, wherein region F or F′, or both regions F and F′ may comprise DNA nucleosides (an alternating flank, see definition of these for more details).

In some embodiments, the 5′ most and the 3′ most nucleosides of region F and F′ are LNA nucleosides, such as beta-D-oxy LNA nucleosides nucleosides.

In some embodiments, the internucleoside linkage between region F and region G and/or the internucleoside linkage between region F′ and region G is a phosphorothioate internucleoside linkage. In some embodiments, the internucleoside linkages between the nucleosides of region F or F′, F and F′ are phosphorothioate internucleoside linkages.

### LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments, the LNA gapmer is of formula: [LNA]₁₋₅-[region G]-[LNA]₁₋₅, wherein region G is or comprises a region of contiguous DNA nucleosides which are capable of recruiting RNaseH.

### MOE Gapmers

A MOE gapmers is a gapmer wherein regions F and F′ consist of MOE nucleosides. In some embodiments, the MOE gapmer is of design [MOE]₁₋₈-[Region G]₅₋₁₆-[MOE]₁₋₈, such as [MOE]₂₋₇-[Region G]₆₋₁₄-[MOE]₂₋₇, such as [MOE]₃₋₆-[Region G]₈₋₁₂-[MOE]₃₋₆, wherein region G is as defined in the Gapmer definition. MOE gapmers with a 5-10-5 design (MOE-DNA-MOE) have been widely used in the art.

### Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleoside. In some embodiments, wherein at least one of region F and F′, or both region F and F′ comprise at least one LNA nucleoside, the remaining nucleosides of region F and F′ are independently selected from the group consisting of MOE and LNA. In some embodiments, wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

### Alternating Flank Gapmers

Flanking regions may comprise both LNA and DNA nucleoside and are referred to as “alternating flanks” as they comprise an alternating motif of LNA-DNA-LNA nucleosides. Gapmers comprising at least one alternating flank are referred to as “alternating flank gapmers”. “Alternative flank gapmers” are thus LNA gapmer oligonucleotides where at least one of the flanks (F or F′) comprises DNA in addition to the LNA nucleoside(s). In some embodiments, at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides. Alternating flank LNA gapmers are disclosed in WO2016/127002.

An alternating flank region may comprise up to 3 contiguous DNA nucleosides, such as 1 to 2 or 1 or 2 or 3 contiguous DNA nucleosides.

The alternating flak regions can be annotated as a series of integers, representing a number of LNA nucleosides (L) followed by a number of DNA nucleosides (D), for example [L]1-3-[D]1-3-[L]1-3 or [L]1-2-[D]1-2-[L]1-2-[D]1-2-[L]1-2 In oligonucleotide designs these will often be represented as numbers such that 2-2-1 represents 5′ [L]2-[D]2-[L] 3′, and 1-1-1-1-1 represents 5′ [L]-[D]-[L]-[D]-[L]3′. The length of the flank (region F and F′) in oligonucleotides with alternating flanks may be as described herein above for these regions, such as 4 to 8, such as 5 to 6 nucleosides, such as 4, 5, 6 or 7 modified nucleosides. It may be advantageous to have at least two LNA nucleosides at the 3′ end of the 3′ flank (F′), to confer additional exonuclease resistance.

### Region D′ or D″ in an Oligonucleotide

The oligonucleotide of the invention may in some embodiments comprise or consist of the contiguous nucleotide sequence of the oligonucleotide, which is complementary to the target nucleic acid, such as a gapmer region F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonuclease protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments, the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃—F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments, the internucleoside linkage positioned between region D′ and region F is a phosphodiester linkage. In some embodiments, the internucleoside linkage positioned between region F′ and region D″ is a phosphodiester linkage.

### Conjugate

The term conjugate as used herein refers to an oligonucleotide, which is covalently linked to a non-nucleotide moiety (conjugate moiety or region C or third region). The conjugate moiety may be covalently linked to the antisense oligonucleotide, optionally via a linker group, such as region D′ or D″.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GaINAc), cell surface receptor ligands, drug substances, hormones, lipophilic substances, polymers, proteins, peptides, toxins (e.g. bacterial toxins), vitamins, viral proteins (e.g. capsids) or combinations thereof.

Exemplary conjugate moieties include those capable of binding to the asialoglycoprotein receptor (ASGPR). In particular, tri-valent N-acetylgalactosamine conjugate moieties are suitable for binding to the ASGPR, see for example WO 2014/076196, WO 2014/207232 and WO 2014/179620. Such conjugates serve to enhance uptake of the oligonucleotide to the liver.

In some embodiments, the conjugate is an antibody or an antibody fragment which has a specific affinity for a transferrin receptor, for example as disclosed in WO 2012/143379 herby incorporated by reference. In some embodiments, the non-nucleotide moiety is an antibody or antibody fragment, such as an antibody or antibody fragment that facilitates delivery across the blood-brain-barrier, in particular an antibody or antibody fragment targeting the transferrin receptor.

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments, of the invention the conjugate or oligonucleotide conjugate of the invention may optionally, comprise a linker region (second region or region B and/or region Y), which is positioned between the oligonucleotide or contiguous nucleotide sequence complementary to the target nucleic acid (region A or first region) and the conjugate moiety (region C or third region).

Biocleavable linkers (Region B) comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment, the biocleavable linker is susceptible to S1 nuclease cleavage. In some embodiments, the physiologically labile linker (biocleavable) comprises between 1 and 10 linked nucleosides, such as 1, 2, 3, 4, 5, 6, 7, 8, 9 or 10 linked nucleosides, such as between 2 and 6 linked nucleosides, such as between 2 and 5 linked nucleosides, such as between 2 and 4 linked nucleosides, where at least two consecutive linkages are biocleavable, such as phosphodiester linkages, such as at least 3 or 4 or 5 consecutive phosphodiester linkages. Preferably the nucleosides are DNA or RNA.

In one embodiment, the linker between the oligonucleotide and the conjugate moiety is a physiologically labile linker composed of 2 to 5 consecutive phosphodiester linked nucleosides comprising at least two consecutive phosphodiester linkages at the 5′ or 3′ terminal of the contiguous nucleotide sequence of the antisense oligonucleotide.

In some embodiments, the physiologically labile linker comprises or consists of a DNA dinucleotide with a sequence selected from the group consisting of AA, AT, AC, AG, TA, TT, TC, TG, CA, CT, CC, CG, GA, GT, GC, or GG, where there is a phosphodiester linkage between the two DNA nucleosides and at least one further phosphodiester at the 5′ or 3′ end of the dinucleotide linking either the oligonucleotide of the nucleic acid molecule to the dinucleotide or the conjugate moiety to the dinucleotide. For example, the linker may by a CA dinucleotide. In some embodiments, the physiologically labile linker comprises or consists of a DNA trinucleotide of sequence AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TAG, TCA, TCT, TCC, TCG, TGA, TGT, TGC, TGG, CAA, CAT, CAC, CAG, CTA, CTG, CTC, CTT, CCA, CCT, CCC, CCG, CGA, CGT, CGC, CGG, GAA, GAT, GAC, CAG, GTA, GTT, GTC, GTG, GCA, GCT, GCC, GCG, GGA, GGT, GGC, or GGG, where there are phosphodiester linkages between the DNA nucleosides and potentially a further phosphodiester at the 5′ or 3′ end of the trinucleotide. Phosphodiester containing biocleavable linkers are described in more detail in WO 2014/076195 (hereby incorporated by reference). In a conjugate compound with a biocleavable linker at least about 50% of the conjugate moiety is cleaved from the oligonucleotide, such as at least about 60% cleaved, such as at least about 70% cleaved, such as at least about 80% cleaved, such as at least about 85% cleaved, such as at least about 90% cleaved, such as at least about 95% of the conjugate moiety is cleaved from the oligonucleotide cleaved when compared against a standard.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide conjugates of the present invention can be constructed of the following regional elements A-C, A-B—C, A-B—Y—C, A-Y—B—C or A-Y—C. In some embodiments, the linker (region Y) is an amino alkyl, such as a C2-C36 amino alkyl group, including, for example C6 to C12 amino alkyl groups. In some embodiments, the linker (region Y) is a C6 amino alkyl group.

Preferably, the cleavable linker is cleaved after delivery of the conjugate of the present invention to its target cell. For example, the linker is cleaved off after GaINAc mediated delivery of the conjugate to the target cell (such as a liver cell), leaving the naked compound as active drug. For example, the conjugates tested in the Examples section contain a CA dinucleotide linker.

### Pharmaceutically Acceptable Salts

The term “pharmaceutically acceptable salts” refers to those salts, which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compounds of the present invention can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. Prophylactic can be understood as preventing an HBV infection from turning into a chronic HBV infection or the prevention of severe liver diseases such as liver cirrhosis and hepatocellular carcinoma caused by a chronic HBV infection.

### Prevention

Herein the term “preventing”, “prevention” or “prevents” relates to a prophylactic treatment, i.e. to a measure or procedure the purpose of which is to prevent, rather than to cure a disease. Prevention means that a desired pharmacological and/or physiological effect is obtained that is prophylactic in terms of completely or partially preventing a disease or symptom thereof. Accordingly, herein “preventing a HBV infection” includes preventing a HBV infection from occurring in a subject, and preventing the occurrence of symptoms of a HBV infection. In the present invention in particular the prevention of HBV infection in children from HBV infected mothers are contemplated. Also contemplated is the prevention of an acute HBV infection turning into a chronic HBV infection.

### Patient

For the purposes of the present invention, the “subject” or “patient” may be a vertebrate. In context of the present invention, the term “subject” includes both humans and other animals, particularly mammals, and other organisms. Thus, the herein provided means and methods are applicable to both human therapy and veterinary applications. Accordingly, herein the subject may be an animal such as a mouse, rat, hamster, rabbit, guinea pig, ferret, cat, dog, chicken, sheep, bovine species, horse, camel, or primate. Preferably, the subject is a mammal. More preferably, the subject is human. In some embodiments, the patient is suffering from a disease as referred to herein, such as HBV infection. In some embodiments, the patient is susceptible to said disease.

### DETAILED DESCRIPTION OF THE INVENTION

Hepatitis B virus (HBV) covalently closed circular DNA (cccDNA) in infected hepatocytes is responsible for persistent chronic infection and reactivation, being the template for all viral subgenomic transcripts and pre-genomic RNA (pgRNA) to ensure both newly synthesized viral progeny and cccDNA pool replenishment via intracellular nucleocapsid recycling. RTEL1 is associated with cccDNA stability. Inhibition of RTEL1 leads to destabilization of cccDNA in HBV infected subjects, which in turn opens the opportunity for a complete cure of chronically infected HBV patients.

One aspect of the present invention is an enhanced antisense oligonucleotide targeting RTEL1, or a conjugate thereof for use in the treatment and/or prevention of HBV infection, in particular a chronic HBV infection.

In one embodiment, the antisense oligonucleotide targeting RTEL1, or a conjugate thereof can for example reduce the expression of RTEL1 protein, the binding of RTEL1 protein to cccDNA and thereby reducing cccDNA and/or pgRNA in an infected cell, such as an HBV infected cell.

In one embodiment, the antisense oligonucleotide targeting RTEL1, or a conjugate thereof is capable of reducing HBsAg and/or HBeAg in vivo in an HBV infected individual.

In one embodiment, the antisense oligonucleotide targeting RTEL1, or a conjugate thereof is capable of reducing cccDNA in vivo in an HBV infected individual.

In one embodiment, the antisense oligonucleotide targeting RTEL1, or a conjugate thereof is capable of reducing pgRNA in vivo in an HBV infected individual.

### The Antisense Oligonucleotide of the Invention

The enhanced antisense oligonucleotides of the invention or conjugates thereof are potentially excellent RTEL1 inhibitors since they can target the RTEL1 transcript and promote its degradation either via RNase H cleavage.

One aspect of the present invention is an enhanced antisense oligonucleotide or conjugates thereof for use in treatment and/or prevention of HBV infection.

The present section describes novel oligonucleotides suitable for use in treatment and/or prevention of HBV infection.

The antisense oligonucleotides of the present invention or conjugates thereof are capable of inhibiting expression of RTEL1 in vitro and in vivo. The inhibition is achieved by hybridizing an oligonucleotide to a target nucleic acid encoding RTEL1 or which is involved in the regulation of RTEL1. The target nucleic acid may be a mammalian RTEL1 sequence, such as the sequence of SEQ ID NO: 1 and/or 2.

In some embodiments, the antisense oligonucleotide of the invention or conjugates thereof capable of modulating the expression of the target by inhibiting or down-regulating it. Preferably, such modulation produces an inhibition of expression of at least 20% compared to the normal expression level of the target, more preferably at least 30%, at least 40%, at least 50%, at least 60%, at least 70%, at least 80%, or at least 90% inhibition compared to the normal expression level of the target. In some embodiments, the antisense oligonucleotide of the invention or conjugates thereof may be capable of inhibiting expression levels of RTEL1 mRNA by at least 60% or 70% in vitro using 10 μM in PXB-PHH cells. In some embodiments, the antisense oligonucleotide of the invention or conjugates thereof may be capable of inhibiting expression levels of RTEL1 protein by at least 50% in vitro using 10 μM in PXB-PHH cells, this range of target reduction is advantageous in terms of selecting antisense oligonucleotides with good correlation to the cccDNA reduction. Suitably, the examples provide assays, which may be used to measure RTEL1 RNA inhibition (e.g. Example 1). The target inhibition is triggered by the hybridization between a contiguous nucleotide sequence of the antisense oligonucleotide and the target nucleic acid. In some embodiments, the antisense oligonucleotide of the invention comprises mismatches between the antisense oligonucleotide and the target nucleic acid. Despite mismatches hybridization to the target nucleic acid may still be sufficient to show a desired inhibition of RTEL1 expression. Reduced binding affinity resulting from mismatches may advantageously be compensated by increased number of nucleotides in the oligonucleotide and/or an increased number of modified nucleosides capable of increasing the binding affinity to the target, such as 2′ sugar modified nucleosides, including LNA, present within the oligonucleotide sequence.

An aspect of the present invention relates to an enhanced antisense oligonucleotide of 12 to 60 nucleotides in length, which comprises a contiguous nucleotide sequence of at least 10 nucleotides in length, such as at least 12 to 30 nucleotides in length, which is at least 95% complementary, such as fully complementary, to a mammalian RTEL1 target nucleic acid, in particular a human RTEL1 nucleic acid. The antisense oligonucleotide is capable of inhibiting the expression of RTEL1.

In some embodiments, the antisense oligonucleotide of the present invention comprises a contiguous nucleotide sequence of 12 to 22 nucleotides, such as of 15 to 20 nucleotides, with at least 90% complementarity, such as fully complementary, to the target nucleic acid of SEQ ID NO: 7.

In some embodiments, antisense oligonucleotide comprises a contiguous nucleotide sequence of 15 to 18 nucleotides, such as of 17 or 18 nucleotides, with at least 90% complementarity, such as fully complementary, to the target nucleic acid of SEQ ID NO: 8.

In some embodiments, antisense oligonucleotide comprises a contiguous nucleotide sequence of 15 to 19 nucleotides, such as of 18 or 19 nucleotides, with at least 90% complementarity, such as fully complementary, to the target nucleic acid of SEQ ID NO: 9.

In some embodiments, antisense oligonucleotide comprises a contiguous nucleotide sequence of 15 to 18 nucleotides, such as of 17 or 18 nucleotides, with at least 90% complementarity, such as fully complementary, to the target nucleic acid of SEQ ID NO: 10.

In some embodiments, the antisense oligonucleotide of the present invention comprises a contiguous nucleotide sequence of 12 to 22 nucleotides, such as of 17 to 22 nucleotides, with at least 90% complementarity, such as fully complementary, to the target nucleic acid of SEQ ID NO: 11.

In some embodiments, the antisense oligonucleotide comprises a contiguous nucleotide sequence of 15 to 22 nucleotides, such as of 15 to 18 nucleotides, such as of 17 or 18 nucleotides with at least 90% complementarity, such as fully complementary, to the target nucleic acid selected from the following regions of SEQ ID NO: 1: 8681-8701 of SEQ ID NO: 1, 11753-11774 of SEQ ID NO: 1, such as to a region from nucleotides 11757-11774, 11756-11774, or 11753-11770 of SEQ ID NO: 1.

An aspect of the invention relates to an antisense oligonucleotide, which is an antisense oligonucleotide of 12 to 30 nucleotides in length, comprising a contiguous nucleotide sequence of at least 10 nucleotides, such as 10 to 30 nucleotides in length, which is at least 90% complementary, such as fully complementary, to a mammalian RTEL1.

A further aspect of the present invention relates to an antisense oligonucleotide comprising a contiguous nucleotide sequence of 12 to 20, such as 15 to 22, nucleotides in length with at least 90% complementarity, such as fully complementary, to the target nucleic acid of SEQ ID NO: 1.

In some embodiments, the antisense oligonucleotide comprises a contiguous sequence of 10 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

It is advantageous if the antisense oligonucleotide of the invention, or contiguous nucleotide sequence thereof is fully complementary (100% complementary) to a region of the target nucleic acid, or in some embodiments, may comprise one or two mismatches between the oligonucleotide and the target nucleic acid.

In some embodiments, the antisense oligonucleotide sequence is 100% complementary to a corresponding target nucleic acid of SEQ ID NO: 1.

In some embodiments, the antisense oligonucleotide or the contiguous nucleotide sequence of the invention is at least 95% complementarity, such as fully (or 100%) complementary, to the target nucleic acid of SEQ ID NO: 1 and SEQ ID NO: 2.

In some embodiments, the contiguous nucleotide sequence comprises a sequence of nucleobases selected from the group consisting of SEQ ID NO: 3, 4, 5 and 6, or at least 14 contiguous nucleotides thereof.

In some embodiments, the antisense oligonucleotide of the invention or contiguous nucleotide sequence thereof, comprises or consists of 10 to 30 nucleotides in length, such as from 12 to 25, such as 11 to 22, such as from 12 to 20, such as from 14 to 18 or 14 to 16 contiguous nucleotides in length.

In some embodiments, the antisense oligonucleotide or contiguous nucleotide sequence thereof comprises or consists of 22 or less nucleotides, such as 20 or less nucleotides, such as 18 or less nucleotides, such as 14, 15, 16 or 17 nucleotides. It is to be understood that any range given herein includes the range endpoints. Accordingly, if an oligonucleotide is said to include from 10 to 30 nucleotides, both 10 and 30 nucleotides are included.

In some embodiments, the contiguous nucleotide sequence comprises or consists of 10, 11, 12, 13, 14, 15, 16, 17, 18, 19, 20, 21 or 22 contiguous nucleotides in length.

In some embodiments, the antisense oligonucleotide or contiguous nucleotide sequence thereof comprises or consists of a sequence selected from SEQ ID NO: 3, 4, 5 and 6.

The invention provides antisense oligonucleotides according to the invention, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 7.

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 8.

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 9.

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 10.

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 11.

In advantageous embodiments, the antisense oligonucleotide comprises one or more sugar modified nucleosides, such as one or more 2′ sugar modified nucleosides, such as one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In some embodiments, the contiguous nucleotide sequence comprises LNA nucleosides.

In some embodiments, of the oligonucleotide of the present invention, all LNA nucleosides are beta-D-oxy LNA nucleosides.

In some embodiments, the contiguous nucleotide sequence comprises LNA nucleosides and DNA nucleosides.

In some embodiments, the contiguous nucleotide sequence comprises 2′-O-methoxyethyl (2′MOE) nucleosides.

In some embodiments, the contiguous nucleotide sequence comprises 2′-O-methoxyethyl (2′MOE) nucleosides and DNA nucleosides.

Advantageously, the 3′ most nucleoside of the antisense oligonucleotide, or contiguous nucleotide sequence thereof is a 2′ sugar modified nucleoside.

Advantageously, the antisense oligonucleotide comprises at least one modified internucleoside linkage, such as phosphorothioate or phosphorodithioate.

In some embodiments, the at least one internucleoside linkage in the contiguous nucleotide sequence is a phosphorothioate internucleoside linkages.

In some embodiments, at least one internucleoside linkage in the contiguous nucleotide sequence is a phosphorodithioate internucleoside linkages.

In some embodiments, at least one internucleoside linkage in the contiguous nucleotide sequence is a phosphodiester internucleoside linkages.

In some embodiments, all the internucleoside linkages within the contiguous nucleotide sequence are phosphorothioate internucleoside linkages.

In some embodiments, at least 75% the internucleoside linkages within the antisense oligonucleotide, or contiguous nucleotide sequence thereof, are phosphorothioate internucleoside linkages.

In some embodiments, all the internucleoside linkages within the antisense oligonucleotide, or contiguous nucleotide sequence thereof, are phosphorothioate internucleoside linkages.

In an advantageous embodiment of the invention the antisense oligonucleotide of the invention is capable of recruiting RNase H, such as RNase H1. In some embodiments, the antisense oligonucleotide of the invention, or the contiguous nucleotide sequence thereof is a gapmer.

In some embodiments, the antisense oligonucleotide, or contiguous nucleotide sequence thereof, consists or comprises a gapmer of formula 5′-F-G-F′-3′.

In some embodiments, region G consists of 6-16 DNA nucleoside, such as 11 to 16 DNA nucleosides. In some embodiments, region F comprises 2 to 4 DNA nucleosides and/or region F′ comprises DNA 2 to 6 nucleosides.

In some embodiments, region F and F′ each comprise at least one LNA nucleoside.

In some embodiments, the oligonucleotide of the present invention is a LNA gapmer with uniform flanks. For example, the LNA gapmer with uniform flanks may have a design selected from the following designs: 4-12-2, 2-13-4 and 2-12-5. Table 6 lists preferred designs for each motif sequence.

In some embodiments, of the invention, the LNA gapmer is an alternating flank LNA gapmer. In some embodiments, the alternating flank LNA gapmer comprises at least one alternating flank (such as flank F′). In some embodiments, the alternating flank LNA gapmer comprises one alternating flank (such as flank F′) and one uniform flank (such as flank F). For example, the LNA gapmer with one alternating F′ flank may have the following design: 2-11-1-2-1-1-3.

The invention provides the following oligonucleotide compounds (Table 6):

TABLE 6
list of oligonucleotide motif sequences
of the invention (indicated by SEQ ID NO),
designs of these, as well as specific
oligonucleotide compounds of the
invention (indicated by CMP ID NO)
designed based on the motif sequence.

| | | position | | | | Oligo- |
|---|---|---|---|---|---|---|
| SEQ | | on SEQ ID | | | CMP | nucleo- |
| ID | Motif | NO: 1 | | | ID | tide |
| NO | sequence | Start | end | Design | NO | Compound |
| 3 | AATTTTA | 11757 | 11774 | 4-12- | 3_1 | AATTtta |
| | CATACTC | | | 2 | | catactc |
| | TGGT | | | | | tgGT |
| 4 | AATTTTA | 11756 | 11774 | 2-13- | 4_1 | AAtttta |
| | CATACTC | | | 4 | | catact |
| | TGGTC | | | | | ctGGTC |
| 5 | TTACATA | 11753 | 11770 | 2-12- | 5_1 | TTacata |
| | CTCTGGT | | | 5 | | ctctggt |
| | CAAA | | | | | CAAA |
| 6 | CTTTATT | 8681 | 8701 | 2-11- | 6_1 | CTttatt |
| | ATAACTT | | | 1-2- | | ataact |
| | GAATCTC | | | 1-1-3 | | TgaAtC |
| | | | | | | TC |

The heading “Oligonucleotide compound” in the table represents specific designs of a motif sequence. Capital letters are beta-D-oxy LNA nucleosides, lowercase letters are DNA nucleosides, all LNA C are 5-methyl cytosine, all internucleoside linkages are phosphorothioate internucleoside linkages. The heading “Designs” refers to the gapmer design, F-G-F′. In classic gapmer design, i.e. gapmers with uniform flanks (e.g. 4-12-2), all the nucleotides in the flanks (F and F′) are constituted of the same type of 2′-sugar modified nucleoside, e.g. LNA, cET, or MOE, and a stretch of DNA in the middle forming the gap (G). In gapmers with alternating flank designs, the flanks of oligonucleotide are annotated as a series of integers, representing a number of beta-D-oxy LNA nucleosides (L) followed by a number of DNA nucleosides (D). For example, a flank F′ with a 1-2-1-1-3 motif represents LDDLDLLL(see CMP ID NO 6_1). Both flanks have a beta-D-oxy LNA nucleoside at the 5′ and 3′ terminal. The gap region (G), which is constituted of a number of DNA nucleosides is located between the flanks.

For some embodiments of the invention, the oligonucleotide is selected from the group of oligonucleotide compounds consisting of CMP-ID-NO: 3_1, 4_1, 5_1 and 6_1 (see Table 6).

In all instances, the F-G-F′ design may further include region D′ and/or D″ as described in the “Definitions” section under “Region D′ or D″ in an oligonucleotide”. In some embodiments, the oligonucleotide of the invention has 1, 2 or 3 phosphodiester linked nucleoside units, such as DNA units, at the 5′ or 3′ end, such as at the 5′ end, of the gapmer region. In some embodiments, the oligonucleotide of the invention consists of two 5′ phosphodiester linked DNA nucleosides followed by a F-G-F′ gapmer region as defined above. Oligonucleotides that contain phosphodiester linked DNA units at the 5′ or 3′ end are suitable for conjugation and may further comprise a conjugate moiety as described herein. For delivery to the liver ASGPR targeting moieties are particular advantageous as conjugate moieties, see the Conjugate section for further details.

### Conjugates

Since HBV infection primarily affects the hepatocytes in the liver it is advantageous to conjugate the enhanced antisense oligonucleotide of the invention to a conjugate moiety that will increase the delivery of the antisense oligonucleotide to the liver compared to the unconjugated antisense oligonucleotide. In one embodiment, liver targeting moieties are selected from moieties comprising cholesterol or other lipids or conjugate moieties capable of binding to the asialoglycoprotein receptor (ASGPR).

In some embodiments, the invention provides a conjugate comprising an antisense oligonucleotide of the invention covalently attached to a conjugate moiety.

The asialoglycoprotein receptor (ASGPR) conjugate moiety comprises one or more carbohydrate moieties capable of binding to the asialoglycoprotein receptor (ASPGR targeting moieties) with affinity equal to or greater than that of galactose. The affinities of numerous galactose derivatives for the asialoglycoprotein receptor have been studied (see for example: Jobst, S. T. and Drickamer, K. J B. C. 1996, 271, 6686) or are readily determined using methods typical in the art.

In one embodiment, the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine. Advantageously the asialoglycoprotein receptor targeting moiety is N-acetylgalactosamine (GaINAc).

To generate the ASGPR conjugate moiety the ASPGR targeting moieties (preferably GaINAc) can be attached to a conjugate scaffold. Generally, the ASPGR targeting moieties can be at the same end of the scaffold. In one embodiment, the conjugate moiety consists of two to four terminal GaINAc moieties linked to a spacer, which links each GaINAc moiety to a brancher molecule that can be conjugated to the antisense oligonucleotide.

In a further embodiment, the conjugate moiety is mono-valent, di-valent, tri-valent or tetra-valent with respect to asialoglycoprotein receptor targeting moieties. Advantageously the asialoglycoprotein receptor targeting moiety comprises N-acetylgalactosamine (GaINAc) moieties.

GaINAc conjugate moieties can include, for example, those described in WO 2014/179620 and WO 2016/055601 and PCT/EP2017/059080 (hereby incorporated by reference), as well as small peptides with GaINAc moieties attached such as Tyr-Glu-Glu-(aminohexyl GaINAc)3 (YEE(ahGaINAc)₃; a glycotripeptide that binds to asialoglycoprotein receptor on hepatocytes, see, e.g., Duff, et al., Methods Enzymol, 2000, 313, 297); lysine-based galactose clusters (e.g., L3G4; Biessen, et al., Cardovasc. Med., 1999, 214); and cholane-based galactose clusters (e.g., carbohydrate recognition motif for asialoglycoprotein receptor).

The ASGPR conjugate moiety, in particular a trivalent GaINAc conjugate moiety, may be attached to the 3′- or 5-end of the oligonucleotide using methods known in the art. In one embodiment, the ASGPR conjugate moiety is linked to the 5′-end of the oligonucleotide.

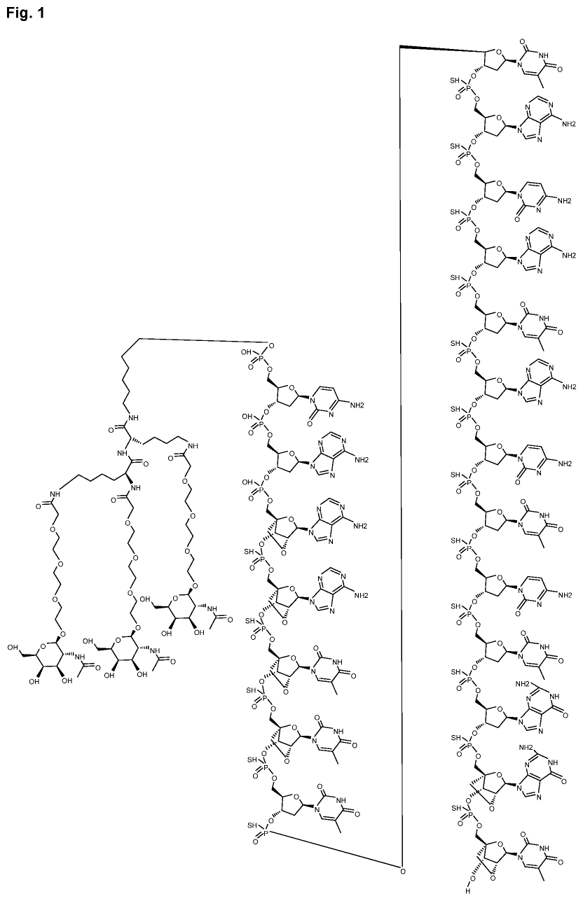

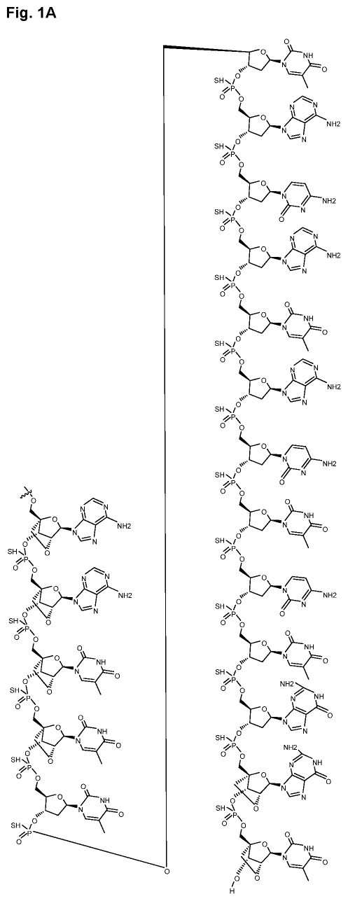

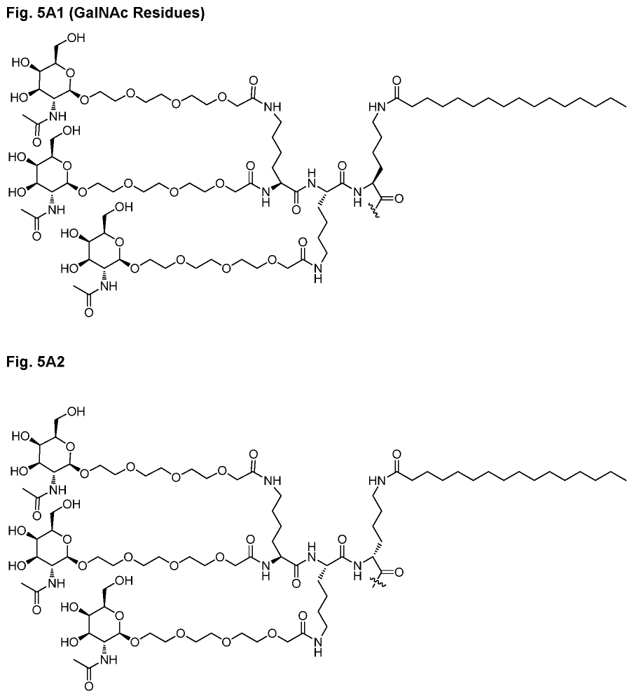

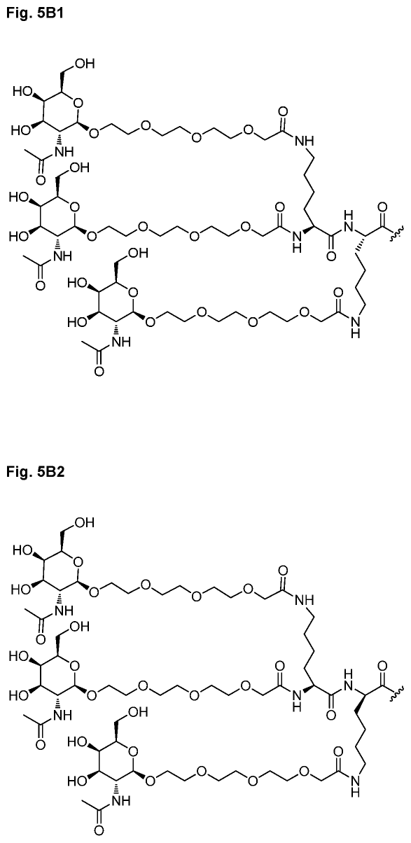

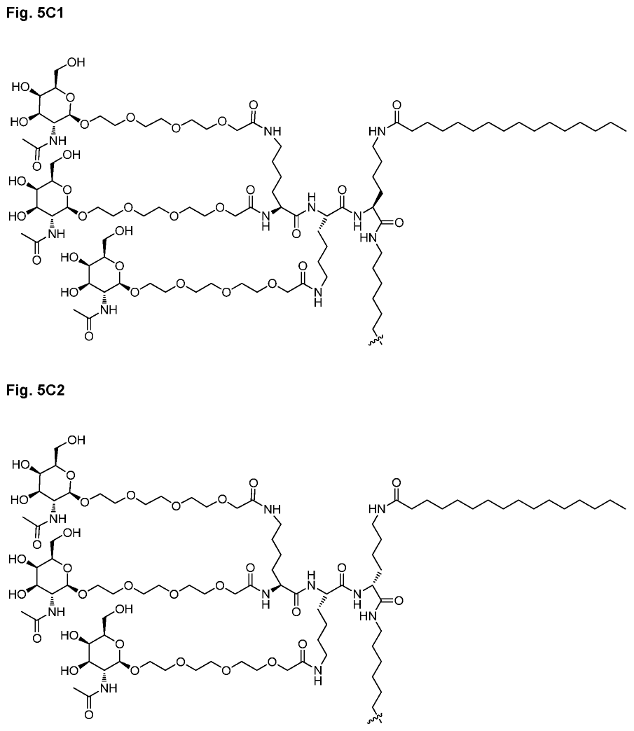

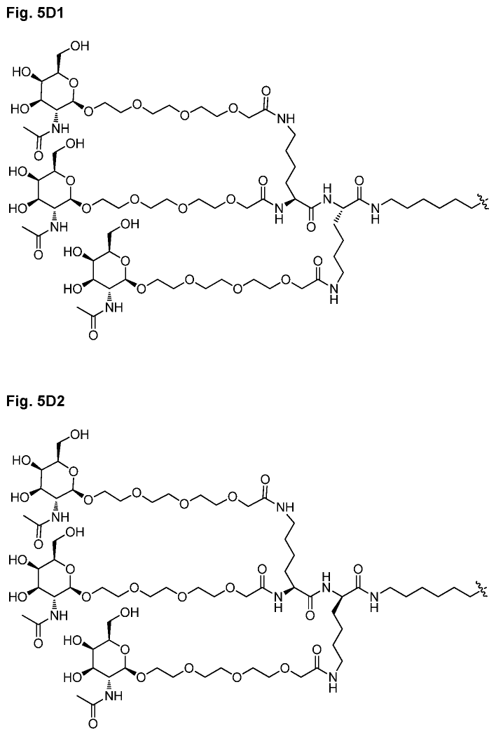

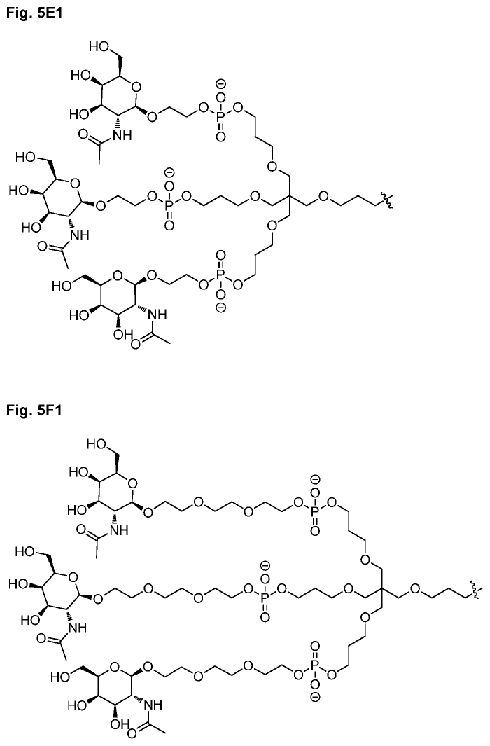

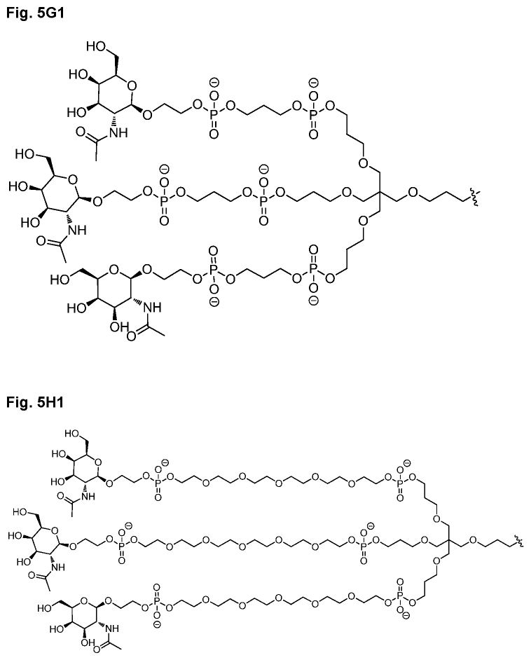

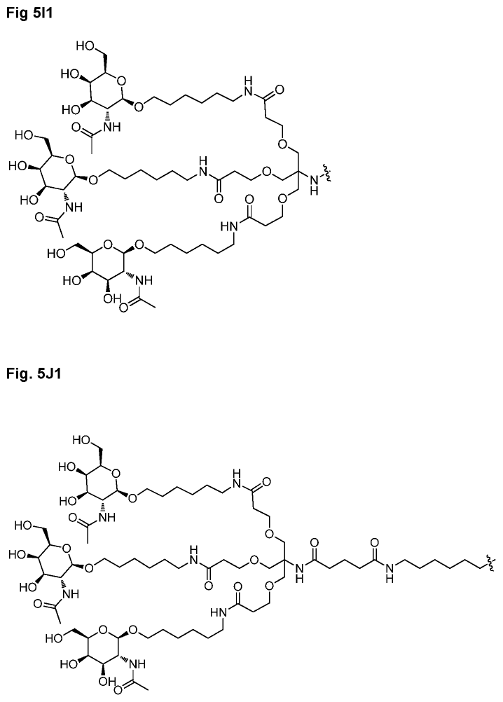

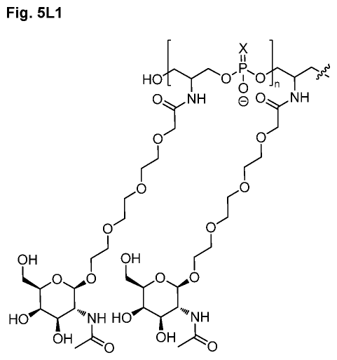

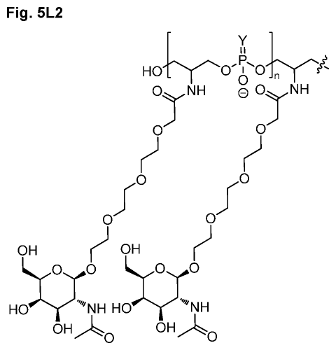

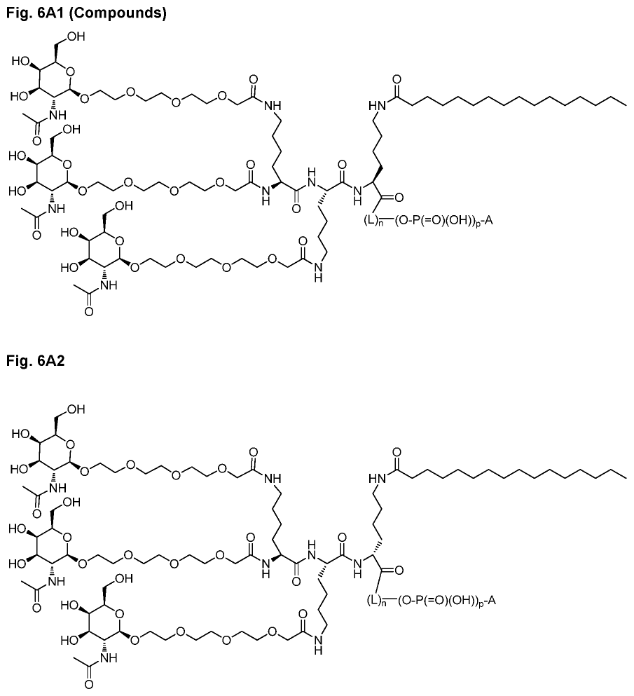

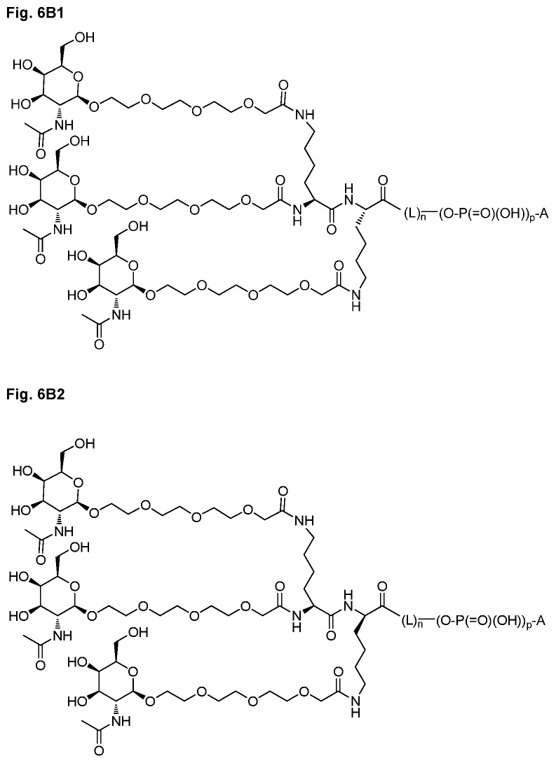

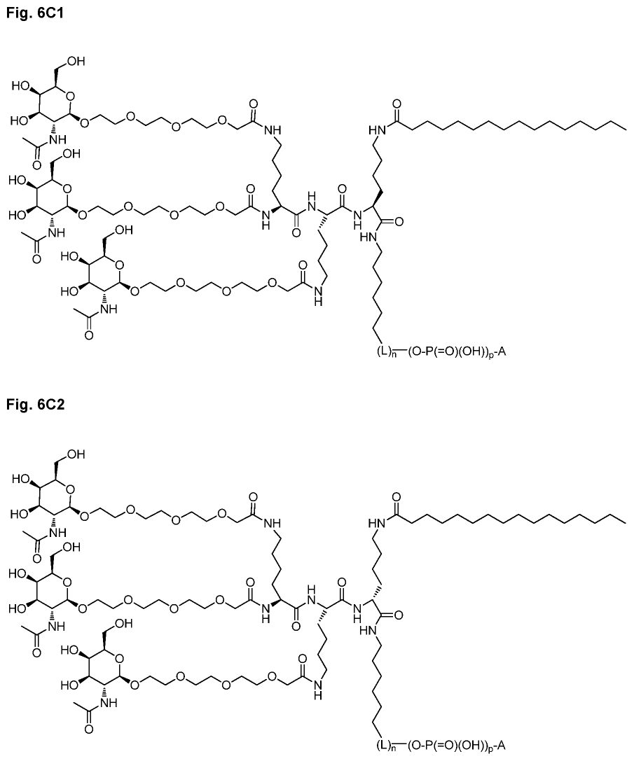

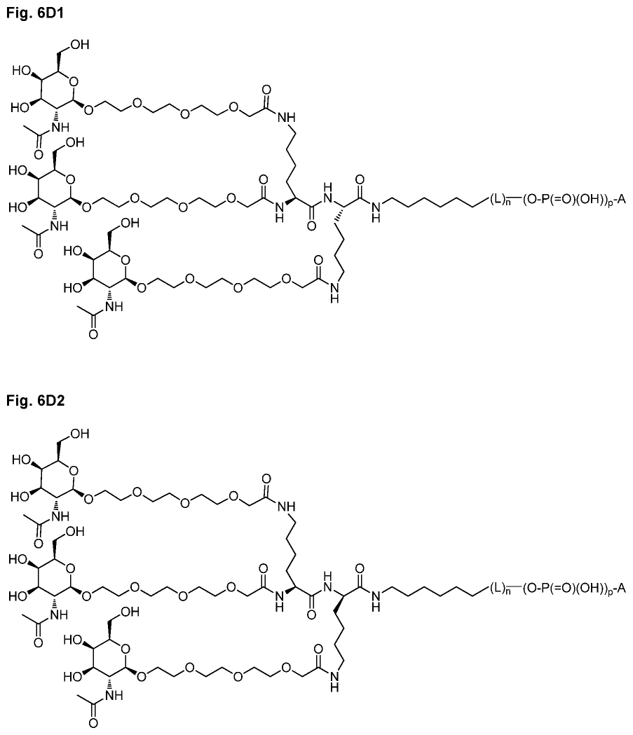

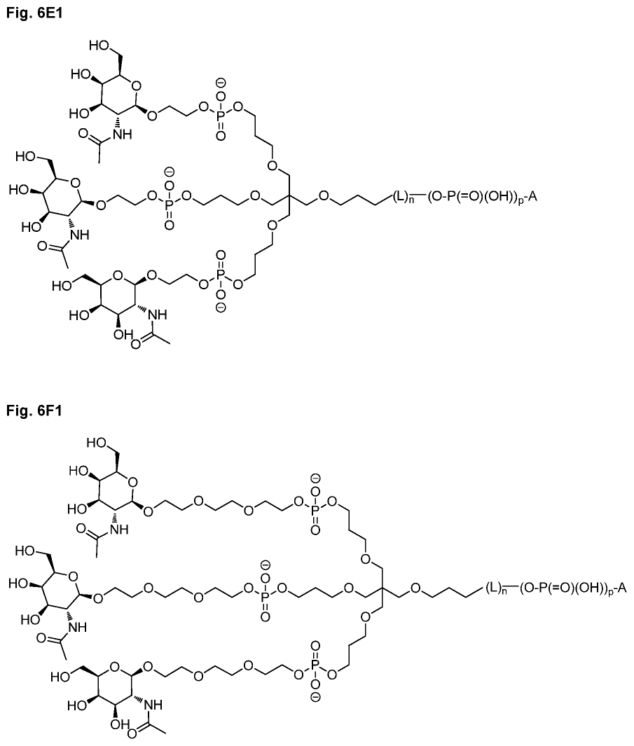

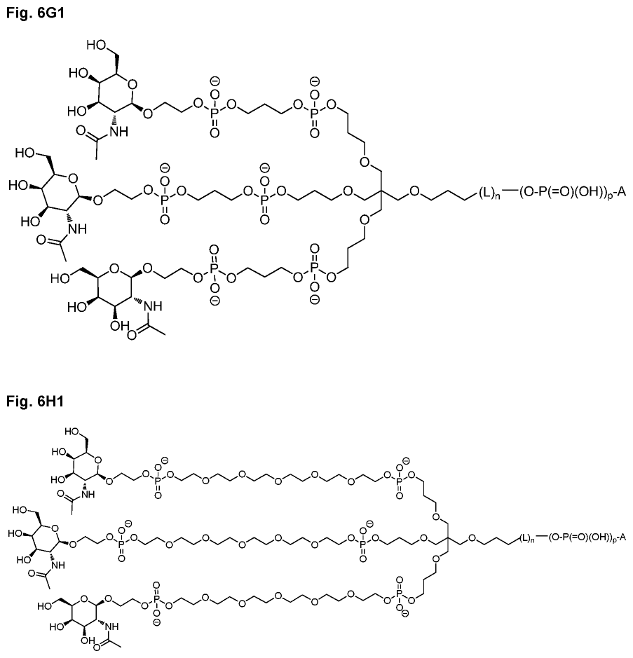

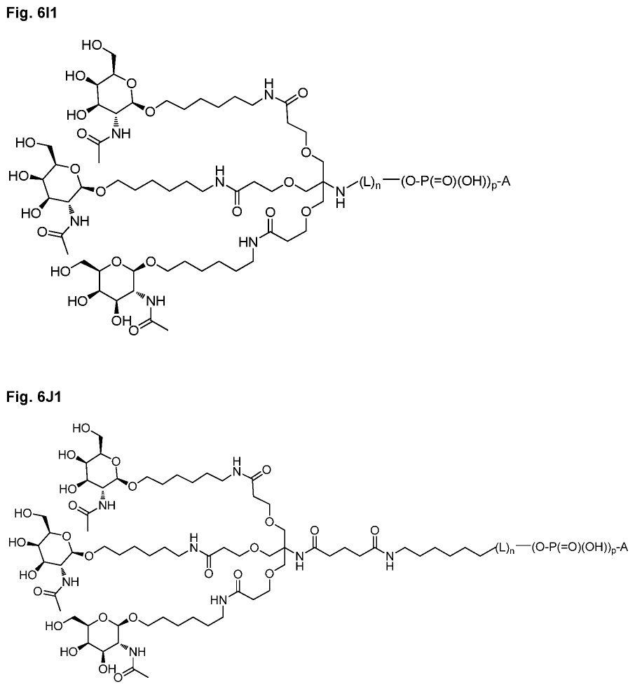

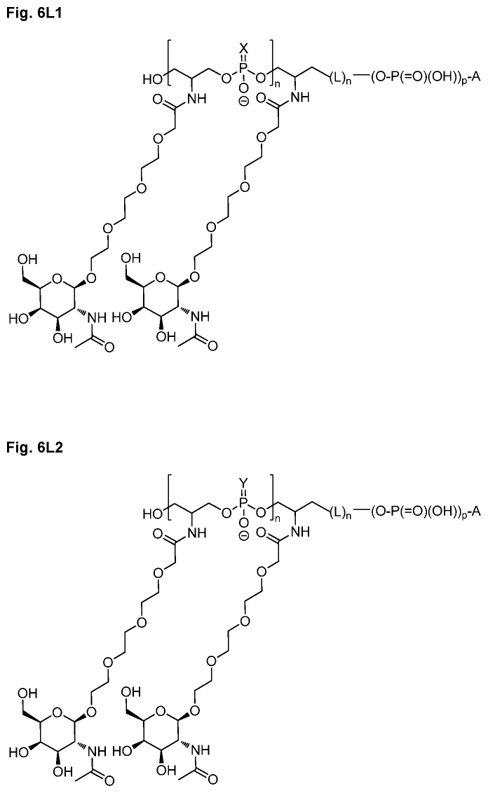

In one embodiment, the conjugate moiety is a tri-valent N-acetylgalactosamine (GaINAc), such as those shown in FIG. 5. In one embodiment, the conjugate moiety is the tri-valent N-acetylgalactosamine (GaINAc) of FIG. 5A-1 or FIG. 5A-2, or a mixture of both. In one embodiment, the conjugate moiety is the tri-valent N-acetylgalactosamine (GaINAc) of FIG. 5B-1 or FIG. 5B-2, or a mixture of both. In one embodiment, the conjugate moiety is the tri-valent N-acetylgalactosamine (GaINAc) of FIG. 5C-1 or FIG. 5C-2, or a mixture of both. In one embodiment, the conjugate moiety is the tri-valent N-acetylgalactosamine (GaINAc) of FIG. 5D-1 or FIG. 5D-2, or a mixture of both.

In some embodiments, the conjugate is selected from the group consisting of

5′-GN2-C6ₒCₒaₒAₛAₛTₛTₛtₛtₛaₛ
CₛaₛtₛaₛCₛtₛCₛtₛgₛGₛTₛ,
5′-GN2-C6ₒCₒaₒAₛAₛtₛtₛtₛtₛaₛ
CₛaₛtₛaₛcₛtₛcₛtₛGₛGₛTₛ^{m}Gₛ,
5′-GN2-C6ₒcₒaₒ^{m}CₛTₛtₛtₛaₛtₛtₛ
aₛtₛaₛaₛCₛtₛTₛgₛaₛAₛtₛ^{m}CₛTₛ^{m}Cₛ,
and
5′-GN2-C6ₒcₒaₒ TₛTₛaₛcₛaₛtₛaₛ
cₛtₛcₛtₛgₛgₛtₛ^{m}CₛAₛAₛAₛ

wherein a capital letter represents a beta-D-oxy LNA nucleoside, a lower case letter represents a DNA nucleoside, wherein each LNA cytosine is 5-methyl cytosine, and wherein subscript s represents a phosphorothioate internucleoside linkage, and a subscript o represents a phosphodiester internucleoside linkage, and GN2-C6 is tri-valent N-acetylgalactosamine (GaINAc), such as those shown in FIG. 5, for example, such as the tri-valent N-acetylgalactosamine (GaINAc) of FIG. 5D-1 or FIG. 5D-2, or a mixture of both.

Chemical drawings representing some of the molecules are shown in FIGS. 1 to 4.

In some embodiments, the conjugate is the conjugate as shown in FIG. 1.

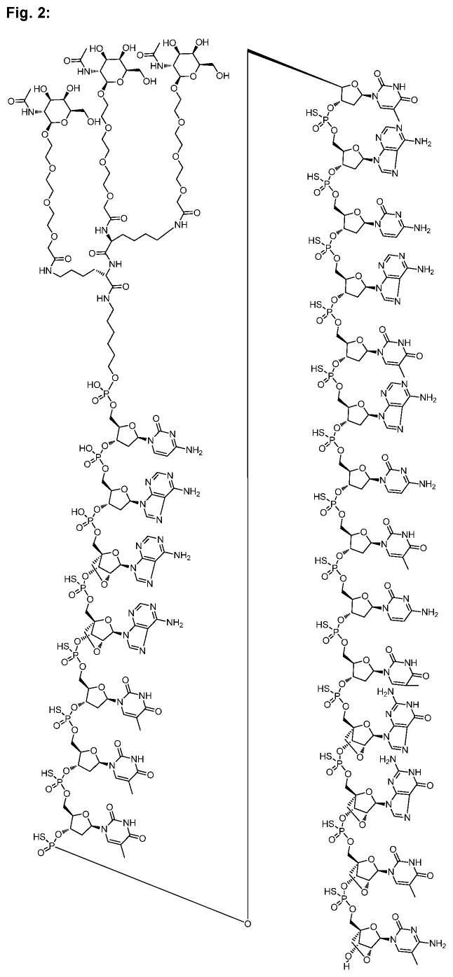

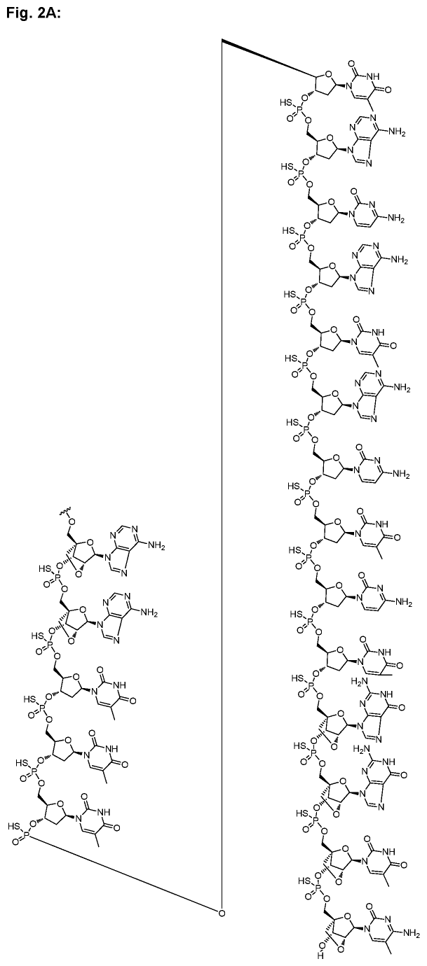

In some embodiments, the conjugate is the conjugate as shown in FIG. 2.

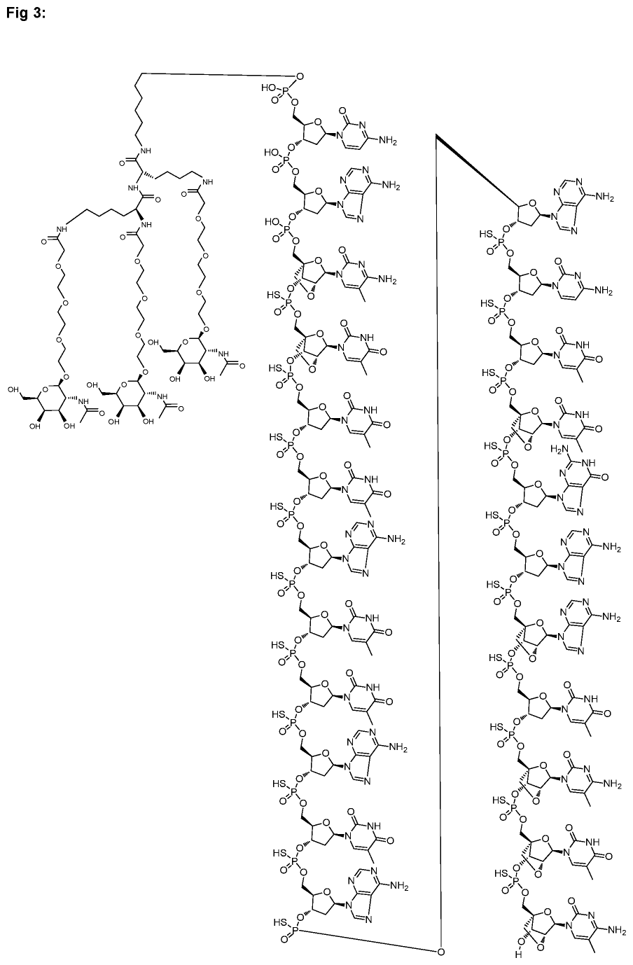

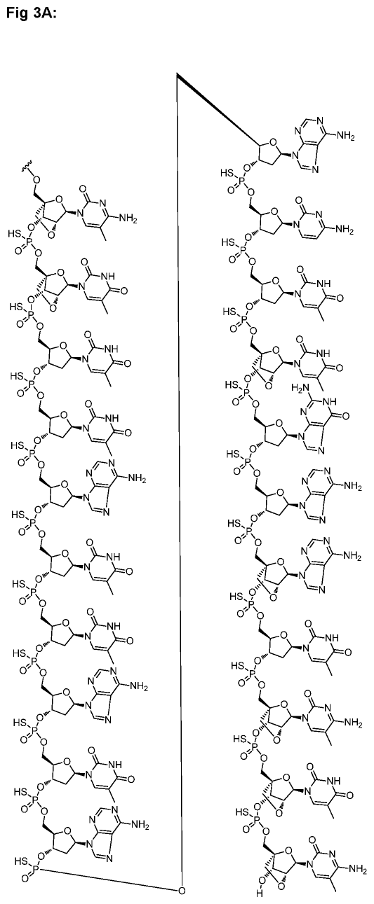

In some embodiments, the conjugate is the conjugate as shown in FIG. 3.

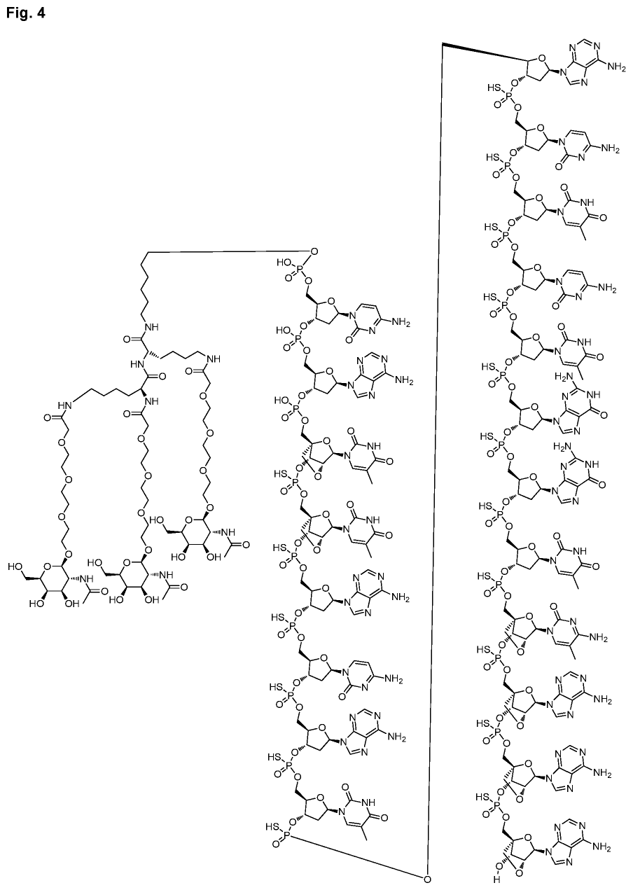

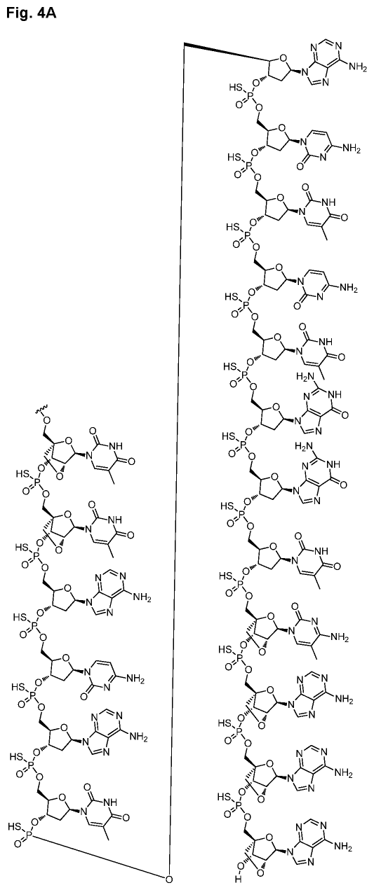

In some embodiments, the conjugate is the conjugate as shown in FIG. 4.

The compounds illustrated in FIGS. 1-4 are shown in the protonated form—the S atom on the phosphorothioate linkage is protonated—it will be understood that the presence of the proton will depend on the acidity of the environment of the molecule, and the presence of an alternative cation (e.g. when the oligonucleotide is in salt form). Protonated phosphorothioates exist in tautomeric forms.

### Pharmaceutically Acceptable Salts

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

In a further aspect the invention provides a pharmaceutically acceptable salt of the antisense oligonucleotide or a conjugate thereof, such as a pharmaceutically acceptable sodium salt, ammonium salt or potassium salt.

### Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phosphoramidite chemistry (see for example Caruthers et al, 1987, Methods in Enzymology vol. 154, pages 287-313).

In a further embodiment, the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

### Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments, the pharmaceutically acceptable diluent is sterile phosphate buffered saline. In some embodiments, the oligonucleotide is used in the pharmaceutically acceptable diluent at a concentration of 50-300 μM solution.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

In some embodiments, the antisense oligonucleotide of the invention or conjugate thereof, or pharmaceutically acceptable salt thereof is in a solid form, such as a powder, such as a lyophilized powder.

In some embodiments, the antisense oligonucleotide of the invention or conjugate thereof may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the antisense oligonucleotide of the invention or conjugate thereof is a prodrug. In particular, with respect to antisense oligonucleotide conjugates the conjugate moiety is cleaved off the oligonucleotide once the prodrug is delivered to the site of action, e.g. the target cell.

### Applications

The enhanced antisense oligonucleotides of the invention thereof may be utilized as research reagents for, for example, diagnostics, therapeutics and prophylaxis.

In research, such antisense oligonucleotides may be used to specifically modulate the synthesis of RTEL1 protein in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby prevent protein formation or by degrading or inhibiting a modulator of the gene or mRNA producing the protein.

If employing the antisense oligonucleotides of the invention in research or diagnostics the target nucleic acid may be a cDNA or a synthetic nucleic acid derived from DNA or RNA.

Also encompassed by the present invention is an in vivo or in vitro method for modulating RTEL1 expression in a target cell, which is expressing RTEL1, said method comprising administering an antisense oligonucleotide, a conjugate thereof or pharmaceutical composition of the invention in an effective amount to said cell.

In some embodiments, the target cell, is a mammalian cell in particular a human cell. The target cell may be an in vitro cell culture or an in vivo cell forming part of a tissue in a mammal. In preferred embodiments, the target cell is present in in the liver. The target cell may be a hepatocyte.

One aspect of the present invention is related the antisense oligonucleotides, conjugate thereof or pharmaceutical compositions of the invention for use as a medicament.

In an aspect of the invention the antisense oligonucleotides, conjugate thereof or pharmaceutical composition of the invention is capable of reducing the cccDNA level in the infected cells and therefore inhibiting HBV infection. In particular, the antisense oligonucleotide or conjugate thereof is capable of affecting one or more of the following parameters i) reducing cccDNA and/or ii) reducing pgRNA and/or iii) reducing HBV DNA and/or iv) reducing HBV viral antigens in an infected cell.

For example, the antisense oligonucleotide or conjugate thereof that inhibits HBV infection may reduce i) the cccDNA levels in an infected cell by at least 40% such as 50%, 60%, 70%, 80%, or 90% reduction compared to controls; or ii) the level of pgRNA by at least 40% such as 50%, 60%, 70%, 80%, or 90% reduction compared to controls. The controls may be untreated cells or animals, or cells or animals treated with an appropriate control.

Inhibition of HBV infection may be measured in vitro using HBV infected primary human hepatocytes or in vivo using humanized hepatocytes PXB mouse model (available at PhoenixBio, see also Kakuni et al 2014 Int. J. Mol. Sci. 15:58-74). Inhibition of secretion of HBsAg and/or HBeAg may be measured by ELISA, e.g. by using the CLIA ELISA Kit (Autobio Diagnostic) according to the manufacturers' instructions. Reduction of intracellular cccDNA or HBV mRNA and pgRNA may be measured by qPCR, e.g. as described in the Materials and Methods section. Further methods for evaluating whether a test compound inhibits HBV infection are measuring secretion of HBV DNA by qPCR e.g. as described in WO 2015/173208 or using Northern Blot; in-situ hybridization, or immuno-fluorescence.

Due to the reduction of RTEL1 levels the antisense oligonucleotide, conjugate thereof or pharmaceutical compositions of the present invention can be used to inhibit development of or in the treatment of HBV infection. In particular, the destabilization and reduction of the cccDNA, the antisense oligonucleotide, conjugate thereof or pharmaceutical compositions of the present invention more efficiently inhibits development of or treats a chronic HBV infection as compared to a compound that only reduces secretion of HBsAg.

Accordingly, one aspect of the present invention is related to use of the antisense oligonucleotide, conjugate thereof or pharmaceutical compositions of the invention to reduce cccDNA and/or pgRNA in an HBV infected individual.

A further aspect of the invention relates to the use of the antisense oligonucleotide, conjugate thereof or pharmaceutical compositions of the invention to inhibit development of or treat a chronic HBV infection.

A further aspect of the invention relates to the use of the antisense oligonucleotide, conjugate thereof or pharmaceutical compositions of the invention to reduce the infectiousness of a HBV infected person. In a particular aspect of the invention, the antisense oligonucleotide, conjugate thereof or pharmaceutical compositions of the invention inhibits development of a chronic HBV infection.

The subject to be treated with the antisense oligonucleotide, conjugate thereof or pharmaceutical compositions of the invention (or which prophylactically receives antisense oligonucleotides, conjugates thereof or pharmaceutical compositions of the present invention) is preferably a human, more preferably a human patient who is HBsAg positive and/or HBeAg positive, even more preferably a human patient that is HBsAg positive and HBeAg positive.

Accordingly, the present invention relates to a method of treating a HBV infection, wherein the method comprises administering an effective amount of the antisense oligonucleotide, conjugate thereof or pharmaceutical compositions of the invention. The present invention further relates to a method of preventing liver cirrhosis and hepatocellular carcinoma caused by a chronic HBV infection.

The invention also provides for the use of an antisense oligonucleotide, conjugate thereof or a pharmaceutical composition of the invention for the manufacture of a medicament, in particular a medicament for use in the treatment of HBV infection or chronic HBV infection or reduction of the infectiousness of a HBV infected person. In preferred embodiments, the medicament is manufactured in a dosage form for subcutaneous administration.

The invention also provides for the use of an antisense oligonucleotide, conjugate thereof, the pharmaceutical composition of the invention for the manufacture of a medicament wherein the medicament is in a dosage form for intravenous administration.

The antisense oligonucleotide, conjugate thereof or the pharmaceutical composition of the invention may be used in a combination therapy. For example, antisense oligonucleotide, conjugate thereof or the pharmaceutical composition of the invention may be combined with other anti-HBV agents such as interferon alpha-2b, interferon alpha-2a, and interferon alphacon−1 (pegylated and unpegylated), ribavirin, lamivudine (3TC), entecavir, tenofovir, telbivudine (LdT), adefovir, or other emerging anti-HBV agents such as a HBV RNA replication inhibitor, a HBsAg secretion inhibitor, a HBV capsid inhibitor, an antisense oligomer (e.g. as described in WO2012/145697, WO 2014/179629 and WO2017/216390), a siRNA (e.g. described in WO 2005/014806, WO 2012/024170, WO 2012/2055362, WO 2013/003520, WO 2013/159109, WO 2017/027350 and WO2017/015175), a HBV therapeutic vaccine, a HBV prophylactic vaccine, a HBV antibody therapy (monoclonal or polyclonal), or TLR 2, 3, 7, 8 or 9 agonists for the treatment and/or prophylaxis of HBV.

### Combination Therapy

In some embodiments, the enhanced antisense oligonucleotide, conjugate thereof or the pharmaceutical composition of the invention is for use in a combination treatment with another therapeutic agent. The therapeutic agent can for example be the standard of care for the diseases or disorders described above.

By way of example, the antisense oligonucleotide, conjugate thereof or the pharmaceutical composition may be used in combination with other actives, such as oligonucleotide-based antivirals—such as sequence specific oligonucleotide-based antivirals—acting either through antisense (including other LNA oligomers), siRNAs (such as ARC520), aptamers, morpholinos or any other antiviral, nucleotide sequence-dependent mode of action.

By way of further example, the antisense oligonucleotide, conjugate thereof or the pharmaceutical composition may be used in combination with other actives, such as immune stimulatory antiviral compounds, such as interferon (e.g. pegylated interferon alpha), TLR7 agonists (e.g. GS-9620), or therapeutic vaccines.

By way of further example, the antisense oligonucleotide, conjugate thereof or the pharmaceutical composition may be used in combination with other actives, such as small molecules, with antiviral activity. These other actives could be, for example, nucleoside/nucleotide inhibitors (eg entecavir or tenofovir disoproxil fumarate), encapsidation inhibitors, entry inhibitors (eg Myrcludex B).

In certain embodiments, the additional therapeutic agent may be an HBV agent, a Hepatitis C virus (HCV) agent, a chemotherapeutic agent, an antibiotic, an analgesic, a nonsteroidal anti-inflammatory (NSAID) agent, an antifungal agent, an antiparasitic agent, an anti-nausea agent, an anti-diarrheal agent, or an immunosuppressant agent.

In particular, related embodiments, the additional HBV agent may be interferon alpha-2b, interferon alpha-2a, and interferon alphacon−1 (pegylated and unpegylated), ribavirin; an HBV RNA replication inhibitor; a second antisense oligomer; an HBV therapeutic vaccine; an HBV prophylactic vaccine; lamivudine (3TC); entecavir (ETV); tenofovir disoproxil fumarate (TDF); telbivudine (LdT); adefovir; or an HBV antibody therapy (monoclonal or polyclonal).

In other particular related embodiments, the additional HCV agent may be interferon alpha-2b, interferon alpha-2a, and interferon alphacon−1 (pegylated and unpegylated); ribavirin; pegasys; an HCV RNA replication inhibitor (e.g., ViroPharma's VP50406 series); an HCV antisense agent; an HCV therapeutic vaccine; an HCV protease inhibitor; an HCV helicase inhibitor; or an HCV monoclonal or polyclonal antibody therapy.

### Administration

The enhanced antisense oligonucleotides, conjugates thereof or pharmaceutical compositions of the present invention may be administered topical (such as, to the skin, inhalation, ophthalmic or otic) or enteral (such as, orally or through the gastrointestinal tract) or parenteral (such as, intravenous, subcutaneous, or intra-muscular).

In a preferred embodiment, the antisense oligonucleotide, conjugates thereof or pharmaceutical compositions of the present invention are administered by a parenteral route including intravenous, intraarterial, subcutaneous, intraperitoneal or intramuscular administration. In one embodiment, the active antisense oligonucleotide or conjugate thereof is administered intravenously. In another embodiment, the active antisense oligonucleotide or conjugate thereof is administered subcutaneously.

In some embodiments, the antisense oligonucleotide, conjugate thereof or pharmaceutical composition of the invention is administered at a dose of 0.1-15 mg/kg, such as from 0.2-10 mg/kg, such as from 0.25-5 mg/kg. The administration can be once a week, every second week, every third week or even once a month.

The invention also provides for the use of the antisense oligonucleotide, or conjugate thereof of the invention as described for the manufacture of a medicament wherein the medicament is in a dosage form for subcutaneous administration.

### Embodiments of the Invention

The following embodiments of the present invention may be used in combination with any other embodiments described herein. The definitions and explanations provided herein above, in particular in the sections “SUMMARY OF INVENTION”, “DEFINITIONS” and DETAILED DESCRIPTION OF THE INVENTION” apply mutatis mutandis to the following.
1. An antisense oligonucleotide which comprises a contiguous nucleotide sequence with at least 90% complementarity, such as 100% complementarity to a RTEL1 nucleic acid, wherein the antisense oligonucleotide is capable of inhibiting the expression of RTEL1, such as human RTEL1, in a cell.
2. The antisense oligonucleotide of embodiment 1, wherein the contiguous nucleotide sequence is fully complementary to a region from nucleotides 11753-11774 of the human RTEL1 pre-mRNA as shown in in SEQ ID NO: 1, such as to a region from nucleotides 11757-11774, from nucleotides 11756-11774, or from nucleotides 11753-11770 of SEQ ID NO: 1.
3. The antisense oligonucleotide of embodiments 1 and 2, wherein the contiguous nucleotide sequence is fully complementary to SEQ ID NO: 7, 8, 9 and/or 10.
4. The antisense oligonucleotide of embodiment 1, wherein the contiguous nucleotide sequence is fully complementary to a region from nucleotides 8681-8701 of the human RTEL1 pre-mRNA as shown in in SEQ ID NO: 1.
5. The antisense oligonucleotide of embodiments 1 and 4, wherein the contiguous nucleotide sequence is fully complementary to SEQ ID NO: 11.
6. The antisense oligonucleotide of any one of embodiments 1 to 5, wherein the antisense oligonucleotide is 12-30 nucleotides in length, such as 12 to 22 nucleotides in length, such as 16 to 20 nucleotides in length.
7. The antisense oligonucleotide of any one of embodiments 1 to 6, wherein the contiguous nucleotide sequence is a contiguous sequence of at least 12 nucleotides, such as of 14, 15, 16, 17, 18, 19, or 20 nucleotides.
8. The antisense oligonucleotide of embodiment 7, wherein the contiguous nucleotide sequence is a contiguous sequence of 18 or 19 nucleotides.
9. The antisense oligonucleotide of any one of embodiments 1 to 8, wherein the contiguous nucleotide sequence is 100% identical to a sequence selected from the group consisting of SEQ ID NOs: 3, 4, 5 and 6, or at least 15 contiguous nucleotides thereof.
10. The antisense oligonucleotide of any one of embodiments 1 to 9, comprising one or more modified nucleosides in the contiguous nucleotide sequence.
11. The antisense oligonucleotide of embodiment 10, wherein the one or more modified nucleosides in the contiguous nucleotide sequence are 2′ sugar modified nucleosides.
12. The antisense oligonucleotide of embodiment 11, wherein the one or more 2′ sugar modified nucleosides are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.
13. The antisense oligonucleotide of any one of embodiments 10 to 12, wherein the one or more modified nucleosides are LNA nucleosides, such as oxy-LNA with the following 2′-4′ bridge —O—CH₂—.
14. The antisense oligonucleotide of embodiment 13, wherein the one or more modified nucleosides are beta-D-oxy-LNA.
15. The antisense oligonucleotide of any one of embodiments 1 to 14, wherein at least one internucleoside linkage in the contiguous nucleotide sequence is a phosphorothioate internucleoside linkage.
16. The antisense oligonucleotide of any one of embodiments 1 to 15, wherein at least one internucleoside linkage in the contiguous nucleotide sequence is a phosphorodithioate internucleoside linkage.
17. The antisense oligonucleotide of any one of embodiments 1 to 16, wherein at least one internucleoside linkage in the contiguous nucleotide sequence is a phosphodiester internucleoside linkage.
18. The antisense oligonucleotide of embodiment 17, wherein all the internucleoside linkages within the contiguous nucleotide sequence are phosphorothioate internucleoside linkages.
19. The antisense oligonucleotide of any one of embodiments 1 to 18, wherein the antisense oligonucleotide is an antisense oligonucleotide which is capable of recruiting RNase H, such as RNase H1.
20. The antisense oligonucleotide of embodiment 19, wherein the antisense oligonucleotide, or contiguous nucleotide sequence thereof, consists of or comprises a gapmer of formula 5′-F-G-F′-3′.
21. The antisense oligonucleotide according to embodiment 20, wherein region G has a length of 6 to 16 DNA nucleosides, such as 11 to 16 DNA nucleosides.
22. The antisense oligonucleotide according to any one of embodiments 19 to 21, wherein region F and F′ each comprise at least one LNA nucleoside, for example wherein region F and F′ each comprise at least one LNA nucleoside.
23. The antisense oligonucleotide according to any one of embodiments 19 to 22, wherein region F has a length of 1 to 8 DNA nucleosides, such as of 2 to 4 DNA nucleosides.
24. The antisense oligonucleotide according to any one of embodiments 19 to 23, wherein region F′ has a length of 1 to 8 DNA nucleosides, such as of 2 to 6 DNA nucleosides.
25. The antisense oligonucleotide according to any one of embodiments 19 to 24, wherein the antisense oligonucleotide, or contiguous nucleotide sequence thereof, consists of or comprises a gapmer of formula F₂₄-G₁₁₁₆-F′₂-6.
26. The antisense oligonucleotide according to any one of embodiments 19 to 25, wherein the gapmer comprises at least one alternating flank.
27. The antisense oligonucleotide according to any one of embodiments 19 to 25, wherein the gapmer comprises two uniform flanks.
28. The antisense oligonucleotide according to any one of embodiments 1 to 27, wherein the antisense oligonucleotide is selected from the group of antisense oligonucleotides consisting of

(SEQ ID NO: 3, Compound ID No 3_1),
AATTttacatactctgGT
(SEQ ID NO: 4, Compound ID No 4_1),
AAttttacatactctGGTC
(SEQ ID NO: 5, Compound ID No 5_1),
TTacatactctggtCAAA
and
(SEQ ID NO: 6, Compound ID No 6_1),
CTttattataactTgaAtCTC

wherein capital letters are beta-D-oxy LNA nucleosides, lowercase letters are DNA nucleosides, all LNA C are 5-methyl cytosine, all internucleoside linkages are phosphorothioate internucleoside linkages.
29. A conjugate comprising the antisense oligonucleotide according to any one of embodiments 1 to 28, and at least one conjugate moiety covalently attached to said antisense oligonucleotide.
30. The conjugate of embodiment 29, wherein the conjugate moiety comprises at least one asialoglycoprotein receptor targeting moiety selected from the group consisting of galactose, galactosamine, N-formyl-galactosamine, N-acetylgalactosamine, N-propionyl-galactosamine, N-n-butanoyl-galactosamine and N-isobutanoylgalactosamine.
31. The conjugate of embodiment 30, wherein the asialoglycoprotein receptor targeting moiety is N-acetylgalactosamine (GaINAc).
32. The compound of embodiment 30 or 31, wherein the conjugate moiety is mono-valent, di-valent, tri-valent or tetra-valent with respect to asialoglycoprotein receptor targeting moieties.
33. The compound of embodiment 32, wherein the conjugate moiety consists of two to four terminal GaINAc moieties and a spacer linking each GaINAc moiety to a brancher molecule that can be conjugated to the antisense compound.
34. The conjugate of embodiment 33, wherein the spacer is a PEG spacer.
35. The conjugate of any one of embodiments 30 to 34, wherein the conjugate moiety is a tri-valent N-acetylgalactosamine (GaINAc) moiety.
36. The compound of any one of embodiments 30 to 35, wherein the conjugate moiety is selected from one of the trivalent GaINAc moieties in FIG. 5.
37. The conjugate of embodiment 36, wherein the conjugate moiety is the trivalent GaINAc moiety in FIG. 5, such as the trivalent GaINAc moiety of FIG. 5D-1 or FIG. 5D-2, or a mixture of both.
38. The conjugate of any one of embodiments 29 to 37, comprising a linker, which is positioned between the antisense oligonucleotide and the conjugate moiety.
39. The conjugate of embodiment 38, wherein the physiologically labile linker comprises or consists of 2 to 5 consecutive phosphodiester linked nucleosides, such as 2 consecutive phosphodiester linked nucleosides.
40. The conjugate according to any one of embodiments 29 to 39, wherein the conjugate is selected from the group consisting of

5′-GN2-C6ₒcₒaₒAₛAₛTₛTₛtₛtₛaₛ
cₛaₛtₛaₛcₛtₛcₛtₛgₛGₛT,
5′-GN2-C6ₒcₒaₒAₛAₛtₛtₛtₛtₛaₛ
cₛaₛtₛaₛcₛtₛcₛtₛGₛGₛTₛ^{m}C,
5′-GN2-C6ₒcₒaₒ^{m}CₛTₛtₛtₛaₛtₛtₛ
aₛtₛaₛaₛcₛtₛTₛgₛaₛAₛtₛ^{m}CₛTₛ^{m}C,
and
5′-GN2-C6ₒcₒaₒ TₛTₛaₛcₛaₛtₛaₛ
cₛtₛcₛtₛgₛgₛtₛ^{m}CₛAₛAₛA

wherein a capital letter represents a beta-D-oxy LNA nucleoside, a lower case letter represents a DNA nucleoside, wherein each LNA cytosine is 5-methyl cytosine, and ^{m}c is 5-methyl cytosine DNA, and wherein subscript s represents a phosphorothioate internucleoside linkage, and a subscript o represents a phosphodiester internucleoside linkage, GN2-C6 is a residue of formula:

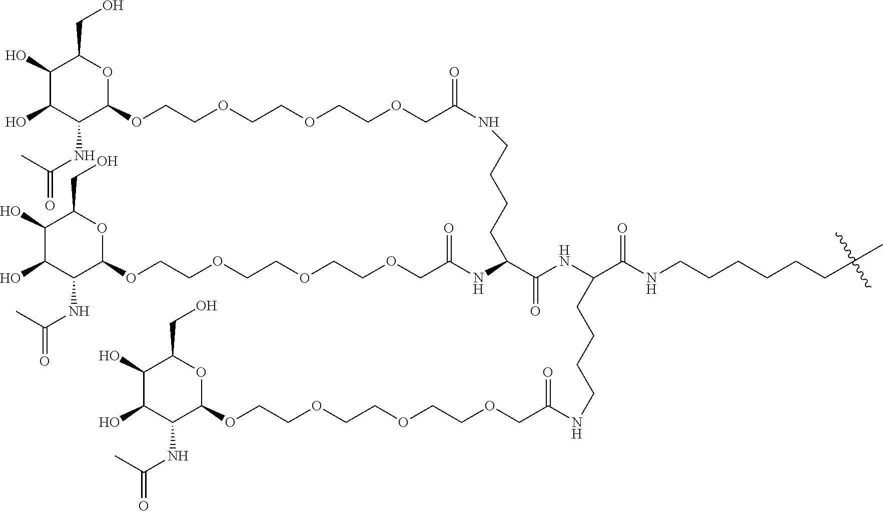 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

wherein the residue GN2-C6 is attached via a phosphodiester linkage at the 5′ end of the oligonucleotide, and/or wherein GN2-C6 is a tri-valent N-acetylgalactosamine (GaINAc) of FIG. 5D1 or FIG. 5D2, or a mixture of both, more preferably wherein GN2-C6 is a mixture of the tri-valent N-acetylgalactosamine (GaINAc) residues depicted in FIG. 5D1 or FIG. 5D2
41. A conjugate as shown in FIG. 1.
42. A conjugate as shown in FIG. 2.
43. A conjugate as shown in FIG. 3.
44. A conjugate as shown in FIG. 4.
45. A pharmaceutically acceptable salt of the oligonucleotide of any one of embodiments 1 to 28, or the conjugate according to any one of embodiments 29 to 44.
46. A pharmaceutical composition comprising the antisense oligonucleotide of any one of embodiments 1 to 28, the conjugate of any one of embodiments 29 to 44, or the pharmaceutically acceptable salt of embodiment 45, and a pharmaceutically acceptable diluent, solvent, carrier, salt and/or adjuvant.
47. An in vivo or in vitro method for modulating RTEL1 expression in a target cell which is expressing RTEL1, said method comprising administering the antisense oligonucleotide of any one of embodiments 1 to 28, the conjugate of any one of embodiments 29 to 44, the pharmaceutically acceptable salt of embodiment 45, or the pharmaceutical composition of embodiment 46 in an effective amount to said cell.
48. A method for treating or preventing a disease comprising administering a therapeutically or prophylactically effective amount of the antisense oligonucleotide of any one of embodiments 1 to 28, the conjugate of any one of embodiments 29 to 44, the pharmaceutically acceptable salt of embodiment 45, or the pharmaceutical composition of embodiment 46 to a subject suffering from or susceptible to the disease, wherein the disease is hepatitis B virus (HBV) infection, such as of chronic HBV infection.
49. The antisense oligonucleotide of any one of embodiments 1 to 28, the conjugate of any one of embodiments 29 to 44, the pharmaceutically acceptable salt of embodiment 45, or the pharmaceutical composition of embodiment 46 for use in medicine.
50. The antisense oligonucleotide of any one of embodiments 1 to 28, the conjugate of any one of embodiments 29 to 44, the pharmaceutically acceptable salt of embodiment 45, or the pharmaceutical composition of embodiment 46 for use in the treatment or prevention of hepatitis B virus (HBV) infection, such as of chronic HBV infection.
51. Use of the antisense oligonucleotide of any one of embodiments 1 to 28, the conjugate of any one of embodiments 29 to 44, the pharmaceutically acceptable salt of embodiment 45, or the pharmaceutical composition of embodiment 46, for the preparation of a medicament for treatment or prevention of hepatitis B virus (HBV) infection, such as of chronic HBV infection.

### EXAMPLES

### Materials and Methods

### Oligonucleotide Synthesis

Oligonucleotide synthesis is generally known in the art. Below is a protocol, which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphodiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phosphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

The crude compounds are purified by preparative RP-HPLC on a Phenomenex Jupiter® C18 10μ 150×10 mm column. 0.1 M ammonium acetate pH 8 and acetonitrile is used as buffers at a flow rate of 5 mL/min. The collected fractions are lyophilized to give the purified compound typically as a white solid.

### ABBREVIATIONS

DCI: 4,5-Dicyanoimidazole
DCM: Dichloromethane
DMF: Dimethylformamide
DMT: 4,4′-Dimethoxytrityl
THF: Tetrahydrofurane
Bz: Benzoyl
Ibu: Isobutyryl
RP-HPLC: Reverse phase high performance liquid chromatography

### Primary Human Hepatocytes (PXB-PHH)

Fresh primary human hepatocytes (PXB-PHH) harvested from humanized mice (uPA/SCID mice)—herein called PHH—were obtained from PhoenixBio Co., Ltd (Japan) in 96-well format and cultured in modified hepatocyte clonal growth medium (dHCGM). dHCGM is a DMEM medium containing 100 U/ml Penicillin, 100 μg/ml Streptomycin, 20 mM Hepes, 44 mM NaHCO₃, 15 μg/ml L-proline, 0.25 μg/ml Insulin, 50 nM Dexamethasone, 5 ng/ml EGF, 0.1 mM Asc-2P, 2% DMSO and 10% FBS (Ishida et al., 2015).

Cells were cultured at 37° C., in a humidified atmosphere with 5% CO₂. Culture medium was replaced every 2 days until harvest, except over the weekend.

### HBV Infection and Oligonucleotide Treatment

PHH were incubated with HBV (purified from chronic hepatitis B (CHB) individuals) at multiplicity of infection (MOI) of 40 together with 4% PEG for 24 hr. The virus inoculum was removed the following day and cells were washed 3 times with PBS before addition of fresh medium.

To allow for cccDNA establishment, compound treatment in PHH was started at day 3 post HBV infection. The cells were dosed in a 1:10 dilution step dose response manner starting at 10 μM. On Day 3, Day 5 and Day 7 post HBV infection, the cells were dosed with oligonucleotide compounds in a final volume of 100 μl/well of dHCGM Medium. 10 nM Entecavir (ETV) treatment was started at Day 5 post infection, ensuring real cccDNA measurement by qPCR, and medium including 10 nM ETV was changed every two days (except over the weekend) until cells were harvested at Day 16 post HBV infection. The experiments were performed in biological triplicates.

### Real-Time PCR for Intracellular RTEL1 RNA

Total mRNA was extracted from the cells using a Qiagen BioRobot Universal System and the RNeasy 96 well Extraction Plates (RNeasy 96 BioRobot 8000 Kit (12)/Cat No. II D: 967152) according to the manufacturer's protocol. The mRNA expression levels were analyzed using Real-time PCR on the ABI QuantStudio™ 12k Flex. Beta-actin (ACT B) was quantified by qPCR using TaqMan Fast Advanced Master Mix (Life Technologies, cat no. 4444558) in technical duplicates. qPCR for RTEL1 gene was performed with the Fast SYBR™ Green Master Mix (Life Technologies, Cat. No 4385612). Results were normalized over the human ACT B endogenous control. The mRNA expression was analyzed using the comparative cycle threshold 2-ΔΔCt method normalized to the reference gene ACT B and to non-treated cells. Primers used for ACTB RNA and RTEL1 RNA quantification are listed in Table 7:

TABLE 7
ACT B and RTEL1 RNA qPCR primers

| | | | Seq |
|---|---|---|---|
| | | | ID |
| Parameter | Direction | Primer Sequence | No |
| RTEL1 | Fwd | 5′- CCATCCTGGAC | 15 |
| | | ATTGAGGACT-3′ | |
| | Rev | 5′- CAGGTTCCGGG | 16 |
| | | ACAGGTAGTA-3′ | |

Housekeeping gene primers ACT B (VIC): Hs01060665_g1 (Thermo Fisher Scientific)

HBV cccDNA Quantification

DNA was extracted from HBV infected Primary Human Hepatocytes using an SDS Lysis Buffer (50 mM Tris pH8, 5 mM EDTA, 1% SDS). After lysing cells in 80 μl SDS lysis buffer, samples were frozen at −80° C. for minimum 2 hours. Samples were thawed at 37° C. and 1 μl of Proteinase K (cat no. AM25448 @ Ambion biosciences, 20 mg/mL Stock) was added to each well of the 96-well plate and samples were incubated at 56° C. for 30 minutes. After incubation, 3 volumes of ChIP DNA binding buffer from ZYMO Research Genomic DNA Clean & Concentrator kit (ZymoResearch, cat no. D4067) were added and DNA was purified following the manufacturer's protocol. DNA was eluted in 20 μl DNA Elution buffer and qPCR was performed using 2 μl DNA.

The cccDNA expression levels were quantified in technical duplicates using the comparative cycle threshold 2-ΔΔCt method. Quantitative real-time polymerase chain reaction measurements were performed on the QuantStudio 12K Flex PCR System (Applied Biosystems). Normalization was done to mitochondrial DNA (mitoDNA) and to non-treated cells as endogenous control using the Fast SYBR™ Green Master Mix (Life Technologies, Cat. No 4385612). Cycler settings were adjusted to incubation at 95° C. for 5 min, then 45 cycles of 95° C. for 1 sec and 60° C. for 35 sec. Primers used are listed Table 8 below (all probes in the chart are SYBR Green):

TABLE 8
cccDNA qPCR primers.

| | | | Seq |
|---|---|---|---|
| | | | ID |
| Parameter | Direction | Primer Sequence | No |
| cccDNA | Fwd | 5′-CGTCTGTGCCT | 17 |
| | | TCTCATCTGC-3′ | |
| | Rev | 5′-GCACAGCTTGG | 18 |
| | | AGGCTTGAA-3′ | |
| mitochondrial | Fwd | 5′-CCGTCTGAACT | 19 |
| DNA | | ATCCTGCCC-3′ | |
| | Rev | 5′-GCCGTAGTCGG | 20 |
| | | TGTACTCGT-3′ | |

### Example 1: Effect of Antisense Oligonucleotides Targeting RTEL1 on RTEL1 RNA and on cccDNA in HBV Infected PHH Cells

The effects of RTEL1 knock-down on RTEL1 RNA and on cccDNA were tested using the oligonucleotide compounds from Table 6. PHH were cultured as described in the Materials and Methods section. HBV infected PHH cells were treated with the compounds from Table 6 as described above. Following the 16 days-treatment, RTEL1 mRNA and cccDNA were measured by qPCR as described above. The results are shown in Table 9 as % of the average no drug control (NDC) samples (i.e. the lower the value the larger the inhibition/reduction).

TABLE 9
Effect on cccDNA following knockdown
of RTEL1 with LNA ASO

| | | | cccDNA | | RTEL1 | |
|---|---|---|---|---|---|---|
| CMP | SEQ | | % of | | % of | |
| ID | ID | | control | cccDNA | control | RTEL1 |
| NO | NO | Compound | (Mean) | SD | (Mean) | SD |
| 3_1 | 3 | AATTttaca | 28 | 13 | 19 | 3 |
| | | tactctgGT | | | | |
| 4_1 | 4 | AAttttaca | 3 | 1 | 24 | 2 |
| | | tactctGGT | | | | |
| | | C | | | | |
| 5_1 | 5 | TTacatact | 21 | 6 | 35 | 11 |
| | | ctggtCAAA | | | | |
| 6_1 | 6 | CTttattat | 39 | 5 | 21 | 8 |
| | | aactTgaAt | | | | |
| | | CTC | | | | |

For Compounds: Capital letters represent LNA nucleosides (beta-D-oxy LNA nucleosides were used), all LNA cytosines are 5-methyl cytosine, lower case letters represent DNA nucleosides. All internucleoside linkages are phosphorothioate internucleoside linkages.

### Example 2: Testing In Vitro Efficacy of Antisense Oligonucleotides Targeting RTEL1 mRNA in Human MDA-MB-231 Cell Line at Different Concentrations for a Concentration Response Curve

Human MDA-MB-231 cell lines were purchased from ATCC and maintained as recommended by the supplier in a humidified incubator at 37° C. with 5% CO₂. For assays, 3500 cells/well of were seeded in a 96 multi well plate in culture media. Cells were incubated for 24 hours before addition of oligonucleotides dissolved in PBS. Highest screening concentration of oligonucleotides: 50 μM and subsequent 1:1 dilutions in 8 steps. 3 days after addition of oligonucleotides, the cells were harvested. RNA was extracted using the PureLink™ Pro 96 RNA Purification kit (Thermo Fisher Scientific) according to the manufacturer's instructions and eluted in 50 μl water. The RNA was subsequently diluted 10 times with DNase/RNase free Water (Gibco) and heated to 90° C. for one minute.

For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: RTEL1_Hs00249668_m1 [FAM-MGB] and endogenous control GUSB_Hs99999908_m1 [VIC-MGB]. All primer sets were purchased from Thermo Fisher Scientific. IC50 determination was performed in GraphPad Prism7.04 from biological replicates n=2. The relative RTEL1 mRNA level at treatment with 50 μM oligonucleotide is shown in Table 10 as percent of control (PBS treated samples).

TABLE 10
IC₅₀ values and mRNA levels at Max K_{D}

| | | | | | | mRNA | |
|---|---|---|---|---|---|---|---|
| | | | | IC₅₀ | | level | |
| | | | | in | | at Max | |
| SEQ | | CMP | | MDA- | | KD in | |
| ID | | ID | | MB- | | MDA- | |
| NO | Motif | NO | Compound | 231 | SD | MB-231 | SD |
| 3 | aattttaca | 3_1 | AATTttaca | 0.6 | 0.1 | 38 | 1 |
| | tactctggt | | tactctgGT | | | | |
| 4 | aattttaca | 4_1 | AAttttaca | 0.3 | 0.1 | 14 | 1 |
| | tactctggt | | tactctGGT | | | | |
| | c | | C | | | | |
| 5 | ttacatactc | 5_1 | TTacatact | 0.9 | 0.3 | 9 | 4 |
| | tggtcaaa | | ctggtCAAA | | | | |
| 6 | ctttattat | 6_1 | CTttattat | 0.4 | 0.1 | 18 | 3 |
| | aacttgaat | | aactTgaAt | | | | |
| | ctc | | CTC | | | | |

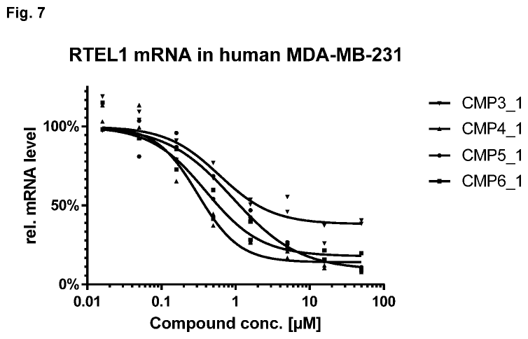

The compounds show very good efficacy and potency towards knockdown of human RTEL1 mRNA as the concentration response curves in human cell line MDA-MB-231 display, provided in FIG. 7.

### Example 3: Direct Comparison of In Vivo Efficacy of Compounds of the Present Invention and Prior Art Compounds

In this experiment, the compounds of the present invention (CMP ID NOs: 3_1, 4_1, 5_1 and 6_1, see e.g. Table 6) were compared with compound disclosed in WO 2020/011902 A1. The tested prior art compounds are shown in Table 11 (CMP ID NOs: 7_1 to 23_1). Specifically, the effect on the RTEL1 mRNA level in human MDA-MB-231 cells was tested.

Human MDA-MB-231 cell line was purchased from ATCC and maintained as recommended by the supplier in a humidified incubator at 37° C. with 5% CO₂. For assays, 3500 cells/well of were seeded in a 96 multi well plate in culture media. Cells were incubated for 24 hours before addition of oligonucleotides dissolved in PBS. Highest screening concentration of oligonucleotides: 50 μM and subsequent 1:1 dilutions in 8 steps. 3 days after addition of oligonucleotides, the cells were harvested. RNA was extracted using the PureLink™ Pro 96 RNA Purification kit (Thermo Fisher Scientific) according to the manufacturer's instructions and eluted in 50 μl water. The RNA was subsequently diluted 10 times with DNase/RNase free Water (Gibco) and heated to 90° C. for one minute.

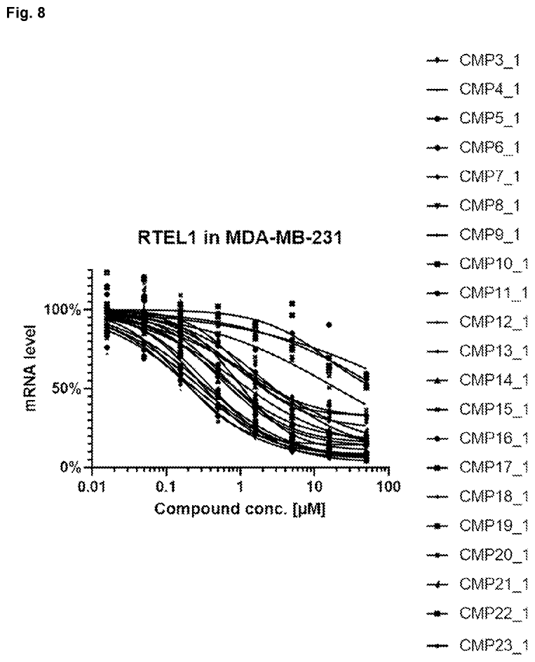

For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: RTEL1_Hs00249668_m1 [FAM-MGB] and endogenous control GUSB_Hs99999908_m1 [VIC-MGB]. All primer sets were purchased from Thermo Fisher Scientific. IC50 determination was performed in GraphPad Prism7.04 from biological replicates n=2. The relative RTEL1 mRNA level at treatment with 50 μM oligonucleotide is shown in Table 11 as percent of control (PBS treated samples). The results are also shown in FIG. 8.

TABLE 11
IC50 values and mRNA levels at Max KD

| | | | | mRNA |
|---|---|---|---|---|
| | | | | level |
| | | | | at |
| | | | | Max |
| | | | IC₅₀ | KD |
| | | | In | in |
| | SEQ | | MDA- | MDA- |
| CMP | ID | | MB- | MB- |
| ID | NO | Compound | 231 | 231 |
| 3_1 | 3 | AATTttacatactctgGT | 0.7 | 32 |
| 4_1 | 4 | AAttttacatactctGGTC | 0.2 | 6 |
| 5_1 | 5 | TTacatactctggtCAAA | 0.9 | 9 |
| 6_1 | 6 | CTttattataactTgaAtCTC | 0.2 | 7 |
| 7_1 | 21 | GAattttacatactctGGT | 0.6 | 15 |
| 8_1 | 22 | GAAttttacatactctgGTC | 0.3 | 8 |
| 9_1 | 23 | CAAaaaacagtaggTCC | 2.0 | 18 |
| 10_1 | 24 | Gagggaggtggag^{m}cgTT | — | 55 |
| 11_1 | 25 | AGCTttattataacttgaAT | — | 55 |
| 12_1 | 26 | TTttacatactctggtCAAA | 0.8 | 17 |
| 13_1 | 27 | TTTtacatactctggTCA | 0.4 | 4 |
| 14_1 | 28 | GAgaattttacatactcTGG | 0.6 | 16 |
| 15_1 | 29 | GCatccaacaagtaattGT | — | 35 |
| 16_1 | 30 | GgtgggtggatgTTTC | 0.8 | 32 |
| 17_1 | 31 | GgtggtgtggagaAGC | — | 52 |
| 18_1 | 32 | GGCatccaacaagtaaTT | 2.1 | 22 |
| 19_1 | 33 | GGaataaaacagtaGGTC | 0.5 | 18 |
| 20_1 | 34 | GCcattttcactgtCAA | 0.3 | 7 |
| 21_1 | 35 | GTGcagaagtcagaacaAA | 0.9 | 26 |
| 22_1 | 36 | CAAAatgcccttacagtGA | 0.3 | 6 |
| 23_1 | 37 | GGAataaaacagtaGGT | 0.2 | 13 |

### Example 4: Antisense Oligonucleotide Caspase Screen in HepG2 and 3T3 Cells for 24 Hours at 100 nM Concentration

In this experiment, the toxicity of the compounds of the present invention (CMP ID NOs: 3_1, 4_1, 5_1 and 6_1, see e.g. Table 6) was assessed by a Caspase screen in HepG2 cells. The prior art compounds tested in Example 3 were included in this study. Further, shorter metabolites of the full-lengths CMP ID NOs: 3_1 and 5_1 were analysed (for more details, see Example 6).

HepG2 or 3T3 cells were cultivated at app. 70% confluence in MEM medium with GlutaMax (Gibco #41090; HepG2) or DMEM with Glutamax (Gibco #31966), completed with 5 mM HEPES (3T3), supplemented with 10% heat inactivated fetal calf serum. Cells were detached with 0.25% Trypsin-EDTA solution (Gibco #25200056) and seeded into black, clear 96-well plates (Corning #3904, NY, USA) at a density of 1×10⁴ cells/well (HepG2) or 0.25×10⁴ cells/well (3T3). 24 h post-seeding cells were transiently transfected with Lipofectamine 2000 (Life Technologies #11668019) using 100 nM oligonucleotides dissolved in Opti-MEM (Gibco #31985). Caspase-3/7 activity was determined using the Caspase-Glo® 3/7 Assay (Promega Corporation, Madison WI, USA). Reconstituted Caspase-Glo® 3/7 reagent was added to the cells 24 hours post-transfection, incubated for 60 min, cell lysates were transferred into opaque 96-well plates (Corning #3600, NY, USA) before luminescence was determined on an Enspire multi-mode plate reader (Perkin Elmer) according to the manufacturer's instructions.

The results are shown in Table 12.

TABLE 12
Results of caspase scoring

| | | | Caspase | Caspase |
|---|---|---|---|---|
| | SEQ | | (% AW) | (% AW) |
| | ID | | in | in |
| CMP-ID | NO | Compound | HepG2 | 3T3 |
| 3_1 | 3 | AATTttacatactctgGT | −4 | 20 |
| 3_1-1 | 38 | AATTttacatactctgG | −5 | 11 |
| 3_1-2 | 39 | AATTttacatactctg | −6 | 3 |
| 4_1 | 4 | AAttttacatactctGGTC | 32 | 23 |
| 5_1 | 5 | TTacatactctggtCAAA | 8 | 29 |
| 5_1-1 | 40 | TTacatactctggtCAA | 8 | 30 |
| 5_1-2 | 41 | TTacatactctggtCA | 0 | 13 |
| 6_1 | 6 | CTttattataactTgaAtCTC | 6 | 22 |
| 7_1 | 21 | GAattttacatactctGGT | 2 | 26 |
| 8_1 | 22 | GAAttttacatactctgGTC | 11 | 35 |
| 9_1 | 23 | CAAaaaacagtaggTCC | 2 | 26 |
| 10_1 | 24 | Gagggaggtggag^{m}cgTT | 11 | −7 |
| 11_1 | 25 | AGCTttattataacttgaAT | 5 | 47 |
| 12_1 | 26 | TTttacatactctggtCAAA | 23 | 72 |
| 13_1 | 27 | TTTtacatactctggTCA | 33 | 47 |
| 14_1 | 28 | GAgaattttacatactcTGG | 13 | 44 |
| 15_1 | 29 | GCatccaacaagtaattGT | 53 | 102 |
| 16_1 | 30 | GgtgggtggatgTTTC | 114 | 12 |
| 17_1 | 31 | GgtggtgtggagaAGC | 110 | 80 |
| 18_1 | 32 | GGCatccaacaagtaaTT | 94 | 144 |
| 19_1 | 33 | GGaataaaacagtaGGTC | 44 | 73 |
| 20_1 | 34 | GCcattttcactgtCAA | 59 | 23 |
| 21_1 | 35 | GTGcagaagtcagaacaAA | 20 | 69 |
| 22_1 | 36 | CAAAatgcccttacagtGA | 143 | 44 |
| 23_1 | 37 | GGAataaaacagtaGGT | 70 | 51 |

Compounds with a value of more than 60% AW were considered as toxic (T). Compounds with a value of 40-60% AW were considered as medium toxic (MT). Compounds with a value of 20-40% AW were considered as mild toxic (M). Compounds with a value of less than 20% AW were considered as non-toxic (NT) As it can be derived from Table 12, the compounds with CMP ID NO: 11_1 (MT), CMP ID NO: 12_1 (T), CMP ID NO: 13_1 (MT), CMP ID NO: 141 (MT), CMP ID NO: 15_1(T), CMP ID NO: 16_1(T), CMP ID NO: 171 (T), CMP ID NO: 18_1(T), CMP ID NO: 19_1(T), CMP ID NO: 201 (MT), CMP ID NO: 21_1, CMP ID NO: 22_1, and CMP ID NO: 23_1 showed medium tox (MT) or toxic (T) which deselects the compounds for further profiling.

### Example 5: Testing In Vitro Efficacy of Full Length Antisense Oligonucleotides and Metabolites Thereof, Targeting RTEL1 mRNA in Human MDA-MB-231 Cell Line at Different Concentrations for a Concentration Response Curve

It is known that antisense oligonucleotides are metabolized in the cell. In this experiment, full-length oligonucleotides and their metabolites from the 3′ end (i.e. n−1, n−2, n−3, n−4, n−5, and n−6) are tested for their in vitro potency of reducing the RTEL1 mRNA target.

Human MDA-MB-231 cell line was purchased from ATCC and maintained as recommended by the supplier in a humidified incubator at 37° C. with 5% CO₂. For assays, 3500 cells/well of were seeded in a 96 multi well plate in culture media. Cells were incubated for 24 hours before addition of oligonucleotides dissolved in PBS. Highest screening concentration of oligonucleotides: 50 μM and subsequent dilutions in 8 steps. 3 days after addition of oligonucleotides, the cells were harvested. RNA was extracted using the PureLink™ Pro 96 RNA Purification kit (Thermo Fisher Scientific) according to the manufacturer's instructions and eluted in 50 μl water. The RNA was subsequently diluted 10 times with DNase/RNase free Water (Gibco) and heated to 90° C. for one minute.

For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: RTEL1_Hs01548056_m1 [FAM-MGB] and endogenous control GUSB_Hs99999908_m1 [VIC-MGB]. All primer sets were purchased from Thermo Fisher Scientific. The relative RTEL1 mRNA level is shown as percent of control (PBS treated samples) and IC₅₀ determined using GraphPad Prism7.04.

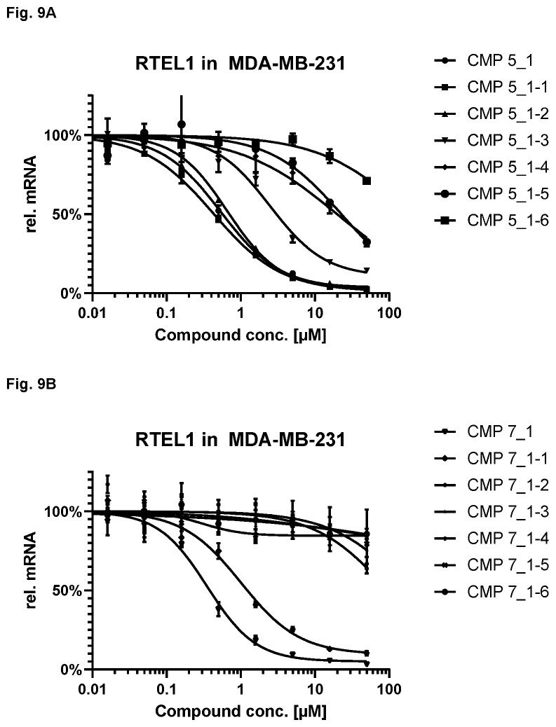

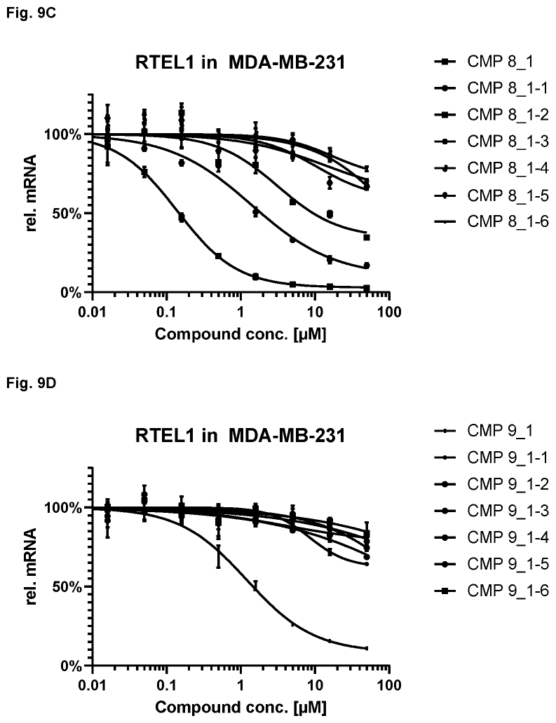

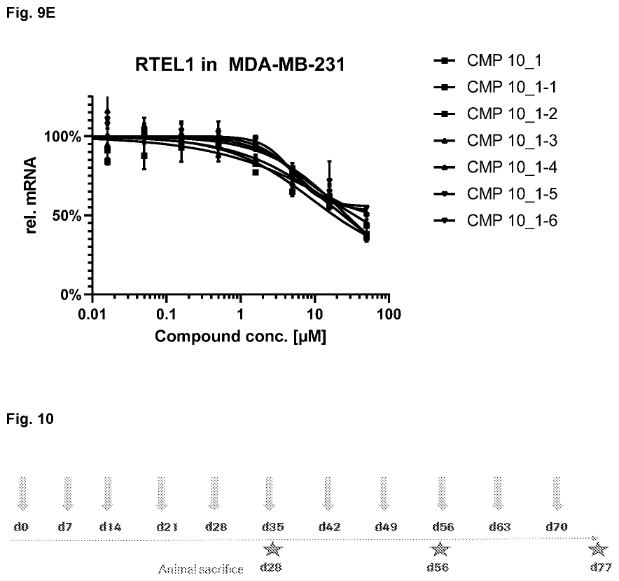

The results are shown in FIG. 9 and in Table 13

TABLE 13
In vitro potency of full-length
oligonucleotides and their metabolites

| | | | | RTEL1 |
|---|---|---|---|---|
| | | | | mRNA |
| | | | | level |
| | | | IC₅₀ | (%) at |
| | | | in | 50 μM |
| | | | MDA- | in |
| | SEQ | | MB- | MDA- |
| CMP- | ID | | 231 | MB- |
| ID | NO | Compound | [μM] | 231 |
| 5_1 | 5 | TTacatactctggtCAAA | 0.6 | 2 |
| 5_1-1 | 40 | TTacatactctggtCAA | 0.4 | 3 |
| 5_1-2 | 41 | TTacatactctggtCA | 0.7 | 3 |
| 5_1-3 | 42 | TTacatactctggtC | 2.5 | 13 |
| 5_1-4 | 43 | TTacatactctggt | — | 33 |
| 5_1-5 | 44 | TTacatactctgg | — | 33 |
| 5 1-6 | 45 | TTacatactctg | — | 72 |
| 7_1 | 21 | GAattttacatactctGGT | 0.4 | 3 |
| 7_1-1 | 46 | GAattttacatactctGG | 1.0 | 11 |
| 7_1-2 | 47 | GAattttacatactcTG | — | 62 |
| 7_1-3 | 48 | GAattttacatactcT | — | 72 |
| 7_1-4 | 49 | GAattttacatactc | — | 72 |
| 7_1-5 | 50 | GAattttacatact | — | 79 |
| 7_1-6 | 51 | GAattttacatac | — | 84 |
| 8_1 | 22 | GAAttttacatactctgGTC | 0.1 | 3 |
| 8_1-1 | 52 | GAAttttacatactctgGT | 1.4 | 16 |
| 8_1-2 | 53 | GAAttttacatactctgG | 3.1 | 34 |
| 8_1-3 | 54 | GAAttttacatactctg | — | 68 |
| 8_1-4 | 55 | GAAttttacatactct | — | 67 |
| 8_1-5 | 56 | GAAttttacatactc | — | 68 |
| 8_1-6 | 57 | GAAttttacatact | | 77 |
| 9_1 | 23 | CAAaaaacagtaggTCC | 1.2 | 11 |
| 9_1-1 | 58 | CAAaaaacagtaggTC | — | 64 |
| 9_1-2 | 59 | CAAaaaacagtaggT | — | 73 |
| 9_1-3 | 60 | CAAaaaacagtagg | — | 68 |
| 9_1-4 | 61 | CAAaaaacagtag | — | 78 |
| 9_1-5 | 62 | CAAaaaacagta | — | 83 |
| 9_1-6 | 63 | CAAaaaacagt | — | 79 |
| 10_1 | 24 | Gagggaggtggag^{m}cgTT | — | 46 |
| 10_1-1 | 64 | Gagggaggtggag^{m}cgT | — | 38 |
| 10_1-2 | 65 | Gagggaggtggag^{m}cg | — | 39 |
| 10_1-3 | 66 | Gagggaggtggag^{m}c | — | 34 |
| 10_1-4 | 67 | Gagggaggtggag | — | 47 |
| 10_1-5 | 68 | Gagggaggtgga | — | 51 |
| 10_1-6 | 69 | Gagggaggtgg | — | 54 |

As can be derived from Table 13 and FIG. 9a, the full length oligonucleotide CMP 5_1 has an IC₅₀ of 0.6 μM. Advantageously, the first and second metabolite (CMP 5_1-1 and CMP 5_1-2) retains similar target knock down activity with an IC₅₀ of 0.4 μM and 0.7 μM respectively before further metabolites (n−3 to n−6) show reduction in maximum efficacy and potency. For all other compounds, already the n−1 and n−2 metabolites show significant loss of potency and/or maximum efficacy as compared to their respective full length sequence compound.

### Example 6: In Viva Study of CMP 5_1-GaINAc Antisense Oligonucleotide

### Study Schedule

The study is run in HBV-infected PXB-Mice®, a chimeric mouse model with stable human hepatocyte engraftment (PhoenixBio) for a total of 77 days with 3 groups (vehicle, CMPD3_1_GaINAc, CMPD5_1_GaINAc) consisting of 20 animals each. Animals are dosed weekly using 10 mg/kg of compound or equivalent volume of vehicle. 5 animals of each group were sacrificed at day 28 and day 56, respectively. The remaining animals at day 77 after which the bioanalysis was performed on the livers of the animals. The study protocol is shown in FIG. 10.

### Methods and Materials

GaINAc conjugates for CMP 3_1 and CMP 5_1 were generated by methods known in the art (see e.g. Javanbakh et al. Liver-Targeted Anti-HBV Single-Stranded Oligonucleotides with Locked Nucleic Acid Potently Reduce HBV Gene Expression In Vivo. Mol Ther Nucleic Acids. 2018 Jun. 1; 11:441-454. doi: 10.1016/j.omtn.2018.02.005. Epub 2018 Feb. 23. PMID: 29858079; PMCID: PMC5992345). The produced conjugates are shown in FIG. 1 and FIG. 4, respectively.

### Animals and Handling

HBV genotype C-infected human liver-chimeric uPA/SCID mice (PXB mice; donor hepatocytes BD195 (Corning Incorporated)) were purchased from PhoenixBio Co, Ltd. Animal handling was done on site by PhoenixBio Co, Ltd. in Higashi-Hiroshima, Japan. Male mice aged at least 20 weeks at dosing start (day 0) with a weight of 18 g or more as determined on the day prior to dosing start (day −1) were selected for the study. Mice had been infected with HBV genotype C (Code No.: PBB004, Lot: 180118, PhoenixBio Co., Ltd.) at least 6 weeks prior to dosing start and showed serum HBV-DNA levels of >1×10⁶ copies/ml as determined by qPCR on day −7. Group randomization at study start was determined on day −1 based on the arithmetic mean values for body weight and geometric mean values for blood h-Alb concentration and serum HBV-DNA concentration.

All doses of 10 mg/kg were calculated based on the individual body weights of the mice which are taken prior to the administration on the days of dosing. All subject mice received an injection of the dose formulation into cervical subcutaneous tissue on the upper back on days 0, 7, 14, 28, 35, 42, 49, 56, 63, and 70 using disposable 1.0 mL syringes with permanently attached needles (Terumo Corporation).

On the respective sacrifice days, target animals were anesthetized with isoflurane anesthesia and sacrificed by cardiac puncture and exsanguination. Livers were extracted and the left lateral lobe divided into pieces of approximately 100 mg. Exact weight was recorded and liver samples were snap frozen in liquid nitrogen and stored at −80° C. until processed further.

Animals with signs of morbidity or weight loss of >20% of the initial body weight were sacrificed as required. All prematurely terminated/found dead animals as well as mice with thymoma/lymphoma on sacrifice were excluded from analysis.

Quantification of RTEL1 mRNA Expression

Liver tissue samples were kept frozen until lysed in MagNA Pure LC RNA Isolation Tissue Lysis Buffer (Product No. 03604721001, Roche) and RNA extraction continued using the MagNA Pure 96 Cellular RNA Large Volume Kit (Product No. 05467535001, Roche) on a MagNA Pure 96 Instrument (Roche) according to the user's manual and RNA concentration adjusted with water.

For gene expressions analysis, One Step RT-qPCR was performed using qScript™ XLT One-Step RT-qPCR ToughMix®, Low ROX™ (Quantabio). The following TaqMan primer assays were used for qPCR: Hs01548056_m1; Hs00249680_m1; Hs00249668_m1 and reference genes GAPDH_Hs99999905_m1; PGK1_Hs99999906_m1 and GUSB_Hs99999908_m1. All primer sets were purchased from Thermo Fisher Scientific. The relative mRNA expression levels are shown as percent relative to saline treated control group.

The results are shown in FIG. 11. The analysis shows approximately 80% knockdown efficacy with CMP3_1_GaINAc at weekly dosing of 10 mg/kg and ca. 95% knockdown efficacy with CMP5_1_GaINAc at weekly dosing of 10 mg/kg.

### Example 7: In Vitro Study in Primary Human Hepatocytes

### Methods and Materials

### Cell Culture

Primary human hepatocytes (PHH) isolated by the collagenase perfusion method from chimeric urokinase-type plasminogen activator/severe combined immunodeficiency (uPA/SCID) mice with humanized livers were obtained from PhoenixBio (Hiroshima, Japan). PHH were plated on type I collagen-coated 96-well plates at a concentration of 7×10⁴ cells per well in modified hepatocyte clonal growth medium (dHCGM). dHCGM is a DMEM medium containing 100 U/ml Penicillin, 100 μg/ml Streptomycin, 20 mM Hepes, 44 mM NaHCO₃, 15 μg/ml L-proline, 0.25 μg/ml Insulin, 50 nM Dexamethasone, 5 ng/ml EGF, 0.1 mM Asc-2P, 2% DMSO and 10% FBS (Ishida et al., 2015). Cells were cultured at 37° C., in a humidified atmosphere with 5% CO₂. Culture medium was replaced every 2 days until harvest, except over the weekend.

HBV genotype C at multiplicity of infection (MOI) of 40 was then added to the cells in 96-well plates with a final concentration of 4% PEG 8000 (Sigma-Aldrich). Infected cells were incubated for 20 h at 37° C., and then washed two times with PBS to remove the HBV inoculum and refilled with complete media. At day 4 post-infection, cells were treated with different concentrations of LNA. Medium was changed with new LNA at day 6 and day 8 post-infection. Phosphate-buffered saline (PBS) was used as a “no drug” control (NDC). Supernatants and cells were harvested and used for HBV marker and target knockdown analysis on day 19 post-infection.

Cytotoxicity of the LNAs was assessed using Cell Counting Kit 8 (Sigma-Aldrich) according to the manufacturer's protocol and expressed as % cytotoxicity vs. NDC.

### RNA Extraction and Real-Time Quantitative PCR

Intracellular mRNA was extracted from cells using a MagNA Pure 96 robot and the MagNA Pure 96 Cellular RNA Large Volume Kit (Roche) according to the manufacturer's protocol. Quantification of relative total HBV, RTEL1, pgRNA, and β-Glucuronidase (GusB) endogenous control mRNA was performed in technical duplicates using a QuantStudio 12K Flex (Life Technologies) using the TaqMan RNA-to-Ct 1-Step Kit (Applied Biosystems, #4392938). The mRNA expression was analyzed using the comparative cycle threshold 2-ΔΔCt method normalized to the reference gene GusB and to no drug control. TaqMan primers and assay IDs were as follows: RTEL1, Hs02568623_s1; Total HBV RNA Pa03453406_s1; HBV pgRNA AILJKX5. As reference gene GusB, Hs00939627_m1 was used. All primer & probe sets were obtained from ThermoFisher.

The results for 25 μM of antisense oligonucleotide are shown in Table 14 which, inter alia, includes information on cytotoxicity of the cell and RTEL1 mRNA knock down. CMP10_1 shows a high cellular cytotoxicity and poor efficacy in targeting RTEL1 mRNA. In addition, CMP9_1 shows the second poorest knock down of RTEL1 mRNA with 57% remaining RTEL1 mRNA.

TABLE 14
In vitro study in primary human hepatocytes (25 μM of antisense
oligonucleotide)

| | | % RTEL1 | % | % total |
|---|---|---|---|---|
| LNA | % | mRNA vs. | pgRNA | HBV-RNA |
| @25 μM | cytotoxicity | NDC | vs. NDC | vs. NDC |
| 5_1 | 4 | 17 | 70 | 72 |
| 5_1_GalNAc | 5 | 15 | 57 | 46 |
| 7_1 | 1 | 24 | 83 | 76 |
| 8_1 | 3 | 13 | 74 | 81 |
| 9_1 | 2 | 57 | 103 | 94 |
| 10_1 | 30 | 101 | 61 | 53 |

The results for 5 μM of antisense oligonucleotide are shown in Table 15. CMP10_1 shows poor efficacy in targeting RTEL1 mRNA. In addition, CMP9_1 again shows the second poorest knock down of RTEL1 mRNA at 79% remaining RTEL1 mRNA.

TABLE 15
In vitro study in primary human hepatocytes (5 μM of antisense
oligonucleotide)

| | | | % | |
|---|---|---|---|---|
| | | % RTEL1 | pgRNA | % total |
| | % | mRNA vs. | vs. | HBV-RNA |
| LNA@5 μM | cytotoxicity | NDC | NDC | vs. NDC |
| 5_1 | 4 | 47 | 77 | 80 |
| 5_1_GalNAc | 2 | 29 | 62 | 72 |
| 7_1 | 0 | 40 | 84 | 92 |
| 8_1 | 1 | 28 | 78 | 101 |
| 9_1 | −1 | 79 | 146 | 130 |
| 10_1 | −4 | 106 | 175 | 116 |

### Example 8: In Vivo Proof of Concept for CM P5_11-GaINAc

### Study Schedule

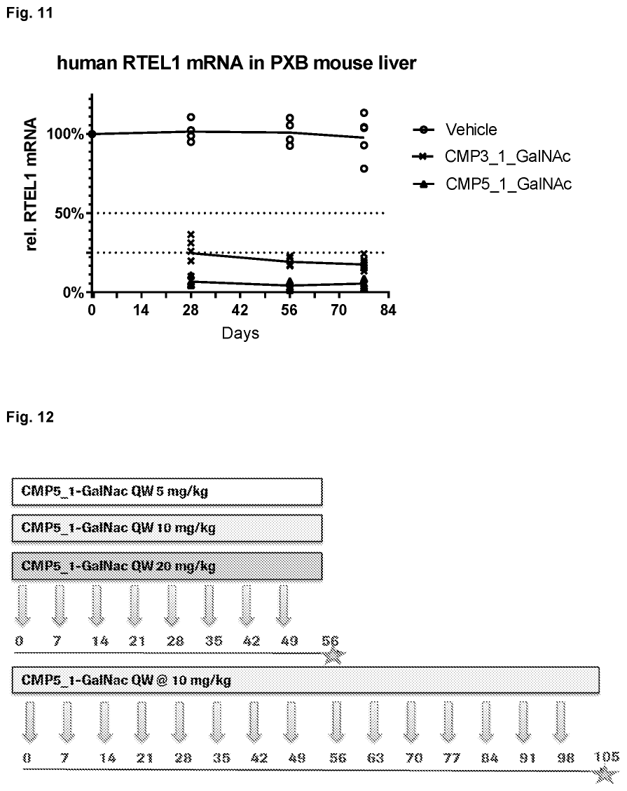

The study is run in HBV-infected PXB-Mice®, a chimeric mouse model with stable human hepatocyte engraftment (PhoenixBio) with sacrifices at day 56 and day 105. CMP5_1-GaINac is evaluated after 8 weekly dosings of 5, 10, and 20 mg/kg (day 56, see FIG. 12). Additionally, CMP5_1-GaINac is also evaluated after 15 weekly dosings of 10 mg/kg (day 105). For each group, there was a separate vehicle control arm receiving the same volume of vehicle as the treated animals. All groups had a size of 6 mice at study start. The bioanalysis was performed on the livers of the animals.

### Methods and Materials

### Animals and Handling

HBV genotype C-infected human liver-chimeric uPA/SCID mice (PXB mice; donor hepatocytes BD195 (Corning Incorporated)) were purchased from PhoenixBio Co, Ltd. Animal handling was done by LSIM Safety Institute Corporation in Uto, Japan. Male mice aged at least 25 weeks at dosing start (day 0) with a weight of 18 g or more as determined on the day prior to dosing start (day −1) were selected for the study. Mice had been infected with HBV genotype C (Code No.: PBB004, Lot: 180118, PhoenixBio Co., Ltd.) at least 6 weeks prior to dosing start and showed serum HBV-DNA levels of >1×10⁶ copies/ml as determined by qPCR on day −7. Stratified randomization was performed at study start on day −1 based on body weight and blood h-Alb concentration as well as serum HBV-DNA concentration.

All doses were calculated based on the individual body weights of the mice which are taken prior to the administration on the days of dosing. All subject mice received an injection of the dose formulation or matched vehicle into cervical subcutaneous tissue on the upper back. Animal sacrificed on day 56 were injected on day 0, 7, 14, 21, 28, 35, 42, and 49 (see FIG. 12). The dose of 20 mg/kg/week was given in two consecutive injections of 10 mg/kg to avoid overloading the ASGPR in the chimeric livers. Thus, these mice were dosed on days 0, 1, 7, 8, 14, 15, 21, 22, 28, 29, 35, 36, 42, 43, 49, and 50. Animals sacrificed on day 105 had administration of 10 mg/kg on day 0, 7, 14, 21, 28, 35, 42, 49, 56, 63, 70, 77, 84, 91, and 98 (see FIG. 12) using disposable 1.0 mL syringes with permanently attached needles (Terumo Corporation).

On the respective sacrifice days, target animals were anesthetized with isoflurane anesthesia and sacrificed by cardiac puncture and exsanguination. Livers were extracted and the left lateral lobe divided into pieces of approximately 100 mg. Exact weight was recorded and liver samples were snap frozen in liquid nitrogen and stored at −80° C. until processed further.

Animals with signs of morbidity or weight loss of >20% of the initial body weight were sacrificed as required. All prematurely terminated/found dead animals as well as mice with thymoma/lymphoma on sacrifice were excluded from analysis.

cccDNA Determination by Southern Blot Analysis

Liver tissue was kept frozen until homogenization in DNA extraction buffer (50 mM Tris pH8, 5 mM EDTA, 150 mM NaCl, 1% SDS). The extract was first digested with RNase cocktail (ThermoFisher) for 30 min at ambient temperature followed by Proteinase K (ThermoFisher) for 2 h at 56° C. under light agitation. Debris was pelleted by centrifugation and DNA extracted from the supernatant by shaking three times with 1 volume UltraPure buffer-saturated phenol (ThermoFisher) followed by extraction with 1 volume of Phenol:Chloroform:Isoamyl Alcohol (25:24:1; ThermoFisher). DNA was then precipitated by adding 1 volume 100% ethanol and 60 mM (final) NaAc (SigmaAldrich) at −20° C. over night. DNA is then pelleted by centrifugation, the pellet washed with 70% ethanol and air-dried before resuspension in 10 mM Tris-HCl pH8 (ThermoFisher).

DNA content is quantified using NanoDrop (ThermoFisher) and concentration normalized to 1.5 μg/μl. 30 μg DNA are then subject to digestion with 70 U T5 Exonuclease (New England Biolabs) in 1× CutSmart buffer (New England Biolabs) for 2 h at 37° C. For each sample, equal volumes are mixed with Blue Juice loading buffer (ThermoFisher) and loaded onto a 0.95% agarose-TAE gel (SigmaAldrich). Between every (biological) group of samples, 1 lane loaded with DNA molecular weight marker VII (Roche) is added (same amount in each lane). After separation, the gel is transferred to fresh 0.2 M HCl (Acros Organics) for 10 minutes and then rinsed three times with water. Subsequently, the gel is placed in denaturing buffer (0.5 M NaOH, 1.5 M NaCl) for 30 min and rinsed once with water. Next, the gel is transferred to neutralizing buffer (0.5 M Tris pH7.5, 1.5 M NaCl) for 30 min. Finally, the gel is equilibrated with 20×SSC buffer (ThermoFisher) for 30 min. The DNA is then transferred to a Hybond-XL membrane (Cytiva) using a TurboBlotter kit (Cytiva) according to the manufacturer's instructions using 20×SSC buffer (ThermoFisher) for overnight transfer. After transfer, DNA is crosslinked to the membrane by irradiation with 1800 J UV radiation and the membrane dried.

For specific detection of HBV cccDNA, the DIG nucleic acid detection kit is used with DIG EasyHyb buffer and the DIG wash and blocking buffer set (all Roche) according to the manufacturer's instructions. The probe is generated by PCR using the DIG PCR probe kit (Roche) according to the manufacturer's instructions. A plasmid containing the HBV genotype C genome is used as template with the following primers: forward, GTTTTTCACCTCTGCCTAATCATC (SEQ ID NO: 70); reverse, GCAAAAAGTTGCATGGTGCTGGT (SEQ ID NO: 71).

Hybridization is performed with probe denatured at 100° C. for 5 min and then rapidly cooled to 4° C. at 37° C. overnight. Washes and detection are performed according to the manufacturer's instructions. Images are acquired using a FUSION FX system (Vilber) setting an appropriate exposure time to detect cccDNA in vehicle control samples. For analysis, the cccDNA band (2.1 kB size) is quantified using ImageStudio software (Licor). To account for uneven transfer, the 2.8 kB band of the DNA molecular weight marker VII is also quantified and cccDNA band intensities are normalized to the average intensity of the two bands framing the group. Individual values are then normalized to the average band intensity of the vehicle control group.

RTEL1 mRNA Analysis

Liver tissue samples were kept frozen until lysed in MagNA Pure LC RNA Isolation Tissue Lysis Buffer (Product No. 03604721001, Roche) and RNA extraction continued using the MagNA Pure 96 Cellular RNA Large Volume Kit (Product No. 05467535001, Roche) on a MagNA Pure 96 Instrument (Roche) according to the user's manual and RNA concentration adjusted with water.

For gene expressions analysis, One Step RT-qPCR was performed using qScript™ XLT One-Step RT-qPCR ToughMix®, Low ROX™ (Quantabio). The following TaqMan primer assays were used for qPCR: Hs01548056_m1; Hs00249680_m1; Hs00249668_m1 and reference genes GAPDH_Hs99999905_m1; PGK1_Hs99999906_m1 and GUSB_Hs99999908_m1. All primer sets were purchased from Thermo Fisher Scientific. The relative mRNA expression levels are shown as percent relative to saline treated control group.

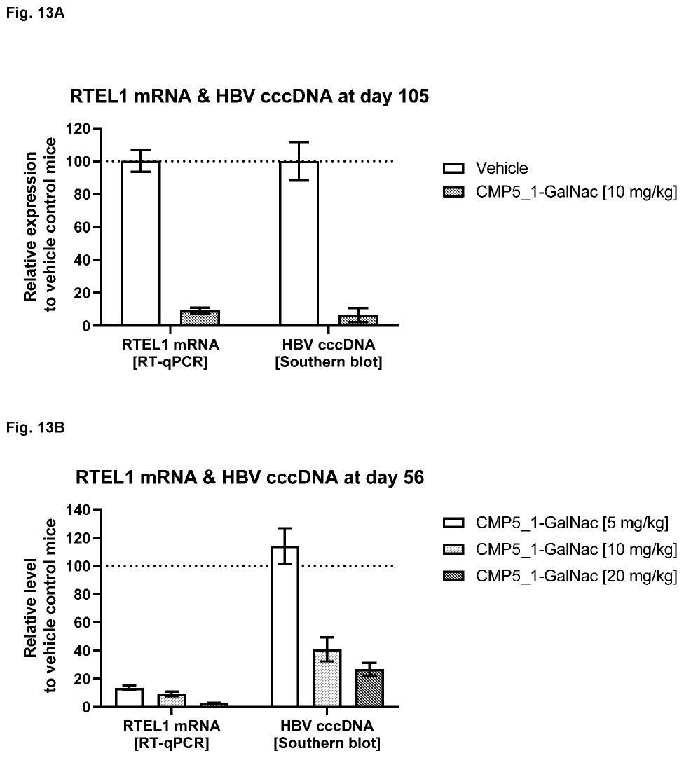

FIG. 13A shows an example of weekly dosing of CMP5_1-GaINAc for 105 days gives reduction of RTEL1 mRNA to ˜15% and cccDNA to ˜20%

FIG. 13B shows knock-down of RTEL1 mRNA of 87%, 91%, and 97% after weekly dosing of 5, 10, and 20 mg/kg CMP5_1-GaINac, respectively, at day 56. cccDNA was reduced by 59% and 73% for 10 and 20 mg/kg CMP5_1-GaINac, respectively.

Conclusions: It follows from the above Examples that CMP ID 51 displays a safe and efficacious compound for targeting the RTEL1 mRNA both in vivo and in vitro. We have here shown a favorable in vitro drug property profile of CMP ID_5_1 and we have showcased that the target of RTEL1 can be engaged very efficiently in vivo with the subsequent reduction of viral cccDNA. In addition, we have shown that by down regulating the RTEL1 mRNA a significant dose dependent effect on the viral cccDNA using hepatitis B infected humanized mouse models can be elicited. The compound 5_1 furthermore has a property of retaining activity after degradation from the 3′ end. This means that the n−1 and n−2, from the 3′ end, has a comparable in vitro potency as compared to the full length CMP ID_5_1. The n−3 degradation also still retains activity towards RTEL1 albeit with a lower in vitro potency. This profile gives the molecule a clear benefit as the metabolites of the parent molecule still elicit the wanted effect on RTEL1 mRNA.

1. An antisense oligonucleotide selected from the group of antisense oligonucleotides consisting of
(SEQ ID NO: 5)
TTacatactctggtCAAA,
(SEQ ID NO: 3)
AATTttacatactctgGT,
(SEQ ID NO: 4)
AAttttacatactctGGTC,
and
(SEQ ID NO: 6)
CTttattataactTgaAtCTC,
wherein capital letters are beta-D-oxy LNA nucleosides, lowercase letters are DNA nucleosides, all LNA C are 5-methyl cytosine, and all internucleoside linkages are phosphorothioate internucleoside linkages.

2. A conjugate comprising the antisense oligonucleotide according to claim 1, and at least one conjugate moiety covalently attached to said antisense oligonucleotide.

3. The conjugate of claim 2, wherein the at least one conjugate moiety is capable of binding to the asialoglycoprotein receptor.

4. The conjugate of claims 2 and 3, wherein the conjugate moiety is selected from one of the trivalent GaINAc moieties in FIG. 5.

5. The conjugate of claim 4, wherein the conjugate moiety is the trivalent GaINAc moiety in FIG. 5D, such as the trivalent GaINAc moiety of FIG. 5D-1 or FIG. 5D-2, or a mixture of both.

6. The conjugate of any one of claims 2 to 5, comprising a linker positioned between the antisense oligonucleotide and the conjugate moiety.

7. The conjugate of claim 6, wherein the linker comprises or consists of 2 to 5 consecutive phosphodiester linked nucleosides.

8. A conjugate selected from the group of consisting of the conjugates shown in FIG. 1, FIG. 2, FIG. 3 and FIG. 4.

9. A pharmaceutically acceptable salt of the antisense oligonucleotide of claim 1, or the conjugate of any one of claims 2 to 8.

10. A pharmaceutical composition comprising the antisense oligonucleotide of claim 1, the conjugate of any one of claims 2 to 8, or the pharmaceutically acceptable salt of claim 9, and a pharmaceutically acceptable diluent, solvent, carrier, salt and/or adjuvant.

11. An in vivo or in vitro method for modulating RTEL1 expression in a target cell which is expressing RTEL1, said method comprising administering the antisense oligonucleotide of claim 1, the conjugate of any one of claims 2 to 8, the pharmaceutically acceptable salt of claim 9, or the pharmaceutical composition of claim 10 in an effective amount to said cell.

12. A method for treating or preventing a disease comprising administering a therapeutically or prophylactically effective amount of the antisense oligonucleotide of claim 1, the conjugate of any one of claims 2 to 8, the pharmaceutically acceptable salt of claim 9, or the pharmaceutical composition of claim 10 to a subject suffering from or susceptible to the disease, wherein the disease is hepatitis B virus (HBV) infection, such as chronic HBV infection.

13. The antisense oligonucleotide of claim 1, the conjugate of any one of claims 2 to 8, the pharmaceutically acceptable salt of claim 9, or the pharmaceutical composition of claim 10 for use in medicine.

14. The antisense oligonucleotide of claim 1, the conjugate of any one of claims 2 to 8, the pharmaceutically acceptable salt of claim 9, or the pharmaceutical composition of claim 10 for use in the treatment or prevention of hepatitis B virus (HBV) infection, such as chronic HBV infection.

15. Use of the antisense oligonucleotide of claim 1, the conjugate of any one of claims 2 to 8, the pharmaceutically acceptable salt of claim 9, or the pharmaceutical composition of claim 10, for the preparation of a medicament for treatment or prevention of a hepatitis B virus (HBV) infection, such as chronic HBV infection.


SEQUENCE LISTING (71 sequences)

SEQ ID NO: 1 - DNA, 38444 bp, Homo sapiens
  source 1..38444: mol_type = genomic DNA
agtcagccctgctgccagccagtgccgggtgctggggactcagggaggcccgccgggaccactgcgggacagtgagccgagcagaagctggaacgcaggagaggaaggagagggggcggtcagggctctcaggagccgggtcctgggcaaggcgcagccgttttcaaattttcaggaaagcggtcggctcacactcgagcagtaaaaagatgcctctggggaggaggcccgtgcagctctccgggcaatggtggtggctcggcctagagaggcggtagtggaacgcagaccctggtgggggaatgacatcaagggaggagacgggcgggaccccagatttctgcctgtgggcgatggaagtgaggttcactggccagcggagccggacacagaacgcgcaaaacgccgtgtaggcctggaggagccgaagagcaggcggaccccctccgcgggggaacagtttccgccgggagcacaaagcaacggaccggaagtggggggcggaagtgcagtgggctcagcgccgactgcgcgcctctgcccgcgaaaactctgagctggctgacagctggggacgggtggcggccctcgactggagtcggttgagttcctgagggaccccggttctggaaggttcgccgcggagacaagtgagcagtgagtcgcagtgaccctacaagtggttcttttacccgagcggctcgtaggcgcgttgcggtttttcgaaactacagctcccggcaggccccaagccgccctcggggccgcgggtcggcggattggccgcgctgcattttgggacctgtagtttcctgcgctcgtggcgctggcgccgcggcgttggctgagcccttgaccggggctggagggaagggccgacattcagtgtgtccgcgtctgttctgttagtcccagttcccgggcgggattgaggcttagagaagttgagtgatttgctgagggctgcacgggttggcatcccggcatgctctttcgctactttggctgcatctggttgcccacccgggcggatggggaatggactccagccagccaggagggcagagggctggagaggcagggccggaggttcagaccctccgctctgacgttgcgcctggtgaggccgggaggggtgccgcttgcctcttcagccctcacgctcttgtggaagtcgcggaattactgcaggcggaacttgcagcactgtgggcgtcttttccagagaaggacggagttgtggggcgggaggataaggcaaggcccagccacttcgcatcttcgccccgccagctcctcgagatgggatataccagggttgctctccaaccctctccgcaggagggactgatggaaacgcctgggaaagtagcccggtacccacaaaggctgtctacaaacagagtcttactgtctttcccaggtctgtgccatagggattctcgaagagaacagcgttgtgtcccagtgcacatgctcgcatcgcttaccaggagtgcccgagaccctaagatgttcggagtggttttttcgcacagacccgaatagcctgcccctcagccacgctctgtgcccttctgagaacaggctgatatgcccaagatagtcctgaatggtgtgaccgtagacttccctttccagccctacaaatgccaacaggagtacatgaccaaggtcctggaatgtctgcagcaggtagagcacaggccccgaggaaaggactgcgggtgggtggagcttcagccaggacggggtgtgcttccctctcccggcccattccagccaggcccctccgggccagaggcagcgtctgtcataaaaagggctggtgttccaggtggggtcagagagaggattgacaagtaaaaacgatcgtcctttgaagggggccggcccctccacacctgtgggtatttctcatcaggcgggacgagagactgagaaaatgaataagacacagagacaaagtatagagagaaaagtgggcccaggggaccggcgctcagcatacagaggacctgcaccggcaccagtctctgagtttcctcagtattcattaattactattttcactatctcagcaagaggaatgcggcaggacagcaaggtgatagtggggagaaggtcagcaagaaaacgtgagcaaaggaatctgggtcacaaataagttcaagggaaggtactatgcctggatgtgcacgtaggctagttttatgcttttctccacccaaacatctcggtggagtaaagagtaacagagcagcattgctgccaatatgtctcgcctcctgccacagggcggcttttctcctatctcagaattgaacaaatgtacaatcgggttttataccgaaacattcagttcccaggggcaggcaggagacagtggccttcctctatctcgactgcaagaggctttcctcttttactaatcctcagcacagacccttcacgggtgttgggctgggggactgtcaggtctttcccatcccacgaggccatatttcagactatcacatggagagaaaccttgggcaatacccggctttccagggcagaggtccctgcggctttccgcagtgcatcgtgcccctggtttatcgagactggagaatggcgatgacttttaccaagcatactgcctgtaaacatattgttaacaaggcacgttctgcacagctctagatcccttaaaccttgattccatacaacacatgtttctgtgagctcaaggctggggcaaagttacagattaacagcatcttagggcaaagcaattgttcagggtacaggtcaaaatggagtgtgttatgtcttccctttctacatagacacagtaacagtctgatctctcttttccctacagtccttgagggtgacagacttaggagtgccttgggggcctctctgaggagcagctgatattcacgggtcaggaggaagcatttccattagaggggcagccggtggccagcctcacttggaaggtctttgaacctcgggggtgcagggaggtggcagtggtgcaggttgccttctcctgggttccttgaggtgccctcttgtacccggctcacacccttcccctccccgagtttcctgctcaggttcccgtctgagagcttgtatgtaggacgtcagataggacagcataaatgtttggatccagaaacgcagaacagtttcctattttgagacttgacacctaattagtcatcttactatttaagctgaaaaatagtgtcgtgttttgggtaacgttctgcaaatcgtttgctaatggcggctgagttgcttcacgccctttagggcaagagtgggacttgcctgtggacttctccgcggtcccacagggctctcgccacctggcagtggcctctgcatctgcaaagagctgcccgctggctgccgaagcttgtctcagggcagcttgtgtggcctcgcctcttcctggcttccccgtaacccttgctccgaactccgttcagaaggtgaatggcatcctggagagccctacgggtacagggaagacgctgtgcctgctgtgcaccacgctggcctggcgagaacacctccgagacggcatctctgcccgcaagattgccgagagggcgcaaggagagcttttcccggatcgggccttgtcatcctggggcaacgctgctgctgctgctggagaccccataggtgaccctagttcccaggcctctcctggcctcctgtggggatggttggcaagggatggcgctgagggtggggtgggcccatggggactcctgccgtctctcaagcagaactcaaggagaattttttagctgctgtataatttctcgccatcgtgggtgtaaacctagggttgggcttttttgctgaattagggcacggcagatgcccacttcacccatttttgataaaccagtatctggggtgtcagattcttggctgtctgcagggccgagttagccgaatgccacctgcctttgatacgtgagaacgttgtctgagaaccgtgacttctgtgcttgcttgtgtctggtcagcttgctacacggacatcccaaagattatttacgcctccaggacccactcgcaactcacacaggtcatcaacgagcttcggaacacctcctaccggtgggtcagacgagtttacacctgtctcggggtcctcaagagaaccagcttggcatggtgctgagtccacagccccatgctgtgctgtggtggagggtggtggtctttctagacgctcccccgaagtgtgcagagcgctggtgcccaggggtggggtgcggcctgggctgcctccaatgcccattacttgtgaggaagcagctttgcatctgtgtgctgaccttgggcgggcgtcctgagctcctcgcaggtgctgttgtagcagctgtgcagtaggtcagggctggcccccagtgcagctttgcacatgaagtaggaggaggccctgctgcttgtcagagcccagcagagtcttggtgttctgtcgggttcctgtggccggaccagtggcagggtgctgtggaagctgtcgaatctcctccctctgtccagtacccccgctcgtcttctagctccctcctacgcccgggccacgtttcagttatgctcacttcctctgaccgccgaggctcctgcgtgtctccatacagctcacgctgcagggccacgctgtgggtgttggagacagctcctcctcgacccacggtgctctctcccaccaggcctaaggtgtgtgtgctgggctcccgggagcagctgtgcatccatcctgaggtgaagaaacaagagagtaaccatctacaggtaggctcctgggctcccgctccggctcagtgtccgacaggcgagtgctgctgggtgtccagagccccaggctgcgctcccgctgggctagggtttgaagttcactgggggactgcaggggaggacctggtgggggtggggactggcttcggtcctttcttggccgtgcttcagctgcgcactctgcccttcctcccacagatccacttgtgccgtaagaaggtggcaagtcgctcctgtcatttctacaacaacgtagaaggtacaagcagctgggtgggaccagggtcgggttggagtgtgtgcagcctctcagggtggagctcagtggtgtcacagcctggttgtgcttgcccggtggggcggccagtgcggccatgtacctgggccctgtcttctgactcggggccacccatgttagacttctgtgtggaagagctcacacagtggtctgagacagccagccggcaagactgcctctggctggtgcctggggccttggattttgggaaggctccctccatttcctgatgagagggtctccctgcacctaacctgctggtgcaaacagtaggggttttgctgaacaccggctttctcttcggggactttgttgcttgcccagcagcaggtgctccagtgaccggccctcataccatcttgggagggtgtcctggaagccgtgtctggcctcccgcgaccctgccccgtgtgtctttttcctgtgctgaccttgctgcggaaaattatggccctgagtgtgactccaggctgagtcctgtgggtccaacacgggatgccttggggcctcttctggagacgggatgtgagtgacaggagccggccggggcagcttgccctgtgactgcacgtggccacagcctgtgagggccgggggtgcttctccacccacgtggctgcccctcgggtatgtcaagggcttctggggctcatcacggggtcctagagacagtggcagggtgcacccccgttggctgcccttacagtttctgtgacctgagggtggcatctgtgcagtcggcgcggtctgtgcttctgtgggatcagggttccctctgtttcctgcctcagttggggctcaagcctcaggtgaggtggccccggagcactcagaaggcatcggcggtcctgtgggctgctttctgcactcacgtttgctgagtgctcagtgtgccaggactgaggaccctgaagctgctcttgtatttagggcggcgctcccctggcagagactgagccaggtggtcccgcatgacccactaccaggcgtttctgggccctggcccttggagggacagggtgggcggaacatgggcctgcagggaggctcccgcttactggaggcatgtgctgtgttgctggagacatcctctgtgttgcttcttgttcgctgtggtttttggtctggtggcaccaaggaccctcagtcatcttgatgtgtggttgtccaggcctttttgttggtcctaagaaggggctctgcctttgtgcccccaggttccctgacaggagctgccggctcgtcccggtgatgcctgcaggacgtgactctgggacggggggttgggcagatgtgctgatggaaattctcaagcaggcgtcatttccgaggtcctcacctggatttccaggacaggagtgcctgctgggtgtccccagtcccatgcagcgggggtccttgggatagcatggaacgctgagcatgggcctggccggccgtggtcctggacaagggcagtgccccggtggctgctgggcctgggacctggtggggacgctgggcctggtacctggtggggatgctgggcctgggacctggtggggaggcctctgactgcctcctggtgctgcttccgtctgtgttaggcctctgggtattggggcccccatctgtctcctcctccaggcctgtggactcagaccaggaagacacaggccagcccctgcctgtcccccttggcttgggctctcactgcccgacctggcgggaggttgcctagccgtgaaccttcgcaccctgtctgccaccggacaggctgtgagggggtgtctgcagcacctgcaccggcctgagcatcttcagagtgggctgcagctcctggaggggtctgagaggaagggaggcaggtattttgggcgaatgaggagacagctggagagctggcacccttcctggcctgcgtcctgtgaggactctggttggggacagcaagcttggggtcagcctggggcagagcctctgggacggccccgcccctcgtgccccttcccctcgcagctcctgtcctcgccccgccctcagctctccgccaggcaaggtttggcaagtgccgctgtgcggcagtgcctgctgattggctggtctgttgctatggtgctgcccaggggtgtgcttttcctcccctgccttccctgctatccctgggagtatctggggttgggtcatcgctggtgtgtgtgagtgtgtgtgtgtgtgtatgtgcacgtgtgcatatgtgtgcgcttctggcctctgcagctgagtcctggccctcggggggcctggcacctcctggggacaggcacaaagcagccatgatggagtcgggagctgggggaggccccattgccccacgtggctgccctgtgactctggggtgcttgttagaagaggtatctggttctgtctgtgtttaagcaactccctaaggaattcttgtggttccagtttggggggcctgtactgtagaggcaagggaggggcaggacatcccccagactctgacttctgaagccttttctgcccggggcctctccgccagtacaggcagtgtcctttgccagggctgccatgctgcagaggggagtgggccactgtttagcccaggaaaacctggctctcccttagctggaagttctgggcctgttgtggttggcagggaagctgagtgacggtgctaatcacaggggcacctgcaggggtttgtgggagatgcctctgtgggttggggcgataggctgaggggctgttcttccctgccctgaggagggctgagtgtagccgccactcctgtcctgtcttgggctgtctcggagaggatgcgtagaaccctcgggatcctgctggcctccgtctggtccaccctgaacctcaggccttctgggggcagaggaggattccctcaggatcactcgggtgggggcctctcttgggcacctgagaccctcagtgggtgctttgtggcgcgttcacggttggtgggggacgcccagccctgcccgccgtgtaggagccgttctgtcctgggcatccccctgtggtctgggacttagtggaccctgagggtgtgtgtttacccctgcctcacacctgcagaaaaaagcctggagcaggagctggccagccccatcctggacattgaggacttggtcaagagcggaagcaagcacaggtgagacccctcagtgaggccacgaccactgtccttccatggcccagctctcctgtgacctgtggaggcccggatatatttcttcacttttctttgttcctttttaaattatgaaactaaccaccattcagtacgaaaaagtttaagcagctctgaggaagatagagtaaaaaattgtctccctcttccctggccctcagccatccccggtggccaccgtggagtgtggacggagccctgcaggcctgtgtctgtgcggaagcacgcgcagttttgtctgcacagactgtcctgcagttggctgttttcactcagcgttgtgggtatagcttcccatgctggtgctggcagctcggccttgttcttttgaggacagcagatgtctcctatgtctacctcttacagcttcagagattcaagttataataaagctcttcttatattgagggggaaacctccctcccccttttttttgaaacagggtctcgctctgctacccaggctgcagtgcagtgtcacagtcttggctcactgcagcctcagcctcccaggctcaagcgattttcccacctcagcctcccaagtagccgggactgcaggcacacaccaccatgcctggttaatttttgtattttttgtacagacagggtctcactctgttgctcaggccagtctcctgagctcgagagttccacctgccttggcctcccaaagtgctgggattacaggcgtgagaccccatgcctggccagctctttttttttttttttttttttttgagacggagtctcgctctgtcgcccaggctggagtgcagtggtgcgatctcggctcactgcaagctccgcctcccgagttcacgccattctcctgcctcagcctcccgagtagctgggactacaggtgcccgccaccacgtctggctaattttctgtatttttagtagagacggggtttcaccgtgttagccaggatggtctcgatcttctgaccttgtgatccgcccacctcggcctcccaaagtgctgggattacaggagtgagccaccgcgcccggcccagctctgctttttcttagtggttctgcgttgtgtttgtttctatccaggaatagggttggttttacttttccatcgagtttttaaagagacgacgatttacatggtcggaaactcacgaggactccccatcccttggtcggaaactcacatggactccccatcccttggtcagaaactcacgtggactcccatccatcccaggcagcagcttcccacctgggccctacgtgcaggatgagggctccttccgggtcagaagacatggcggcctcggggcaccgtcccctgcatggggtgctcacaggatcttctcctctctccttcccagggtgtgcccttactacctgtcccggaacctgaagcagcaagccgacatcatattcatgccgtacaattacttgttggatgccaaggtgggggctcagtcctgtagctgacgactcctgatgtccaggggtgtccctgggcttgggaacagctgtccgagcctttgctgcttcagggccttagatcagcaggcctgggtgggaggactcacctctgtcactgggcaggggctcaacctggccagacacacttgtgagcagccccaggccacaggtcagttttctgagcagtctgggagcgggcaggctggtgggagtgaggagagacctccaggctgtggtccataggccagtgcccgctcttgatcctgacagctcaggttctctccttcacgtcaggccatgggaggcaccgagaacacaggaagcccactgactcccctcttcccagcgcgtgcccggccccacactcactccccctcccagcatgtgcccggcttcacactcactcccctcttcccagtgcatgcccggccccacactcactccccccacagcatgtgcccggcctgacactcactcccctcctcccagtgtgtgcccagccccactcccttccgccccgtgtgcccagccccacgctcactccccccgccagcatgtgcccggccccacactcaactcccctcctcccagtgtgtgcccggccctgctgccctcctccccatgtgccctgcttttgtgccccacactttttacttagtgcaggtgggatcacacgccacgggtcaatggtttgtgtgttcacgtgacgatggcgtggtgacgtttccagatcccgtcgttggttcgctcattctcggggtgtatatttattgagagctcatcatgctgggtgctattccaggcatagcaagactggcttcactcacatggagctttgattctagtggtggggacaggtggacagcaaaagagtaagcacgtgagctgatgatactgaagggaaatagagcagagggaggaggcggagaccgagccaagcgggcccaagtgcgatgtcggcgggaggtggggaatgctggtgggtctgaggggagcctcagcaggtgcagcagagcaagggaagaggtgagtgggggcggctggggggccgactcctgggaagctgtagcagaaccccacagagagctggtgaggtttgccgtggttgtgggtgactcggtgctttgagccctggctgcccctgggaaccatctggagagcttctaacccaaccaggcccctccctgggacagttatatcacagctggtaagccgagtctaacactttcacggaaacgcagaagatctaaaacagcaagatgaccgtgaagaagaacagagctggaggactcacctcgctggtttcaagactcctctaaagctgcaggagtggaggtggagatggcccagctcaggcacaggcctgcaggccatggagaaggcagcaagctcaagctgacccacacgcatgtggtcattgttttttttttcagttggaatctcactctgtcacccaggttggagtgcagtggcaccatctcggctcactgcagcccccgcccctaggttctagcgattctcccacatcagcctcccgagtagctgggattacaggcgtgcgccaccatgcctggcccttggtgattgttttttgacaaacatgccaatttaattgagagaggaaatgaaggttgatttctggttttctgaaaaaatggtgctaagaacagctggatatctgttcggaaaacagtgaatcttaactcttgttttaccctgtataaacctaaatgtaaaagctaaactaaaagttatagaaaggaacatgggggaggtctttgcaactttggggtaggcagagatttcttagtatggatacacaaggcactagccatgaagaaaaacattaaaatttagacttcaccaaaatttaaagcttcaactctgtggaagagttgagaaaatgaaaaagcagttaaagaaagggagaaaatacttctttcaaaggacttaaaaaattttttcagccctcctctgatttgaaaggacctttgaccagagtatgtaaaattctcccataactaagcaaacaacccacttaaccactgggaagggatctggacagacgtttcaccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcaccaaggtggatggaatgaccagttgagcacatggaaagtcgcccagcatctccagtcataggagaaggcagattaaagccacggggagccgacactgtggtcccactggcatggctgaaattcagaagccctgagtgtggcatgaggatgtggaacagctggatctcatccatcgctgtgaagttgtgtagccactccacaaacgtgtggcaaacagccgagccgggagaagggaagacgtgttcaaagattcatatgtggccaggctcagtggctcacgcctgtaatcccagaactttaggggccaaggctgggggatcgcttaagcccaggagtttgagaccagcctaggcaacatagggagaccccatctcaaaaaaaaaaaaaaagaaaaaagaaaagacttcagtgtgcaggtttaccagagttttgtttgcagttgccaaaactgggaagcagcccgcgtgagcccatccacaggtgaatggacagaccgtggtacccgaacactaacagcagccacgggcgtggactgtggtcacacagcagcagggagccgatgagtctcggacatgctaacccagagaggcccattgaggaggacctactgttttttgtgtttttgttttttgttttgaaatggagtctcgctctgtggtgcaggctggagtgcagtggtgtggtcttggctcactgcagcttccgcctcttgggttcaaacagttctcctgcctcagccttccgagtagctgggactacaggcacccgccaccacacccggctaatttttgtattttcagtagagacggcagttcgccatgttggccaggctggtcccaaactcctgaccttgtcatccactcactttggcctcccaaagtgctgaggttgcaggcatgaaccaccgcacccggctggacctactgttttattccatttatgtgacactctattaatagaaaaggcaggggtggggctggtggttatatggtgcacataactgccagaactcagtacacttaaaatgaacatcttaatgtgtgaaatttttttttttgagacggggtcttgctctgtcacccaggctagagtgcagtggtgcgatctccactcactgcaagctctgcctcctgggttcacgccattctcctgcctcagcctcccgagtagctgggactacaggcgcccgccaccacgcctggctaattttttttttttttttgtatttttagtagagacggggtttcacagtgttcgccaggctggtctcgatctcctgacctcgtgatccgcctgcctcggcctccgaaagtgctgggcttgcaggcgtgagccaccatgcccggccaatgtgtgaaaatttaaaagtaccaaagctggaccccaccccagattgctcccatgacactctgtgggtgggacctgggagttgggttttgttttgttttgttttgtttttgagatgaagtctcactctgtcgcctaggctggagtgcagtgacacaatctcggctcacattaacctctgcctcccagatgaaagcgattctcctgcctcagccttctgagtagctgggattacaggcacacaccaccaccccctgctaatttttgtatttttagtagagacggggttttaccatgttggccaggctggtcttgaactcctgacctcgtgatccgcccgcctcggcctcccaaagtgctgggattacaggcgtgagccaccgcgcctggctgggagttgggtttgtaaatctccctgagtggggctggggcagggaactgctgggtctgggtcttcctggctcctctggtctgtggcttcctgactgcggtggccgggggctcccagggcatcgtggccgtctgtcttgctgagcgtggcacgtgcctttccatgctgtggaggagcgtctcccggtatggcgaactgctggttagggtggggcggtgttgccaggtcatccaggtctggcctctgctctcgacatcgccggcgctgttgctcatctgcgcttgtgatgttcgatgcctgctgcacatgtcttggcttccctctttcccggcctctgtgagctccagcgctgcgtcccttctcttcctcctgtagagccgcagagcacacaacattgacctgaaggggacagtcgtgatctttgacgaagctcacaacgtggtgagtctccgctggcctcctaaacacctcctattgcttctggcctttttgtcaagagccacgcaaacctttctggaggggctctggccaaactcctgaagccctaggtgcccaggactggggactgagcacaccaggagcttctgccaccccctcccgccctgatccgatgcctctgctggggctggagactggccagctgggccagggacctgcccgtcaggcgcagggcccccacaggccgctcaccagaccctttccctccagccagctcggggtcagcctgggccagggctgtctcctctgccctcggcagcagcaggcttgtggtcttgcctgcagtgtctctgcccttccggccacatggcttgagactgaggcaggagaatcgcttgaaccttggaggcagaggctgcagtgagccaggatcacaccactgcattccagcctgggtgacaaagcgggattctgtgtcaaaaaaaaaaatgttgactgggcgcgctagctcatgcctataatcccagcactttgggaggctgaggtgggcggatcacgaggtcaagagatcaagaccatcctggccaacatagtgaaacaccgtctctactaaaaatacaaaaaaattagctgggcgtggtggcgtgtgcctatagtcccagctactcaggaggctgaggcaggagaatcactcgaacccaggaggtagaggttgcaatgagccaagatcacaccactgtactccagcctggtgacagagcaagactccgtctcaaaaaaaataaaatcaaaaagaataattggcaattccagtgaaataattgtttgtttgtttgttgagacagggtctccttctgtcgtccaggctggagttcagtggtatgatcttggcccactgcaacctccacctcctgggctcaagccatcctcccacctcagcctcccgagtagccgggactacaggtgcacaccaccacgcccggctaatttttgtattttttgtagaggcggggtttcccagcgttgcccaggctggtcttgaacccctgagctcaagtgatctgcccaccttggcctcccaaagtgctgggattacaggtgtgagccaccgcgcccggcctgaaacaatcgtttctaaatattggtgtgggccacacagtcatgtttggacctacttgtggccttttacagaccccaggccaaggctttgggaacttggctgtcagcctcctgtgccttctgcacccccaccccatttctgctttctggaacccccgatcctgtcctgttctgtggtgattcgggtgtgcttgggctctaggagaagatgtgtgaagaatcggcatcctttgacctgactccccatgacctggcttcaggactggacgtcatagaccaggtgctggaggagcagaccaaggcagcgcagcagggtgagccccacccggagttcagcgcggactcccccagcccaggtgcgttcatagccagactgcttggtcctgaggcctgcgctgctgcagggtgagccccacccggagttcagcacggactcccccagcccaggtgcgttcatagccaggctgcttggtcctgaggcccgtgctactgcagtgggcagcctgccctgtggctgtgtgtggtcggcctgggcaccatctattcaggctggcactgcagggcatccgcttctctcagaggcttcttgggtgtgaattcttcagggtcctgtagcctgtggaagggctggtattgttcagtagttctggtattttccaaagacctatgtcttctcccagccagtatcaacttggcctctactgtgtaaaactggaaaactctactttgtgaagctgagttgggagcatcgcttgaggccaggagtttgagaccagcctgggcaacatggcggaacctcgcccctgccaaaaaattagccaggtgtggtggtgtgctcctgtggtccaagcttttccggaggccgaagtgggaggcgtgcttgagcctgggaggcagagcttccggtgccccagatgactccactgcactccagcctgggcggcagagtgaggccatctcaaaaaaaaaaaaaaggaaaactaaatatattcactgtaagggcattttgcatctttaaatgacccacaaatctggcatgcatcagctgctctgcctgtaggttccttcccagtgtttgtccagaggtgtatttccacacagcgctagtcacggcatacgtggaaaacgtggaaacccttcatggatgttgtcagttggtctatattttctttcttttttttttttttgagatggagtttcacttttgttgcccaggctggagtgcaatggcgcgatcttggctcactgcaacctccgcctcctgggttcaagcaattctcctgcctcagcctcccaagtagctgggatcacaggcgtgcaccaccacgcccagctaattttgtatttttagtagagatggtttctccgtgttggccaggctggtctcgaactcctgacctcacgtgatccacccgcttcggcctcccaaagtgctgggattacaggcgtgagccgccacgcccggcctttgtccatattttctacatggcttctgtaaacagctgactaggagtctgtgtgaatatcttcataggttctgctgtgacactacttgctcgtgagcatctccaggtgtaaacagcatcagcttcccccattttcctttaaaatcgcacatgtggacggacaccacggggaccctggaccctggggagccccgtcctcacccttctcaccaggatggctgcttggtagagagtgagtttgcaaagttggcatttgtttagtacagaagttatcaggtgttctggctttagaatccctttatatatatatatatatacatatatttaagtgacagggtctcactctgttgcccaggctggaatgtggtggtacaatcaaagttccctgtagcctcggcctcctgggctcatgggatcttcccgtctcagcgtcttaaagcgccgggaccacaggtgtgcaccactgccaccggctctcaagattgccacgcagggagttgcagtgggggaaggggttcctgggactttgaacgctccacctccctcctctccacagtcccccaaccccacctctctaacggggtggacggccgcctctttccatccttcgcttggcgcagggtggggagagtgacaggtctccttccctcatctcggcagctgccatttcatcgcttacataacgtgggagaaacatccacccacccccaggcctgtgtgaacatcaccacggggccttctccactcttcagttttgttagttacttgatgtgcagggctttttgttgtaactagtgggggacgtgtggtggggtgggcttctgccatctcattcaggaccagaacttcagttttcatccctatctgttcccccacccctttggagatggggtctcactctgtcacccaggctggagagcggtggtgccatcacggctcactgcagcctccacctcctgcagcctccacctcttgggctcaagtgatcctcctgcctcggcctcccaagctcctgggactacaggcgtgtgccactgtgcttggcagggtccattcttttcctcacactttatttattgaagagcccaggccgtttaccctgcagagtcggaatctgtacaggaggggcagccacacgagttccccggtttactctgaacttaggtggcttgagggccccagttagactgcggccaccgtttgccgggctccagatgggacgtcctttctatcagaaggctcacagtatctcctttcccgtttcttcccatgtgaacattgttgctgctgaacacctgaatatgttaatcactgggggcttgcaagatggcagtgtgctaattccatcatctagtcagttagcaggaataacttaggaccacgccctgcaccatatcagctatgtggtgatcccattcacacaggaaaggtgggacaaatgctgggggtgggccgggtgtgctgtctcacacctgtcatcccagcactttgggaggcccaggcaggcggatcacgaggtcagagattgagaccatcctggccaacacggtgaaaccccgtctctactaaaaatacaaaaaaattagccaggtgtggtggtgcatgcttgtaatcccagctacttgggaggctgaggcaggagaatcacttgaacccaggaggcggaggttgcagtgagccgagatcgcaccattgcactccagcctggcgacagagcgagactccgtctcaaaaatcaatcagtcaatcaagtgtcatcactgaatgtttgtgtgtgaacgtggggattggtcctgccccatgctccctcctgaatctcactcctgacctcagttgctgcaccttgaggtgttttcatgtgggctcttgtgtcctgaccccggcggttgtggcctctttgctgtctgggagtcaggatttttcacactcatgtcctgctccagacctggaatcagccaagtctccaagaagccctgctttcttttcctgcaagacggtatttcaagacccgccatgcggcagcgggttggtcatggttactgggttggtcgttgttactgggtgttttcgtggagatacagccatacgcacaggtgtgttcacaaatgttaattctaaaggtcaaacacccggccaggcataagggctcagcggtaatcccagcactttgggagaccaagactggtggatcacctgaggtcaggagtttaagaccagcctgagcaacagggtgaaaccccatctctactaaaaatgcgaaaattagccgggcatggtggcgcacacctatagtcccagctagtcgggagacagacacgagaattgcttgaacctgggacatggaggttgcagtgagcagagatggcgctgctgcacccctgcctgggtgacagagtgacaccctgtctcaaaaatgaatagataaataaagataaaacacctgctcctcttggtgtctccagtttggatttggcctgtgtagcctcttccttcgcctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccatgttggtggatttggcctgcatggattctgtgtggcctcttcctttccatgttggtgtccttttttccatgccaggaatcctggttctcaagggcggggttgttggcacgagcgtgatgcagactgcctttgctgcctttctcttgcccagggctgaacatggagctggaagacattgcaaagctgaagagtaagtgttgccctccccgcctccttgcagctgggtggggcctcctccttgcgaggaggtgggtgacacctcctcgacccacagtgatcctgctgcgcctggagggggccatcgatgctgttgagctgcctggagacgacagcggtgtcaccaagccagggaggtgagaggcggggagccagccccttcactgcaggcccagcctagagctagaaacgggccatggtgcagtcctgggctgtcacatcacgagtgaggcctgttttcaggcctgttttccctttttgagacctgggaggagcacctgctttgcatgatctggttgctgagatgttgagaggagcagcacacactcccacgggacagcacacagccccccacggaacggcacacacacccatggaacagcacacacactcccacgaacagcacacacactcccacgaacagcacacacactcccacggaacagcacacacacccacggaacggcacacacacccacggaacagcacacacactcccacggaacagcacacacacccacggaacggcacacactcccacggaacggcacactctcccacggaacagcacactctcccacggaacagcacacacactcccacggaacagcacacacacccacggaacggcacacactcccacggaacagcagactctcccacggaacagcacacacactcccacagacagcacacacacacccacggaacagcacactctcccacgcggggccgctgggtttcctgcagtttctcctcctccaggcctttccctggaccctggtccagtccgtcatttgagcacaggtgcctgttagaacgagaccttcttgttaggacgatgagtgtcccagccaccacctcttttggactccgggaggcctggaacgttctgaacgctccgtggggctccagtcttctccgcagccagggcagcagggtttgctgtctgtcctgcaggcagatgaggagtcagggctggggcctgtgtgggggctctcctgagcgcacagccgccgaggtggagcgtgttctgcctgagcgccgacctggtcgggggaatcccagttgcttccaggtggagccactgtcctcagcgtaatgctcaaggctctggcctggctcctcggccaccctgcaccctcagggtcccctcctgtagcttctgctgccccatcactgtcactctccaaagctttggggactctgcccagagccaccgcctcccagaagcccctgacaacctcttgacgaccccctagtgaccccatccctcccctctgacggcggcccctgctctgaggcggcttcttttcctcggtgctgttctcgtgctggccaggcctcctctccccacctggaggctcctgagggcggaggcctctcacctccaatgctggcgtcccctggagggctgaatttgtttccgagggaaggaaacttccacagttgttgccttcagttccaaagctgcagcctgatttccccctccaggctcgagcctgttttcttctcggcagctacatctttgaccagtgtcgtcccccctcaggcccgagcctgccttcttctcctcagttcccaaagctgcagtctggtccccccgccaggctcgagcctgccttcttctcctcggcagctacatctttgagctgtttgctgaagcccagatcacgtttcagaccaagggctgcatcctggactcgctggaccagatcatccagcacctggcaggacgtgagtgctggcacggggtctttggtgcgggcaaatgtggcgtagggggtgcagcaggcctccatcttggcagtcagggctcccctggccgtcacctggccgtcagcaggaacaggcccacagaacctcatcttctgatcggggcgtggaggcgttagtgccacttgccagctgccgtagagcctgtcccagttctgcagctggcggcttcgtcctacagcctcatcccattattctgcttttgagaaagagcagcccaaggccctagctggcttgtggggcctctggcttctccacaccaccccgagttctgcttctcagagttgtggggtccagaggctttgcccagaggcggtgtccccatgggctgctctggtttgagacgccgggcccagcggggtctctcctctgctgcgctcccgggtgctggggagggtggcttttgctgcttcaacccttaggcgaccatagagcctcttttcaagtcccactgacccccttggagactctgtccctgcctggcttctctcctggctgctgggaagagcaggcgaactgcccgccctgaatggatgctgcgctccaccctgggccccccattgggcaggagatggagcttggcagtcgggctgagcgggctcatgctggaagggccggggctggggtcggggcctcccctgcctgcagtgtgggtgtcagcgccctgctgccctccaggtgctggagtgttcaccaacacggccggactgcagaagctggcggacattatccaggtggggcctgctcctctgtggcatctccttccctgatggaagccgggcgggtgccttctcctgctgtattagttaactgattctagacttggggatgggagaaaggcccctacaccacctgtttctgattggcaaactctcggctcctttccagtgccctaaacccacactgggcctcctgcagggatgggggaggacgaggtctggtggcacatgcccagggtgatgctggtgagggaggacgcaaaggacagtgggggccggggagccgctcctgccctgtccgggccctcaggccaggggggacccactgctggcagccccagcagccccagctgcacgcagatgaagagctctggacacacgcggcttcctgaacagcttctccagggacagacaaatggggaccctgcaggttcccggcaggggtgtccctgggagcccatgattgggggtgcgaccctggcccccttctcattggccccgtcctgtcctgcaatgcccgtcccatgtgaggtctgcttctggctccatgcctatggcagcacctgctttccctggcgtagaggtgcttgtccggtttgtggagggcacgccccattttgggtgctctgggcacgttgcctctccggggcctcggtggcttttttagaagcagactcagaagtccctgactggggaagccaaggcacaggtggctgtgtggagccctgtgaggcctcctctgtgctgcccacgctgtacctgctggccacacgagatcatggcagggttaggcagggctgcccagcgctatgacagcttcatgagtgtccatctggcctgtggggtgcttgagctgggggaggccgcagaagaaccctgggatgcatggctggcctgtgcatgctgctgggcatggagctgcagatcccggaacaagcaggcactgccttctccttcacagacgcagctctgagcgggggcgagacctgggcagggaccaggtggggtgggcacagggtggtggggcccaggctcagccctccctccactgtggccgtctctgtggccagtgacgccacagcctgtgtcttctctgtgcggtagctggggctggaaggacagcactgccttgtcctcccaactcctccccaaaggcacggtgggcatcccaggcccagacccctctgtctgtggctcctgcctgccaagggctgctgtgctgtcccgcatggagtgtggttggctcttcaagcaggaggccgtgcacctatcaggcggacctgcttccatgtccctgatgggtcactgcaaagcacctccagcacatggccaggcgaggtagccctgcagcccagggcctggagggcaggtgtgagctggcccgggcctgtccctccctggaatacagcttcccaggctcccacttatggagaagtctcctccacactatggaactgaatcctagaatgtggcttctgaggttcctacactcgaactgaatcctggaatgcggcttccaaggcttccagctatggagaagactccacactctggaaccgaatcctggaacgcggcctcccaggcccccagctatggagaagactccacactctggaaccgaatcctggaacgcggcctcccaggcccccagctatggagaagactccacactctggaaccggatcctggaacgcggcctcccagcctcccacttaaggagaagtctccacactctggaaccggatcctggaacgtggcctcccaggcccccacttaaggagaagactccacactctggaaccgaatcctgcacactccatcggtttggaatttcctttggctgctgctctaagtagccgctggtggatgactcagcttctgccagccctcgggtgcctggaggatgagggactgcacacagtgctcacccgcgttggctcctgagcccctgcaggtgtgggcggtgcccatagggctggtgctgggttgggcctgcagccctgagtcacaggtgaccctgggggcagagtggggccagtggccccaggaagaggatgtgggatgcacagctcagctggaggcgaactccaggcagggtcaggccgtgtgctcggaagtcagggcttagctggaggcaaactctgggcagtgctggcccgtgttggggaaccagttgcccctgggcccccgtgagactgctgggtcctcatccctctctgcctgaggccggagctgccctgggctgaggcacagggggatttgtggtggtgtttttttgagaaagggtctcgctttgtcaccccggctggagtgcaggggcttgatcacagctcactgcagcctcaacctcctgggcccaagtgatcctcttgcctcagccacccgaggagctgtgaacacaggtgtgcaccaccgcactcagctaatttttaaaatttttttgtagagatgaggtcttgccatgtttcccaggctggtctcaaactcctgggctcaggcagtctgcccgccttggcctcccaaagtgctgggattacaggcaagagcttccatgcctgcccagcagaaggcttttcgaaggaagctgtttcctgaggcagactcagccctgctcatggcagccaccagcgtgggggtgaacttgttctgttacttccatccccgtgggccaaatgctttggtaaaacacaaggccctgtgtttagctgtcttgacagtgaaaatggctgggaaggaaggaaggaacggaaggaaatttctctctccttctgtgcgtacccaggcacgtgcacatgcatgcagagtacgcacacacgcacgcacgcctgcacaaatccacgcatgttgccaagtctctgtgttccagccgtggtgtctgccccccggtgttctctagttcggcttctccgcatttctgtgaatgattccggcttcttggtgttcccagcagaactccctcaagtctgcggcggggctctgacggcggtggcttggctgacatggccacattgctgagcctgttgggggctttgcgttcctgttctggccgtttttggctcgttttccaggaacggtcgtcacgcgctcctctcctagtgcaggcatcattcctttcccattgatttgcagggttctctgtaagttctgaggatcccatatacatatactctctgtaagttctgaggatcccatatacatattctctctctaagttctgaggatcccatatacatattctctctctaagttctgaggatcccatgccgacatacatattctttccttgtctcatgctggtcattttttccattttcatgacaggtttggtgaacacatgtttccttgtcagatttttgttctgagcttgtgcctcccgaccaagatgctaaaccgggtcttgtgtattctccaaactgcactgtagagtgacggagctttgtgtctgggcctccatgccttctgacgtcacctgtgggggtgtgaaaggcagactctaccttgatttttcccagcacgccacaccggtggttctgtgcgctgaccgagcggctcggcttcccccaactccactgggcacctgccacacttttcctcatgtttttgttcactgtggttttgtcgtaagtcctggtgttggcctgaaccaatttctttttgtttgtttttgagacagagttttgctcttgttgcccaggctggagtgcagtggcgcgatctcggctcactgcaagctccgcctcccgggttcacgccattctcctgcctcagcctcccaaatacctgggattataggcacctgccaccacgcctggctaattttttgtatttttagtagagacgaggtttcaccgtgttagccaggatggtctcgatctcctgacctcgtgatccgcctcccaaagtgctgggattacaggcatgagccaccgtgcccagcctgatatttttagtagaaatggggttttgccatgttggccaggctggtctcgaactcctgacctcaggtgatcctctcaccttggcctcccagagtgctgggattacgggtgtgagccaccacgcccggcctcttgttcttttgaaacctgccctgacgttttttccatagtgcatcttggagtcagcgtgtctacttcctgtaaaaatcttactgtgattttgactagaatgtgttgaattcctgttttttttttgagtcagggtctctctgttgcccaggctggagtgcagtgggaccatcacagctcactgcagcctcaacctcctgggctcaggggatcctctcagctcaacctcccaagtagctgggaccacaggcacatgccaccatgcccggctaggtttttttttttttttttttggtgaacaccctggggttgcaccatgttgcccaggctggtctcgaactcctgggttcgggcagtttgctcctctcagcctcccggagtgctgggattacaggcctgagccactgcactaggccatgttgaatttctagattaatttggggccctcaggggcacagagaggagggctgggccagttggcgggaggagaggcccctcgggctgccgcattttcagtgcatggagatggcctatgttgggggaacacagagctcaccgggggtccctgcagggaggagaaagggtcaggcaggtgccagctcctgtccattggcctggggctgcatgatggcaggggccggtgaaccgatgacccctgggtgtcctgtgaccttctgtgtatgcggctgatgctgcagaaagtcgggtggcctcaggctcctgacggggctgcacttcctctgcctttcagattgtgttcagtgtggacccctccgagggcagccctggttccccagcagggctgggggccttacagtcctataaggtaggggccacctccaggaggcaggtggagggcagcccttgttccccggcagggctgggggccttacagtcctataaggtgggggccacctccaggaggcaggtggggctgggggtcttctggtcctaaaaggtaaggggctgcccccaggacatgggcggggcctccacactcctggtcctgtcccctccaggtgcacatccatcctgatgctggtcaccggaggacggctcagcggtctgatgcctggagcaccactgcagccagaaagcgaggtacagacctgggcccacacgctccccgcccgcccgggtgcagtgcccggcaccaccatgccacaggctaggcacatgcccagccgtggatctcctgcccccatgggcctggccaccttctccatatccaggccaatccagagcattctcctcactgtccctctgaagattggagttactgagagacgtaggagatggcctgatggcaccgtgacctgcccagagtcacctggttggtggtggcagagccacagcccagccaggcctccctgctgggacacgctcgtttatgccgaggccgtcagcacagagcctccacagtgaggcacggctctgcctgctgcctccacgcagcgcctggccgggccaagcctcagggtcacatctgaagggggcccggctggccctgttgtccgaagcccctggtgcgctcagccccgaggccccacgtgccttcttggcttcctgtgctccgtggcgtcttcgagtcggtgctgccggggacgctgtgtggatggggtctgtgagtgtgccctcggctccgtgtccggagccctgtggttcttggggtgtatctggccccacccccactgcgtggtgtccagggtggggcttcacggctgcagctgcgggagctgctgcccctgccttgtgctccagtggggccttgcctctgggcttggttcgtccctctctggaacattctttctcagctgctgtccgacccatggtggcatgacgtggccctggctgaagcagcccttgtgcggttgctgtggttgggtctgcctggccgagccggaagggaagggctgggagggcgtcagggtggcgtggcttgacccccgctcggtgatggtcctgcagcaaggcctctcccagcaggaagcgtccatcccggggggaggccggcgcccctcacgcagttggggttgcgggaggcagtgcgtgcctgaggcagccggtgcacagattccaagggcctggaatctgtttgttccattgacctctgatgtcacttgacttctcagaagcagccactccctgcactgggcgtttgtaggaaatgagctcctggaggagggggtggggaagttcccccattgcagggcacactcagccccaggaaggaaacgtgcctcgtccctgctgactccgaatcgcagtcagagtcgttctgcttgtgccgtgttgaattcccggcatccggcatccagactcagcctcctccccaggccacggccgccgtggccagtcggtcaagcccttctaggaacttcctttgagctggcgcccttgttcactgctgacgccactcagaggcttgtgcacgtgtcctgcttccaggcagagctgggaactcgcaccccgtcttctgcacgcggccgtggaatgtcgggatgccggcgcttccttcccgtgtgctcttggcggggtgggcttcttgccctgagccgcatgtcacagtttctgcagaagtttagggttggagtgggctgacctctctgcaggtgtccccagcctctgcctggggtctgcctcctactcccaggaccccctgtcccccagaggggccccaagctggcaggctcacactcagggcagcctcctttgttctgacttctgcacagtgggcctgggtggctgcccgcggctcgcttgcttgatgccagtgggtggagagggtgatgggcagagaggcaggtggtcaggcccccagtcccgtcctcacactctgtgccctctgccgccccccgccccacagggaaggtgctgagctactggtgcttcagtcccggccacagcatgcacgagctggtccgccagggcgtccgctccctcatccttaccagcggcacgctggccccggtgtcctcctttgctctggagatgcagatgtacgggccacccctgccagggcctgagcaccggtgacacctctgacatcagcggggtggaagtggtgggggtccccatgagccgggtgctgggggtctcgggcctcgagggctaaaggggtgctggtgcacttccccactgtctgctccctctggccacgctcagccctttcccagtctgcctggagaacccacacatcatcgacaagcaccagatctgggtgggggtcgtccccagaggccccgatggagcccagttgagctccgcgtttgacagacggtgagggcctgtccctgggccctgctggggtgggaggtgggggagcactgaggcctgaggtcctgagcagtggcctctccggctctaggttttccgaggagtgcttatcctccctggggaaggctctgggtgagtgccctgaatgccccagctgtgcccatcctggatcctggacccctgctcccaagagctggtagggaaccctgcagacatcctgcccctgccttgaccccggcccctgcacttccaggcaacatcgcccgcgtggtgccctatgggctcctgatcttcttcccttcctatcctgtcatggagaagagcctggagttctggcgggtgcgtctcccctgtgttctgggcggggtgggtgagggcagggctggagcatgaagcaggcagtggtcacagctcctgcttgccctcatcggatcggcggcgtgaccagggctgccgtgtccctgcctcttcctcccacaggcccgcgacttggccaggaagatggaggcgctgaagccgctgtttgtggagcccaggagcaaaggcagcttctccgaggtcggcacttggccggggctctgggcctgctgccccctcgtgcctcccctgcctctcacagcttccccaaggctgaccactggccctgaccatgggctccggcggctcccgctgcctcttcagggctcctgcgtttccttcctggccctgagtgttgcctcttatcttacaaagcccccagcaccgggtgggtgtggtaacagtggccctcctgtctgagtagccctagtcggccaccctggccctggggttccccgtgttttctgggaagcactgagcaggcgtggggtcagcctgggatccgtgccaggaagaagcttccagaacccgattggccttcctggctaggacgatccttcatcttggagcatgagacctgggtctccctcatgggggaggaaggggctgggggggggctccaggctcagcctcaccaactttccttccagaccatcagtgcttactatgcaagggttgccgcccctgggtccaccggcgccaccttcctggcggtctgccggggcaaggtgagctctccagggccctctgccctgacctggttgcctgttccctggtgggtgcttatggctccccagcagactctgggccctgggggctgcccggtcccctccttgggtcccacgagagcgactgctggccctgctgggagcgtgtcctgctctgggcctgggcaggcaggatgggagtttcctggccacaagagttggaggtggcgtctgggagctgtggaccccaagtggggtcctgacccacagatggagcttcctcccacccctggttggggacggagcctcggggaaggtggctgggctgggtgtgggcaccagggagaggagcccccacggccccaggcagctccctggtgtgtcccctaggccagcgaggggctggacttctcagacacgaatggccgtggtgtgattgtcacgggcctcccgtaccccccacgcatggacccccgggttgtcctcaagatgcagttcctggatgagatgaagggccagggtggggctgggggccaggtgagttacagcagggtggggctggggtaaggcggtctggtgactgagcccccgccccgtggccaagggagcccccgtgaccgagccgcctcgccccacagttcctctctgggcaggagtggtaccggcagcaggcgtccagggctgtgaaccaggccatcgggcgagtgatccggcaccgccaggactacggagctgtcttcctctgtgaccacaggtgcgtgcagtccggtggcaggcgcggcgccaggggacacgcccacaccccactgggcccctggactctccttccccacatgaggccccgtctcctccagagcctctccggctactcggggtcagcgtggggcccctgcagcagatgagggtcttcacttcggtgaactgaacccttgaagcggctgtgggcagggcagcagggctatggccaccccccaggttcgcctttgccgacgcaagagcccaactgccctcctgggtgcgtccccacgtcagggtgtatgacaactttggccatgtcatccgagacgtggcccagttcttccgtgttgccgagcgaactgtgagttcctgcccagggaggggatgagggtgttgtccccagaggagccagaaatgggtccacccacccccatggttctgcagatgccagcgccggccccccgggctacagcacccagtgtgcgtggagaagatgctgtcagcgaggccaagtcgcctggccccttcttctccaccaggaaagctaagagtctggacctgcatgtccccagcctgaagcagaggtcctcaggtgcggacgggcagcgctgggtgggcggtgtgggggtggcggagcgggcggcgtggggcgggcagcaccaggcgcccagggcggaggcgactcacctggctttgtgcgcttcccctcccacctccaaaggctgcctctccctcctagggcagggcccccacgggctgcaaccctcccctacaggcagagaacgccccaggcaaggatgccccccgaggctgagactccccccaatagcagggaggacacccacaggcaggaccccaagtgctgggactctcccccaagaggggctttgccacaggcagggaccccagctggggccccccgtgggcttcactgcgcactcgggtgcccctgcagggtcaccagctgccggggaccccgagagtagcctgtgtgtggagtatgagcaggagccagttcctgcccggcagaggcccagggggctgctggccgccctggagcacagcgaacagcgggcggggagccctggcgaggagcaggtacagttccagggccttgggatggacacagaccctctgtctcctgaggccaacccgaccccgcccatctggcctcaggcacctccccacacacccctgtaaatcccctgcctggcaggcaggcgggcaagcgggcgggggatcccagctgcctggctgtctgtgggtcctccaccccacctcacccacaggctgctggctcccaggtggtgcatgccctggccctccgcgggtgccccccacatcactttggttctctggcgggtcagcttggctcagtgcactcaaggtcgggtgcccctgccactggctgcgcttgaggctggcctttctccaggaatgtgctgcgggtggaacccaggttccttcttccttggggccttttgccccagaagcccataattcctcaggccaacccgaaattttctccctgcttcctgctgggagccattcccctcttcctgcccatccctgcccttcaggcccctggagtgagctccaggtgcaggcaccaggcacctgtgtccccttcctgccagcccctcgctgtggtcggactgtcttccctggacctgctcttacaagtcaccacctgcgagcctcatgagccgctggtgtgacttggacaggaccaagttgtggcactgtcaccggggtgtgctgtgcccccctcccccgacctccatcttggctcagggctccttgggaccatcttccctgtgcgtccaggtgctttgggaccccagagtgtgtggttggggtctgtgtgtggttgtgagctgtgtcctcctcaggcccacagctgctccaccctgtccctcctgtctgagaagaggccggcagaagaaccgcgaggagggaggaagaagatccggctggtcagccacccggtgcgtgagctgtccctgcacctgtgccgaccaccatagacacgcatgggaacgcagccgtgggtgcccccagccacggctggtcccgatgggaccagggaatccacccccaggagctgatgtccagggcagctgtgatgctgacggccaggggctcaagtgtgtggtttcttctgcagggggctcatgagtcccagctggaatcaggccccacccttgggcaggtttggcatggggcctgcagcactgggcttggccctggcatttccctcaagtgtggatgcacacctgcctcatgtgagggacacagcccattcctagccttggatcaaagaacggagttatagccggagccaggaagccccctgcctgctggaaaaccccaagtgtggcggcctttgtccatgtcccttggcttctgggaagaactgggtggtgcccaggcagggctggtgccatcaggaagtgggtggctgctgaggggcctgggctggcgagggcctgggtggggagtgcctgggccgcccctgccttggtttccacgtttccgtgttggtctggggtgtgtagagagatgggcactgctcatccggaagcccctccttgtgcgctgccatcctgggagcctcagccgcatccgctgtggggcagggggcttgagggaggaggagagagacgggccatgcaggacccctggcttgaggcagagccaatctaccctttgcccattcactgctctcagttccctgccagcctctcactgtgtgacctcagacgggcccagccccacagctttcttcccgcagcccctccctatgtccatccagccagccagtttctcaggcagcagccccacctcggcagtcactgtcccagggaacgctcaatgttccaaggaaggctctgcagccccagggaccagatgatgaggctggccctgatggagcctcgggcctgtgtcctgcaggaggagcccgtggctggtgcacagacggacagggccaagctcttcatggtggccgtgaagcaggagttgagccaagccaactttgccaccttcacccaggccctgcaggactacaagggttccgatgacttcgccgccctggccgcctgtctcggccccctctttgctgaggaccccaagaagcacaacctgctccaaggtgccctggcttgcagaggccacccaccctgagggcagtgctgccgccgcgtgtggggtgggggccatctgggtccaaggtggtctctgttctctagagaaaaaggggcagatggggacagacgccccttcctctacaggcttctaccagtttgtgcggccccaccataagcagcagtttgaggaggtctgtatccagctgacaggacgaggctgtggctatcggcctgagcacagcattccccgaaggcagcgggcacagccggtcctggaccccactggtaaatggggccccaggtgggaccctcagactcctgcgtggaaggcagtgtgggccagagtcctgggctgcttggggtgggcatcctcgggccctgcttggccccgcctctctgttcccctatgggagtgatgggggcctccacctccaccaccagcaccagcagcaccacctccacctccacctccacctccacctccaccaccacctccacctccaccaccacctcctccaccaccaccacctccaccaccaccaccaccaccaccacctccacctccaccacctccacctccaccaccaccacctccacctccaccaccaccacctccacctccaccaccacctccaccaccaccacctccacctccaccaccacctccaccaccaccaccaccaccaccaccaccaccaccaccacctccaccaccaccacctgcaccaccacctccacctccaccaccaccaccacctccacctccaccagcagcagcatcacttgttggggagaccctgtgcaactccatgcacagccctgtccctgccatagccccgacccctaagcacagccctgtccaactgccacacgtcccctgcctcccatgcatggtcctggggggtcaactgcacacgccagggtcctagggtcctagacccctgtcctccctgtttctgcctctgtttggggtggagtccaagtctccagaggcggaagcatctgtgttcgtgtgttaatgaacagcccctacagagttcccctagttcacccaggggggaacctagcctgttgggacgaccccagatcccttctgggcttggtactcactgggatatcctcatgcctgcacccagcctacggctctgagctcctgagtggggctttggcctgcccgccactgttccagcccccatccagcaggctggtgtctcctctgatgcccccagcacccaggcgtgtacctgcctgggttttcccgccctggtctgaggtgggtgaggcctggcctccctagccagccctgcccccccaccccagggaactttccagatgctctcgaccagctttgtggctctacatctcttcatcaggaagaacggcgccggatcccaagctgaccgtgtccacggctgcagcccagcagctggacccccaagagcacctgaaccagggcaggccccacctgtcgcccaggccacccccaacaggtagctgactcctgaaccgtgtgcagcctacgacttggtgggtccctcagtggcttcacgaggctaactcttgagtgtggccggggctgcccctgtggggagccatctcatggtggggactgctcccggttctgcaccccgcagttgtcctgagcagctctccaggagttcctggaggaagggcgggcagggcggtgggactctcagtcctccaccccagcgccactctgagccatgctactcccacaccaggagaccctggcagccaaccacagtgggggtctggagtgcccagagcagggaagcagggccagcacgccgtgagcgcctacctggctgatgcccgcagggccctggggtccgcgggctgtagccaactcttggcagcgctgacagcctataagcaagacgacgacctcgacaaggtgctggctgtgttggccgccctgaccactgcaaagccagaggacttccccctgctgcacagcaagtggccctggcgtggggaacagccggtggggtggggggcaggggacaaaatgggggctgtgccgggtctgattgaagctccccgcagggttcagcatgtttgtgcgtccacaccacaagcagcgcttctcacagacgtgcacagacctgaccggccggccctacccgggcatggagccaccgggaccccaggaggagaggcttgccgtgcctcctgtgcttacccacagggctccccaaccaggtagggcacctgcctggctgctcctggcagcgccccaaccgcacgcagccctgggagtgagcagcaaagccccaggcccccctcagactcaagtctctgtctccaggcccctcacggtccgagaagaccgggaagacccagagcaagatctcgtccttccttagacagaggccagcagggactgtgggggcgggcggtgaggatgcaggtcccagccagtcctcaggacctccccacgggcctgcagcatctgagtggggtgagcctcatgggagagacatcgctgggcagcaggccacgggagctccgggcgggcccctctcagcaggctgtgtgtgccagggctgtggggcagaggacgtggtgcccttccagtgccctgcctgtgacttccagcgctgccaagcctgctggcaacggcaccttcaggttggtgcctggccactacagttcctgctgggtgtagccccaggtgatgggctgagggggaaagggcaggcccttgtcctggtggcaacgcctggcagacgtgtgcagtgggccggttgtctcacaggcctctaggatgtgcccagcctgccacaccgcctccaggaagcagagcgtcatgcaggtcttctggccagagccccagtgagtgcccacggaggcccccagcacacccaacgtggcttgatcacctgcctgtccagctctggtgggccaagaacccacccaacagaataggccagcccatgccagccggcttggcccgctgcaggcctcaggcaggcggggcccatggttggtccctgcggtgggaccggatctgggcctgcctctgagaagccctgagctaccttggggtctggggtgggtttctgggaaagtgcttccccagaacttccctggctcctggcctgtgagtggtgccacaggggcaccccagctgagcccctcaccgggaaggaggagacccccgtgggcacgtgtccacttttaatcaggggacagggctctctaataaagctgctggcagtgcccaggacggtg

SEQ ID NO: 2 - DNA, 37214 bp, Macaca fascicularis
  misc_feature 4525..4624: note = n is a, c, g, or t
  misc_feature 4914..5013: note = n is a, c, g, or t
  misc_feature 9472..9571: note = n is a, c, g, or t
  misc_feature 16613..17049: note = n is a, c, g, or t
  misc_feature 19812..19911: note = n is a, c, g, or t
  misc_feature 35806..36357: note = n is a, c, g, or t
  source 1..37214: mol_type = genomic DNA
atgcccaagatagtcctgaatggtgtgaccatagacttcccattccagccctacaaatgccaacaggagtacatgaccaaggtcctggaatgcctgcagcaggtagagcacaggctgggaggaaaggactgcaggcaggtggagctggggctgggtgtgcgtccctctcccgacccattccagccaggcccctccagaggcagcctctgtcataaaaaggctggtgttccaggtggggtcagagagaggattgacaagtaaaaacatcgtccttgagagtgacagacttagtgccttgggggcctctgaggagcggctggtattcacaggtcaggaggaagcatttccatcagaggggcagccgggggccagcctcactttgaaggtctttgaacctttggggtgcagggaggtggcagcggcgcaggttgccttctcctgggttccttgaggtgccctcttgtacccggctcacacccctcccctccccgagtttcccgctcaggttcccatctgagagctcgtatgtaggacatcagataggacagcgtaagtgtttggatctggaaacacagaacagtttcctattttgagacttgatgcctaattagtcatcttactatttaggctgaaaaatactgttgcatttcgggtaacgttctgcaaatggtctgctgatggcagctggagttgcttcacgccctttagggcaagagtggaaagtgtcctgcggacttatccatggtcccgtggggctctcgccacctgacagcggcctctgcatctgcgaagagctgcccactggctgctgaggcttgtctcagggtggtttgtgtggccttgcctcttcctggcttccccgtaacccttgcccccaactctgttcagaaggtgaacggcattctggagagccccacgggcacggggaagacgctgtgcctgctgtgtaccacgctggcctggcgagaacacctccgagacaccatctctgcccgcaagattgccgagagggttcaaggggagcttttcccgaatcaggccttgtcgtcctggggcaacgctgctgctgctgctgaagatcccataggtgaccctagttcccaggccttccctgacctcctgtggggatggctggcaagggatggcgctgagggtagggtgggcccatggggacttctgccagcagagctcaaggagaattttgtagctgccacataatttctcgccatcgtgggtataaacctagggttgggcttttttgctgaattagggcgtggcaggtgtacacttcgcccgtttgtgataaacgagtctctggggtgtcagattcttggctgtctgcagggctgagttagccgaatgccacccgcctttaacacgtgagaaccatgacctctgtgcttgcttgtgtctgggcagcttgctacacggatatcccaaagatcatttacgcctccaggacccactcgcaactcacgcaggtcatcaacgagcttcggaacacctcctaccggtgggtcagacgagttcacgcctgtcttggggtcctcgagagaaccagcttgacgtggtgctgggtccacagaccacgctgtgctgtagtggagggggcggtctttccagacgctccccagaagtatgcagtgtgctggtgcccaggggtggggcggggcccgggctgtccctagtgcccattacttgtgaggaggcagctttgcatttgtgtgctgaccttgggctggcgtcctgagctcctttcaggtgctgttgtggcagctgtgtggcaggtcagggccggcccccagtgcagctttgcacatgaagtgggaggaggccctgccgcttgtcagagccctgcagagtcttggtgttctgtcgggttcctgtggaggggccgatggcggggtgctgtggaagctgtcgaatctcgtccctctgtccagttcccccgcttgtctccttgctccctcctactcccgggccacgatcctatgccggggccactctcctgcacccggaccacgctcctacgcccaggccactctcctacgcctggaccacattgcagttatgctgacttcctctggctgccagtgctcctgtgtgtctccatacagctcacgctgcgggaccacgctgtggctgttggaggcagctcctcctccacccatagtgctctctccctccaggcctaaggtgtgtgtgctgggctcccgggagcagctgtgcatccatccagaggtgaagaaacaagagagtaaccacatgcaggtgggctcctggctcccgctccggctcagggttccgtgaggcgagtgctgctgggtatccagagtcccaggctgtgctcctgctgggctggggttcgaagttcactgggggactgcaggcgagggcctggtgggggtggggactggctcgggtcctttcttggccatactccagccgcgcactctgcccttcctcccgcagatccacttgtgccgcaagaaggtggcaagtcgctcctgtcatttctacaacaacgtggaaggtacaggcagctcggtgggaccaggcttgggttggagtgtgtgcagcctctcagggcgtcagcctggttgtgcttgccgggtggggcggccagtgcagccgtgtacctgggcactgtcttctgactcgggaccacccgtgttagtcttctgtgtggaagagctcacccagtggtctgagacagccagccggccggactgcctgtggctggtgcctggggccttggattttgggaaggctccctccatttccggacgagaagggctccctgcacctgactactggtgcatacagcagcggttttgctgaacacctgctttgtcttcggggagcctgaggttttgttgcttgcccagcagcgggtgctcctgtgaccggcccttgtaccatcttgggagggtgtcctggaagccgtgtctggcctcccccgaccttgccgcatgtgtctttgtcctgtgctgacgttgcagagaaaaattacagccccgagcgtgactccaggctgagtcctgtgggtccgacacgggaggccttggggcctcttcaggagacgggatgagtgagtgatgggagcagagccagggcacctcgccctgtgactgcaggtggccacagcctgtgagggccaggggtgcttctccacccacgtggctgcccctcgggtatgtcaggggcttctggggctcttcacgaggtctcagagacagtagcagggtgtgcccccgtcggctgcccttacagtttctgtgacctgagggtggcatctgtgcggttggcatggcctgagcttctgtgggatcagagttccctttgtttcctgcctcagttggggctcaagcctcaggtgacgtggccctggagcacctggaaggcgtcggcggtcctgcgggctgctgtctgcactcgtgtttgctgagtgctcagtatgccaggactgaggaccctgaagctgctgttatatttaggacggttctcccctggcagagacggagccgggtggtcccgcctgacccaccaccaggcgtttctgggccctggagggacagggtgggcggaacatgggcctgcagggagacctccacttactggaggctcgtgctgtgttgctggaggcatcttctgtgttgcttcttgtttgctgtgtcttttgttctggtggcaccaaggacctccagtcaccttgatgtgtggttgtccaggcctttttggtggtcctgagaaggggtctgaggggctctgcctctgccgccaggttccctgatggaagctgcccactcgtcctggtggtgcccacaggacgtgactctggcacaggagggcagatgtgctgttggagattctcagcccgtcgtcgtcatttccaaagtcctcacccgcatttctgggacaggacaggagtgcctgctgggtgtccccggtcccatgcagcgggggtccttgggatagtgtggaacgctgagtgtgggcctgtccggcggtggtcctggacaagggtagcacccctgtggccgccgggcctggtgcctggtgcctagcggggaggccgctgaccgcctcctgtgctgcttccatccgtgtcaggcctctgggtgttgggcccccatctgtctccccccaggcctgtgggatcagtccaagaagactcaatccagccccagcctgtcccccttggcttgggctctcactgcccgacctggcaggaggttgcctagcggtgaacctttgcatcctgtctgccccctggacaggctctgagggggtgtctgcaacacctgtgccagcctgggcatctttttttttttttttttcttttgagacgaaatctcgctctgccgcccaggctggagtgcagtggcgtgatctcagctcactgcaagctccacctcccgggttcacgccattctcctgcctcagcctcccgagtagttgggactacaggtgcccgccaccatgcccggctaattttttgtattttttttctttttttttttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttgagacggagtctcgctatgtcgcccagggtggagtgcagtggccggatctcagctcactgcaagctccgcctcccgggtttttacgccattctcctgcctcagcctcccgagtagccgggactacaggcgcccgccacctcgcccggctagttttttgtattttttagtagagacggggtttcactgtgttagccaggatggtctcgaactcctgacctcgtgatccgcccgtctcggcctcccaaagtgctgggattacaggcttgagccaccgcgcccggccttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttgtatatttttttagtagagatggggtttcaccgtgttagccaggatggtctcgatgatctgaccgcgtgatccgcccacctcggcctcccaaagtgctgggattgcaggcttgagccaccgtgcccggccagcctgggcatctttagagtgggctgcagctcctggaggggtctgagaggaaggaaggcaggtattttgtgaatgaagagacagctagagagctggcacccttcctggcctgcgtcctgtgaggactctggttggggacagcaagcttggggttagcctggggcagagcctctgggaggcccctgccccttgtcccccttccccttgcagctcctgtcctcgccccgccctcagctctctgccagggaaggtttggcaagtgccgctgtgcggcagtgcgtgctgattggctggtcgttgctatggtgccgctcagggatatgcttttcctcccctgccttccctgctatccctgggagcatctggagttgagtcatcgctggtgtgtgtgtgtgtgtgtgagtgtgtgtgtgagtgtgtgagtgtgtgtgtgcgtgtgtgtgtgtgtgtgtgagtgtgtgagtgtgtgtgtgtgagtgtgtgtgtgagtgtgtgtgtgtgtgagtgtgtgtgtgtgtgtgtgtgtgtgtgtgtgtgagtgtgtgtgtgagtgtgtgtgtgtgtgtgtgtgtgtgagtgtgtgtgtgtgtgtgtgtgtcagtgtgtgtgtgtgtgtgtgtgtgtgtgtgagtgtgtgtgtgtgtgtgtgtgtgtgagtgtgtgtgtgagtgtgtgtgtgagtgtgtgtgtgagtgtgtgtgagtgtgtgtgagtgtgtgtgtgagtgtgtgtgagagtgtgtgtgagagtgtgtgtgagtgtgtgtgtgtgagtgtgtgtgtgagtgtgtgtgtgagagtgtgtgtgtgagtgtgtgtgtgtgtgtgagagtgtgtgtgtgtgagtgtgtgtgtgtgagtgtgtgtgtgtgtgtttgtgcccgtgtgcgcctctggcctctgcagctgagtcctggctctcagggggcctggcacctcccggggatgggcacaaagcagccacgatgcagccgggagctggggaaggccccattgccccacacggctgccctaagactctggggtgcttcttggaagaggtccttgcttctgtctgtgtttaagcagctccctaaggagttcttgtggttccaggttgggggcctgtgctgtggaggcgagggaggggcaggacccccaactccctgaagccttttctggcctggtcctctcagccagcataggcagggccctttcccagggctgccatgctgcagaggggagcgggccactatgtagcctaggaaaacctgacgttccttccgctggcagttctgggcctgttgtggttggcagggaagctgagccacggtgctcatcacaggggcacctccaggatttttgggagatgcctccgtgggctggggcgatacgctgaggagctgttcttccctgccctgaggagggctgagtgtagccgtcacccctgtcctgtcttgggctgtctcagggaggatgcatagaaccctcggaaccctgctggcccttgtctgatccaccctcaacctcaggccttctaggggcacaggagggctccgtcgggatcactgcgggtgggggcctcccttggacacctgagaccctcaatgggtgctttgtggcacgttcatgattggggcggggggcacccagccctgcctgccgtgtaggagctgttctgtcctgggcgtccctctgtggttgggacccggagggtgtgtgtttaccccgcctcacacctgcagagaaaggcctggagcaggagttggccagccccatcctggacatcgaggacttggtcaagagcggaagcaagcacaggtgagacccctcagtgaggccacgaccactgtccttccatggcccagctctcctgtgacctgtggaggcccagatatgtttcttcccttttctttgttcctttctaagtcgtgaaactaaccaccgtttagtgtgagaaagttgaagcagctctgaggaacgtagagtaaaaagcgaaaccgcctccctcttccctggccgtcagccaccccaggagccaccgtggagcggggacagagccctgcaggctcgtgtctgtgtggaagcacgcacggttttgtctgcacagactgttctgcagtttgccgttttcactcagcgttgtgggtagcttcgcatgctggtgctggtcgtccggcttcgttcttctgaggacagcaaatatgtcctttgtccacctcttttatagtttcagagattcaagttataataaagcttttcttatattgagggggagagctcctgcccctctttttttttttagacagagtctcgctctgctgtccaggctggagtgcagcgtcgtgatcttggctcactgcagtctcaacctcccaggctcaagtgattctctcagctcagcctcccaagtagctgggactgcaggcatgcaccaccacgcctggttaatttttgtattttttgtacggacaaggtctcactctgttgctcagtccagtcttgaactcctgagctcaagagttccacccgcctcggcctcccaaagttctgggattccaggcgtgagcaccacgcccgacccagctctgctttgttttagtggttctgtttgtgtttgtttctgatgacctctatccaggaatagggttggtttttcttttccatggagtttttaaagagatgatgatttacatggtttaaaactcatgtggactccccgtccatcccagacagcggcttcccacctggaccccatgctcaggattagggctccttccgggtcagaagatgtggcggccccggcctcgggtcaccgtcccctgcacggggtgctcacagcatcttcccctctcctcgttcccagggtgtgcccttactacctgtcccggaacctgaagcagcaagccgacatcgtcttcatgccgtacaattacttgttggatgccaaggtgggggctcagtctgcgtcgctgacaactcctgatgtccaggggggttcctgggcttgggaaaggccgtccgagcctttgctgctgcagggacttagagcagcaggcctgggtgggagagctcacctgtcactgggcaggggctcaacctggccagacacacttgtgagcacccccaggccctgggtcagttgttctgagcaatctgggagtgggcaggctggtgggagtgaggagaggcctccaggctgtggtccacaggccagcgcccactcttgatcctgacagctcaggttctctccttcacgtccgatcacaggaagcaaagcactgagaacacgggaagcccactcactcccctcctcccagccccacactcactcccccccatgcccggccccacactcacttccctcctcgtagcctgtactcacccacatgctgttgtgccccacactttttatttagtgcaggtggcatctcgccgcgggtcagtgacttgtgttcacgtggcgatgacgtggtgacgcttccagatccctccattggtttgctcattctcagggtgtatatttatcaagagcccatcgtgctgggggctgttctaggcatagcaagactggcttcattcatgtggagctttgattctagcggtggggacaggtagacagcagaagagtaagcccgtgagccgatgagactgaagagaagtagagcagagcgaggaggtggagacagagccgagtgggcagggcccaagtgcgatgtcggcaggagatggggaatgctgatgtgtctgaggggagcccgagctggtgcagcagagcaagggaggaggcggggggccgactcctgggcagcgattgtagaaccccacggagagctggtgaggtttgccgtggttgcaggtgactcggtgctttgagccgtggctgcccctgggaaccacctggagagcttctaacccaaccaggcccttccctgggacaattctgtcacagctggtaagctaagtccaacactttcacggaaacacagaagatctaaaacggcacgatgaccgtgaagaacagagctggaggactcacctcactggtttcaagactcctctaaaactgcaggagtggaggtggagacggctcagctcaggcacaagcctgcaggccatggagaaggcagcaagctccagctgacccacacgcttgcggtcattgttttttttttttagttggaatctccctctgtcaccccagttggactgcagtggcaccatctcggctcactgcagcccccacctcctgggttcaagcgattctcccacatcagcctcccgagtcactgggattacaggcgtgcgccaccacgccctgcccgtgatgattgttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttgagacagagtctcgctgtgtcgcccagggtggagtgcagtggccggatctcagctcactgcaagctctgcctcccgggtttttacgccattctcctgcctcagcctcccgagtggccgggactacaggcacccaccacctcgcccggctagttttttgtattttttagtagagacggggtttcaccgtgttagccaggatggtctcgaactcctgacctcgtgatccgcccgtctcggcctcccaaagtgctgggattacaggcttgagccaccgcgcccggccgatgattgttttttgacaaatgcgccaatgtaattgagagaggaaatgaaggttgatttttggttttctgaaaaagtggtgctaagaacagctggatatctgtttggaaaacagcgaatcttaactcttgctttactctatataaacctaaatgtaaaagctaaactaaaggttatagaaagaaacatgggggggtctttgcaactctggggtaggcagagattccttagtgtggatacataaggcactagccgtgaagaaaaacataaaatttggatttcaccaaaatttaaagcttcaacacttgagaagacttgagaaaattaaagagcagttatggaaagggagaaaatgcttctgtctttcagaggacttaaaaaacatttttcagccctcctcatattttaaaggacctttgaccagagtatgtaaaattctctcataactaagcaaaaaacccatttaaccagtgggaaaggatctgcacagacgtttcaccaagatggacggagtggccagttgggcacatggaaggacgcccagcatctccagtcacaggagaaggcagattaaagccacggggagccggcactacggtcccgctagcatgctgaaattcagaagcctgagtgtggcacgaggaggtggaacagctggatctcatccattgctcctaagctgtgaagctgtgtagccactccacaaacacgtgtggcgaacagccgaaccgggagaagggaagacgtgttcaaagatttgtgtgtggccaggctcagtggctcacacctgtaatcccagaactttaggggccaaggctcagggatcgcttacgcccaggagtctgagaccagcctgggcaacatagggagaccccatctcaaaaaaaaagaaaaaagaaaaaaaaagacttaaatgtgcaagtttaccagagctttgtttaagttgccagaactgggaagccgcccacgtgagtccatccactggtgaacggacaaaccgtggttcgcaaaccccaacagcagccgcgggcatgggctgtggtcacacagcagcagggagccagtgagtctcagatgtgctaacccagaggccaactgaggaggacctactgttttttgttttttgatttttgttttgagacagagtcttgctctgtggtgcaggctggagtgcagtggtgtggtctcggctcactgcagcctctgcctctcgggttcaagcagttctcctgcctcagccttccgagtatctgggactacaggcacccgccaccacacccggctaatttttgtatttttagtagagacggcatttcgccatgttggccaggctggtttcaaactcctgaccttgtgatccactcaccttggcctcccaaagtgctgggattacaggcgtgagccaccgcacccagctggacctactgttttattccgtttatatgacgctgtattaatagaagaggcaggggtagagctggtggttatgtggtgcacttaactgccagaactcagtacccttaaaatgggcatcttaatgtgtgaaaacttaaaagtaccaaagctggaccccaccccagcttgctcccatgacactctgtggtgggacctgggagttggggtttttttttttttttttcagatggagtctcactctgttgcccaggctggagtgcagtggtgcaatcttggctcactgcaacctctgcctcccagattaaagcgattctcctgcctcagccttctgagtagctggggttacaggcgcacaccaccacgcccggctaatttttgtgtttttagtagagatggggtttcaccatgttggtcaggctggtctcgaactcctgacctcatgatccacccgcctcggcctcccacagtgctgggattacaggtgggagccaccgcgcccggcctggagttgggtttgtaaatctccctgagtggggccggggcagagaactgctgggtctgggtcttcctggctcctgtggtctgtggctggctgactgcggtggccgggggctcccaggacgtcgtggccgtccgtcttgctgagcgtggcgcgtgcctttccatgctgtgcgtgagcgtctcccggcgtggcgagctgctgcttcgggtggggcggtgttgccaggttgccatccagatctggcctctcctctccacgtcgccggcggtgtttgtcatcggcacttgcgatgttcgatgccggccacatgtgccttggtttccctccttcctgcccctgtgagctccagcaccgtgtcccttctcttcctcctgtagagccgcagagcacacagcattgacctgaaggggacggtcgtgatctttgacgaagctcacaatgtggtgagtctccactgcctcctaaacgcctcctgtttcttcaagagcagcgcaaacctttctggaggggctctggccaaacccttgcagccttcggtgcccaggactgaggactgagcaccccaggagcttctgcaccccttcccactctgatccgatgcctctgctggggctggagactggccagctgggccagggacctgcccctcaggcgcagggcccccacaggccgctccccaggccttccctctagccagctaggggtcagcctgggccaggggtgtctcctctgccctcggcttctgtcaccagggcagcagcaggcgtgtggtctcgcctgcagtgtctctgcccttccggccacatggcctgaggctgaggcagaattgcttgaaccctggaggtggaggctgcggtgagccaggatcacaccgctgcactccagcctgggtgacagagcgggattctgtgtcaaaaaaaaaaaaaatgttgactgggcgcagtaggtcatgcctataatcccagcactttgggaggccgaggcgggcggatcatgaagtcaagagatcaagactatcctggccaacatagtgaaaccccatttctactaaaaatacaaaaatattagctgggcgtggtggtgggtgcctatagtcccagctactcaggaggctgaggcaggagaattgctcgaacccgggaggtggaggttgtagtgagccaagatcacactattgcactctagcctggtgacagcgagactccgtctcaaaaaaaaaaaaaaaaaaaaaattggcaactccattgaaataattgtttgtttgtgttttgagacagggtctcattctgtcgtccaggctggagttcagtggtgtgatcttggtccactgcaacctccacctcctgggctcaagccatcctcctacttcatcctcccaagtaaccaggactacaggtgcacaccaccacgcctggctaatttttgtattttttgtagagatggggttttgcatcattgcccaggctggttttgaactcctgagctcaagtgatctgtccacctcagcctcccaaagtgctgggatcacaggtgtgagccgctgcgcctggcctgaaataatcgtttctcaatattggtgtgggccagacagtcgtgtttggacctgcttgtggcctggccttagagaccccagggcgtggctttggggacttggctgtcagcctcctgtgccttctgcacccaaccccattcctgctttctggaacccccgatcctatcctgctctgtggtgatccgggtgatttgggctcccgatcctatcctgctctgtggtgatccgtgtgattcgggctcctgatcctatcctgctcggtggtgatcccggtgattcgggcccccgatcctatcctgctctgtggtgatccgggtgattcgggctcctgatcctatcccgctctgtggtgatctgggtgattcaggctcccgatcctgtcctgctctgtggtgatccgggtgattcaggctcctgatcctatcctgctctgtggtgatcccggtgattcgggcccccaatcctatcctgctctgtggtgatcccggtgattccggctcccgatcctgtcctgctgtgtggtgatcccggtgattcgggcccccaatcctatcccgctctgtggtggtccgggtgatttgggctcctgatcctgtcctgctctgtggtgatccgtgtgattcaggctcctgatcctgtcctgctcggtggtgatcccagtgattcgggcccccgatcctatcctgctctgtggtgatccgtgtgattcgggctcctgatcctatcctgctctgtggcgatcccggtgatttgggctcccgatcctatcctgctctgtggtgatctgggtgtgctcggactccaggagaagatgtgtgaagaatcagcgtcctttgacctgaccccccatgacctggcttcaggactggacgtcatagaccaggtgctggaggagcagaccaagactgtgcagctgggcgagccccacccggagttcagcacagactcccccagcccaggtgcgttcatagccaggctgctcggtcctgaggcctgcgctgctgcagggggcagcctgccctgtggctgtgtgtggtcggcctgggcactgtctgttcaggctggcactgcagggcacccacttctctcagaggcttctcgggtgtgaattgtttggggtcctgtaggctgtggaagggctggtatcgttcagtagttctggtattttccgaagacctatgtcttctcccagccagtgtcaacttggcctctactgtgtaaaattagagaactatactttgtgaagctgagatgggagcatcgcttgaggccaggagttcgagaccagcctgggcaacatagcgaaacctcgcctctgccaaaaaattagccaggtgtggtggtatggtcctgtggtcctgtggtcagagcttttctggaggctgaagtgggaggagctcttgagcccgggaggcagagcttgccccagatggctccactgtactccagcctgggtgacagagtgaggctgtctccaaaaaaaaaaaaaaaaaaagtaaaactaactatattcactgtaagggcattttgcgtgtttaaatgacccacacatcgggcctgcatcacttgctgtgcccgtagattccttcccagtgttcgtccagaggcgtatttccacacagcgctagtcacggcgtacgtggaaaacgcggaaacccccatggatgtggtcggttggtctgtactgtctgtgttttcttcctttctttttttttttgagatggagttccactcttgttgcccaggctggagtgcaatggtgcgatcttggctcaccgcaacttcctcctcccaggttcaagtgattctcctgcttcagcctcccaagtacctgggactacaggcgcgcgccaccacgcccagctaattttgtatttttagcagagtcagggtttctccatgttggccaggctggtctcaaactccaggtgatccgcccacctcggcctcctgaagtgctgggatccaggcgtgagccgccgcgcccggcctgtgtcgtgttttctgcgtggcctctgtaaacagctgactaggagtctgtgagcatcttcattggttccgctgtgacttgcgtgtgagcatctccagcatcagctttcctcattttcctttaaaatcgcacatgtggatggacaccatggggaccctggaccctgggcagcctcgtcctcacctttctcaccagggtggctgcttgggagagagtgagtttgcgaacttggcatttgttctggctgtaggatccgtttatatatatacatatatttaagtgacagggtctgactctgttgcccaggctggaatgcggtggcgtgatcttagctccctgtagcctcagcctcctgggctcgtggggtcttcccgtctcagcctcttaaagcaccgggaccgcaggcggtcgccactgccatcggatctcaaggctgccacgcagggcgctgcagtaggggaaggggttcctgggactttgaacgctccacctccctcttctccacagtcccccaaccccacctctaactgggagtggatggccacctctttccatcctttgcctggcgcagggtggggagaatgacaggtctccttcccgcatctcggcagctgccctttcgtcggttatatgacgtgggagagaccccggccacccccagggctgtgtgacgtgggagagaccccggccacccccagggctgtgaacaccgccacaggggccttctccactcttcagttttgttagttacgtgacgtgcagggcttttcgttgtaactagtgggggacgtgtggtgaggggggcttctgccgtctcattcaggaccggaacttcagttttcatcgctatttgtcctcctacgcctttggagatggggtctcgctctgttacccaggctggagagcagtggtgccatcagggctcacggcagcctcctcctcctgcagcctccgcctcttgggctcacacaatcctcctgcctcggcctcccaaactcctgggacgactacagacgtgcgccaccgtgcttggcagggttcattcttttcctcacactttatttattggagagcccggtagagcgcccggtttgcacgatctggttgttgagacgttgagaggaaccctgcggagtctgaatctgggtatgggaggggcagtcacacgagttccccggtgtcctcgtctcctccgcacttaggtgcccttaggtggcttgaggaccccagttagactggggccaccctttgcctggctccaggcgggatgtcctttctttttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgggactacaggcgcccgccacctcgcccggctagttttttgtattttttagtagagacggggtttcaccgtgtcagccaggatggtctcgatctcctgacctcgtgatccgcccgcctcggcctcccaaagtgctgggattacaggcttgagccaccgcgcccggccgggatgtcctttctatcaggaggctcacagtatctgctttccagtttcttctgatgttaatgttgttgctgctgaacacctgactctgttgattattgggggattgcaagatggtagtgtcccaattctgtcgcttagtcagttagcaggaataatctagcaccatgctctgcatcgcatcagccgtgtggtgatcccattcacacaggaaaggcgggataaatgctggctgtgggccgggtgtgctgtcccgcatctgtaatcccagcactttgggaggccagggcggtcggatcacgaggtcagagattgagaccatcctggccaacatggtgaaaccccgtctctactaaaaatacaaaaaaaaacccaaaacatgagtgggctgtggtggcgcgtgcttgtaatcccagctaccggggagactgaggcaggagaatcacttgaacccggggggcggaggttgcagagagctgagatcgcgccactgcactccagcctcacaacaaagcgagactctgtctcagtaatcaatcagtcaatcaatcagacgtcatcagtgaatgtacgtgagcgtgtagggattggtcctgctccgcgcgccaccctcaatctcacttctgacctccgttctccacctcgaggtgttttcatgtgggcccgtgtgtcctgaccccagcagttgtggcctctttgctgtctgggagtcaggatttttcacactcatgtcctgctccagacctggaatcagccaagtctccaagaagctctggtttcttttcctgcaaggtggtatttcaggacccacgatgcggcaacaggctggtcatggttactgggttggtcattgttactgggtgtttttgtggagatacatccatacgcacagatgtgttcacaaatgttaactctgaaggtaaaccacccggctgggcacaagggctcaccggtaatcccagcactttgggagaccgagacagttggatgacctgaggtcgggagtttaagaccagcctgggcaacatggtgaaaccccatctctactaaaaacgcaaaaattagccaggtgtggtggcacacgccggtagtcccagctagttgggagacagacatgctggaacctgggacacggaggttgcagagagcagagatagtgccgctgcaccccagcctgggtgatagggtgacaccctgtctccaaaataaatagataaataaagggaaaacgcctcctcttggtgtctccagtttggactggctggcatgggttccggtagcctcttcccccatggtggcgtccttttttccatgccgggaatcctggttctcaagggcggggttgttggtctgagtgtgacacagactgtgcctttgctgcctttctcttgcccagggctgaacatggagctggaagacattgcaaagctgaagagtaagtgctgccctccccaccttcttgcggctgggtggggcctccccctgatgcctcctcaacccgacctgcagtgatcctgctccgcctggagggggccatcgatgccattgagctgcctggagacgacagcggtgtcaccaagccagggaggtgagaggcggggagccagctgcttcactggaggcccagcctggagctagagataggccatggtgcagtcctaggctgtcccaccagtgaggcctgttttcaggcccgttttccctttttgagacctggtaggagcacctggtttgcatgatctggttgttgagacgttgagaggaagagcacacgcatccacggaacagcacacgcatccacggaacagcacacgcatccacggaacagcacacactcccacggaacagcacacgcatccacggaacagcacccactcccacggaacagcacacacacccacggaacagcacatgcttccacggaacagcacatgcttccacggaacagcacacccccacggaacggcacccacggaacagcacgcacgcccccacagaacagcacacacccccacagaacagcacccacgcccccacggaacagcacacacacccacggaacagcacacacgcccccacagaacagcacacacccccacggaacagcacacgcacccatggaacagcacccacggaacagcacacactcccacggaacagcacacgcccccacggaacagcacacacacccacggaacagcacacactcccacggaacagtacacgcatccacggaacagcacacacgcccacggaacagcacacgcatccacggaacagcacacactcccatggaacagcacacgcatccacagaacagcacacacactcccacggaacagcacacaacagcacacactcccacggaacagcacacgcccccacggaacagcacacacacccacggaacagcacacactcccacggaacagcacacgcccccacggaacagcacacacacccacggaacagcacacacgcccccacagaacagcacacacccccacggaacagcacacgcacccatggaacagcacccacggaacagcacacactcccacggaacagcacacgcacccacggaacagcacacacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcacccacggaacagcacgcacgcccccacagaacagcacacacccccacagaacagcacccacgcccccacggaacagcacacacacccacggaacagcacacacgcccccacagaacagcacacacccccacggaacagcacacgcacccatggaacagcacccacggaacagcacacactcccacggaacagcacacgcccccacggaacagcacacactcccacggaacagcacacgcccccacggaacagcacacacacccacggaacagcacacactcccacggaacagtacacgcatccacggaacagcacacacgcccacggaacagcacacgcatccacggaacagcacacactcccatggaacagcacacgcatccacagaacagcacacacactcccacggaacagcacacactcccatggaacagcacacacacacccacggaacagcacacacgcccacggaacagcacacactcccacggaacagcacacacatccacggaacagcacccacggaacagcacccacgcccgcatggaacagcacccacggaacaacacacacccccacggaacagcacacacccccacggaacaacacacactcccacggaacaacacacacccccacggaacaacacacacccccacggaacagcacacacacccacggaacaacacacactcccacggaacaacacacacccccacggaacaacacacacccccacggaacagcacacacacccccacggaacaacacacacccccacggaacagcacacactcccatggaacagcacacacacccacggaacagcacacacacacccacggaacagcacacacacgcccacggaacagcacacacccccacggaacagcacacacccccacggaacagcacacacccccacggaacagcacacacacgcccacggaacagcacacacgcccatgcggggccgctgggtttcctgcagtttgtcctcctccaggcctttccctggacctcctcaggtgcctgttagaatgagaccttcttgttaggacgatgagtgtcccagccaccacctcttttggactccgggaggcccggaacattctgaaggctccgtgggtctcctgtcttctccgcggccagggcagcagcatttgttgtctgtcctgcaggcagatgagggtcaggcctggggcccgtgtggggctccactgagtgcacagccgccaaggcgagcgcgttctgcctgagcgctgaccttgtggcgactcccagttgcttccaggtggagccagtgtcctcagcgtaatgctcaaggctctggcgtggctcctcggccacctgcacccccaggggcccctcctgtagcttctgctgccgccatcactgtcactctcccaaggctttggggactctgcccagagccgctgcctcccagaagcccctgacgaccccccggcgaccccatccctccctctgatgacggcctctgccccgaggcggcttttccttggtgctgttcttgtgccggccaggcctccacttcccatctggaggctcctgagggcggaggcctctcacccccaatgccggcgtcccctggagggctcagtttgtttccgagggaaggaaacttccacagttgctgccttcagttcccaaagctgcagcctgatcccccgtccaggctcgagcctgccttcttctcctcgcagctacatctttgagctgtttgctgaagcccggatcacgtttcagaccaagggctgcatcctggactcgctggaccagatcatccagcacctggccggacgtgagtgccggggcggggtccttggtgtgggcagacgtggcgtagggggtgcagcaggcctccatcctggcagtcagggctcgcctggccatcacctggccgtcagcaggaacaggctcacagaacctcacctggtcggggcgtggaggcgttagtgccggttgcccactgccgtagagcctgtcccagttctgcagctggcggcttcgtcctgcagactcatcccattattccccttttaaggaagagcagcccaaggccctggctgggttgtggggcctcaggcttctccacacaccctgagttctgcttctcagagctgtggggtccagaggctttgcccagaggcagtgtcgtcatgggctgctctggtttgagatgccggcccgccggggcctctgctgtgctcccaggtgctggggagggcggctcttgctgcttcaaaccttaggtgaccatagagcctcttttcaagttccaccggcccccttggagacgctgtccctgcctggctgctctcctggctgctgggaagggcaggcgagctgcccgccctgagtggacactgcgctccaccctgggccccccgttgggcaggagatggagcttggcagtcgggctgagctggcttatggctggaagggcgggggctggggtcggggcctcccctgcctgcagtgcgggtgtcagcgcccggcatgctacctccccaggtgctggagtgttcaccaacacggccggactgcagaagctggcggacatcatccaggtggggcctgctcctctggcgtctccttccctgatggaagccgggcgggtgccttctcctgctgtattagttactgactctagacgtgggggtgggagaacggcccccacaccacctgtttccgactggcaaacgcttggctcctttcctgtgacccaaacccacactgggcctcccgcggggatgggggaggacgaggtctggtggcccgtgcccagggtgacgctggtgggggaggacgtgaaggggagtgggggccagggagccgctcctgccctgtccgggccctcaggccggggggacccactcctggcagccccagctacacgcagatgaagagctctggacacacgtagcttcctgaacagcttctccagggacagacaaacggggaccctgcaggttgccggcgggggtgtccctgcgagcccatgatgggggatgcggccccggcccccttcttggccccgtcctgccctgtgatgcccgtcccctcccatgtgaggttgctcctggctctgtgcctgtggcagcacctgctttccctgaggcagacgggcttgtgcggtttgtggagggcaagccccatttttgggtgctctgggcacacacttgcctctcctggacctcggtgcattttttagaagcagacccacaagtccctgactgggaggccatcagtccttggggaaaccgaggcacaggtggctgtgtggagccatgtgaggctgcctctgtgctgcccacactgaacctcctggccacacgaggtcatggcagggttaggtggggctgcccagctctgtgacaacttcgtgagtgtccgcctggggtggggggtgcttgagctgggggaggccgcagaagaaccctggaatgtaccgccggcctgtgcatgcagtgcggtgctctgtgcatgctactgggcgtggagctgcaggccccaggacgaccaggcactgccttcttcatagacgtggctctgagcaggggcgagacctgggcagggaccaggtggggtgggcacagcaggtggtagggcccaggctcagccctacctccaccgtggccttctctgcggccagtgatgccacagccagtgtcttctctgtgtggcagctggggctggaaagacggcactgctatgtctcccagctcctccccaaaagcacggtgggcatcccagggccagacccctctctctgtggctcctgcctgccaagggctgctttgccgtcccgcctggagcgtggttggctcttcaagcaggaggccgtgcacctgccaggcggacctgcttccatgtcctgacgggtcactcaaagcacctccagcacatggccatcgaggcagccctgcagcccagggcctggagggcaggtgtgagctggcccgggcctgcccctccctggaatgtggcttcccaggctccagtatggagcagtgtccacactctggaaccaaatcttgttcaccccattggtttggaatttcctttggctgctgctctaagtagccactgggagatgactcagctcctcccaaccctggggactgcacaccgtgttcaccctcggctgctcctgagtccctgcaggtgtgggcactgctcatagggctgctgctgggctggacttgcagccctgaggcacaggtgaccctggggacagagtggggctagagtggggctagtggccccaggaagaggatgtgggatgcacggttcagttggaggcaagtccgggcagggccaggctgtgtgctggggaggtagagcttagctggaggcaaactctgggcagtgctggcccgtgctgggcagccagtcaccgctgggcccctgtgagactagtgggtcccctttcttctctgcctgagctgccctggggctgaagcacggggcggtttgtagtggtgtttttttgagacagggtctcgctctgtcaccccagcgggagtgcagggccttgtcaccagctcactgcagcctcagcctcctgggcccaggccatcctcttgcctcagccacctgagtagctgggaacacaggcgtgcaccaccacgcccggctaattttaaaagcttttttgtggagatggggtcttggcgtgcaccaccacgctcggctaattttttaaatttttatgtggagatgggtcttgccatgttgcccaggctggtctcaaagtcctgggctccggcagtctgcccaccttggcctcccacagtgctgggattacaggtaagagcttccaggcctgcccatcagagggctggctttttgagggaagctgtttcctgaggcagcctcagcccctgctcatggcagcccctggtgtcatggtgaatttgttctgttgtttccatccctgtgggccaaatgctttggtaaaacgcaaggccttgtatttagctgtcttgacagtgaaaatggctgggaaggaaggaaggaaggaatggaaggaagtttctctctcccgtgcatacccaggcacgtgcacacgtgcgcagtccacgcacgctgccggtctctgtcccagccgcggtgccggcccccgtgttctccagttcagcttctccgcatttctgtgaacgattccggcttcttgatgttcccagcagaactccctcaagccttcggcttctccagggctctgacggcggctgggctgacgtggccgctctgcggagcctgttggggcttcacgttcctgttctggccatttccggctctttttccgggaagggtcatcacgcgctcttttcctagtgcaggcatcgtccctttcccgttgatttgcagggttctctgtaagatctgaggaacacgtgccaacatattctttcctggtctcgtgctggtcattttttccattttcacgacaggtttggtgaacagatgtttccttgtcatcagacttttgtcttgagcttgtatcccccgaccaagatgctaaactgggtcttgtgtgttctccaaactgcactgtagggtggcggagctttgtgtcggggcctccgcaccttccggcgtcacctgtgggggtgtgaaaggcagactctacctggatttttcccagcacgccacaccggtggttccgtctgctgaccgagtggctcggcttctcccaactccgctgggcacctgccacactgttttcctcacggttttgttcgtggtggttttgtcgtaagtcctggtgtcggccagaaccaatttctgtttttgtttgtttttgagatggagtttcgctcttgtcgcccagggtggagtgcagtggtacgatcttggctcactgtaacctccgcctcctgggttcaaatgattctcctgcctcagcctcctgagtagctgggattacaggtgcttgccaccacgcccagctaattttcatatttttagtagagacggggttttgccgtgtgggccaggctggtctcgaacccctgacctcaggtgatcctctcgcctcagcctcccagagtgctgggattacaggtgtgagccgccgcgcccggcctcttgttcttcttaaacctgccctgatgttttctccatgatgcatgttggagtcaacctgtctgcttcctttaaaaatcttgctgtgattttgactggaatgtgtggaattcctgtgtttttgagtcagtgtctctctgttgcccagtctggagtgcagtgggaccatcacagctcactgcagcctcaacctcctgggctcaggggatcctctcagcccagcctcccaagtagctgggaccacggcacgtgccaccacgcccggctagttttcatatttttttggtggagaccccagggttgcaccatgttgcccaggctggtctcgaactcctgggttcgggcagttcgcccctctcggcctctcagagtgctgggattacaggcgtgagccactgcactaggccatgttgaatttctagattaatttgggacccttaggggcacagagaggagggctgggccagctggtgggaggagaggcctctcgggctgccgcatttccagtgcacggagatggcctgcgtagggggaacagagctcactgggggtccctccagggaggagaaagggtcaggcaggtgccagctcctgtccatcagcctggggctgcatgatggcaggggctggtgaaccgatgacctctgggtgtcctgtgaccttccgtgtgtggggctgatgccgcagaagatcaggtggcctccagggctccgggctctggcctcaggctcctcacgggactgcacctcctctctttcagattgtgtttagtgtggacccctccgagggcagccgtggttccccggcagggctgggacccttacagtcctataaggtaggggccacctccaggaggcaggtggggctgggggtcctgcagtcctgaaaggtaagcggctgcccccaggacacgggcggggtctccacacttctggtcctgtcccctccaggtgcacgtccatcctgatgctggtcaccggaggacggctcagcggtctgatgcctggagcaccactgcagccagaaagcgaggtacagacctgggcccacactctccccgcccgcccgggtgtggtgctcagcaccagcatgccacaggctaggcacgtggccagccgtgggtctcctgcccccctgggcctggccaccttctccatgtccaggccaatccagagcgttctcctcactgtccctctgaaggctggagttactgagagacatgggagagggcctgatggcaccgtgacctgcccagagtcacctggttggtggcggcagagccacagctcagccaggccttcctgctgggacacactggtttatgccaaggccgtcagcacggagccccacagtgaggcacagctttccctgctgcctccacccagcgcctggctgggccaagcctcagggtctcatctgaagggggcccgactggccctgttgtccgaagcctctggtgcgctcggccctgaggccccacgtgccttgttggcttcctgtgctctgtggcgtcttcgagtcggtgctgccgggtacctgtgtggatggggcccgtgagtgggccctcggctccgtgtccagagtcctgtggttcttggagtgtgtccggccccacccggccatgtggtgtccaaggggggctttgtggcggcagcggtgggagctgctgccccttctcgtgctccagtggggccttgcctctgggcttggttcgttcctctctggaacgttctttctcagagctctctggcccgcggtggcacgacgtggccctggctgaagcagcccccgcgtggttgctgtggttggtctgcctggccgagccggaaggggaggcccgggagggcgtcagggttgcgtggcttgacccccggtcggtggctcggtgatggcctcgtggcaaggcgtctcccagcaggaagcgtccatcctggggggtcgtgggaggcggcgcgtgcctgaggcagctggcgcacagatcctgagggcctggaatctatttgttccattgacctgtgacgtcacttgacttctgagaagcagccgctccctgcgttgggtgtttgtaggaaatgagttcctggaggcggaggtcgggaagttcccccgctgcagggcacgctcagccccaggaagcggggtgacctttgctgggagaggtgccttgtccctgctgaccccgagtcacagtcagcgtcgttctgcttgtgccgtgtttaattcctggtgtccggtgcccaggctcagtctcctccccaggccgcggccgctgcggtcagtcagtcaagcacttctaggaacttcctttgaaccggtgcccttgttcgctgctgacgccactcggaggcttcctggcggagcggggaactcgcacttcctcttgtgcgcgtccctggaatgtcgggacgctggcgcttgcttcccgtgcgctcttggcgggatgagcttgccctgagctgcatgtcactgtttctgcagaagtttagggttggagttggctgacctctctggaggtgtccccagcatctgcctggggtctgcctcgtactcccgggacccccagtcccccagaggggaccccagctggcagactgcgctcacactcagggcagcctcctttgttctgacttctgcacgctgggcctgggtggctgtgggcggctcacttgcttgatgccagtgggtgagagggtgatgggcagagaggcaggcggtcaggcccccagtcccgtcctcacaccctgtgtcccctgctcccctccgccccacagggaaggtgttgagctactggtgcttcagtcccggccacagcatgcgcgagctggtccgccagggcgtccgctccctcatccttaccagcggcacgctggccccggtgtcctcctttgccctggagatgcagatgtacgggccacccctgccaggggcctaagcactggtgacgcctgtgacgttagtggggtgggagtgctgggggtccccatgagccggggtgctgggggtcttgggcctcactgggggtctcgggccttgagggctaaaggggtgctggtgtccttccccactgtctgtccctccggccacgctcagccctttcccggtctgcctggagaacccacacatcatcgacaagcaccagatctgggtgggggtcgtccccagaggtcccgacggagcccagctgagctccgcgtttgacagacggtgagggcctgtccctgggccgtgctggggtgcgaggtcggggagtactgagccctgaggccctgagcagtggcctctcggctctgtgcaggttttctgaggagtgcttgtcctccctggggaaggctctgggtaagtgcccggcacaccccagctgtgcccatcctggatcctggaccctgctcccaagagctggtagggaccccggaagacgccccgcccctgccctgaccccagcccctgcacttccaggcaacatcgcccgcgtggtgccctacgggctgctgatcttcttcccttcctatcctgtcatggagaagagcctggagttctggcgagtatgtctcccctgtgtgtcctgggtggggtgggtgagggcagggctggagcacgaagcaggcagtggccacagctcctgcctgccctcatcggatcggcggcgtaaccagggctgccgtgtccctgcctcttcctcccacaggcccgcgacttggccaggaagatggaggcactgaagccgctgtttgtggagcccaggagcaaaggcagcttctccgaggtcagcacttggccggggctctgggtctgctgccccctcgtgcctcccgtgcccctcacagcttccccgaggctgaccaccagccctgaccacgggctctggcggctcccgcctgcctcttcagggcccctgcgtttccttcctggccctgagtgttgcctcttatcttacaaagcccccagcactgggtgggtgtggcgacagtggtcctcctgcctgaggagctatagccggcggccaccctggccctgggattccccgtgttttctgggaagcactgagcaggcgcaggatcagcctgggatccgtgccaggaagaagtttccagaacccagttggccttcctggctaggatagtccttcatcttggaggatgagacctgggtctccctcatgggggaagaaggggccgaggtgggctccaggctgagcctgaccaactttccttccagaccatcagtgcttactatgcaagggtcgctgcccctgggtccaccggcgccaccttcctggcggtgtgccggggcaaggtgagctctctggggccctctgtcctgacccggttgcctgttccccagtgggtgctcatggctcctcagctgactttgggccctgggggctgcctggtcccctccttgggccccacaagagcaaagcactggtggcctgctgggagcgtgccccgctctgggtcgggcaggcaggatgcaggaggttcctggccacgagagttggaggtggcgtctgggagctgtggaccccaagtggggtcctgacccatagatggggcttcctcccacccctggttggagcccccatggccccaggcagctccctggtgtgtcccccaggccagcgaggggttggacttctcagacacgaatggccgtggtgtgatcgtcacgggcctcccgtaccccccccgcatggacccccgggttgtcctcaagatgcagttcctggacgagatgaagggccagggtggggctgggggccaggtgagttacagcagggcggggctggggtaaggcaatctggcgactgagcccccctgtgaccacgggagcccctgtgaccgagcctcctcgccctccagttcctctctgggcaggagtggtaccggcagcaggcgtccagggcggtgaaccaggccatcgggcgagtgatccggcaccgccaggactacggggctgtcttcctctgtgaccacaggtgcgtgcagtccgggggcaggcgtggcgccaggggacacggccacatcccactgggccctggactccttccccacatgaggccccgtctcccccagagcctctctggcgactcggggtcagcctgaggcccctgcggcagatgagggtcttcacttgggtgaactgaccccctgaagcggctgtgggcagggcagcagggctgtggccaccccccaggttcgcctttgctgatgccagagcccagctgccctcctgggtgcgtcctcacgtcagggtgtacgacaacttcggccacgtcatccgagacgtggcccagttcttccgtgttgccgagcgaactgtgagtttctgcccagggaggggatgagggtgttgtccccagaggagccagaaatgggcccacccacccccagtggttctgcagatgccagcaccggccccccgggctgcagcacccagtttgcgtgaaggagaagatgctgtcagggaggccaagtcgcctgaccccctcctctccaccaggaaagctaagagtctggacctgcatgtccccagcctgaagcagaggtcctcaggtgcagatggccagtgcgggacgggcagtgtggggcgggcggggcaggcggattggggcgggccgtgtggggcaagcagcggggcaggtggtgtggggcgggcggcaccaggcgcccagggtggaggcggctcacctggctttatgcactgcccctcccacctccaaaggctgtgtctgcctcctagggtggggttcccacgggctggaaccctcccctacaggcagagaaggcctcaggcaaagacgcgccccaaggctgggactccccctaatagcagggaggacacccacaggcaggggcccccaaatgctgggaccctccctcaggaggggctccgtcataggcagggacccccagctggggccccgcggtgggcttcactgcgcactcgggtactctgcaggatcaccagctgccggggaccccgagagtagcctgtgtgtggagtatgagcaggagccagttcctgcccggcagaggcccagggggctgctggccgccctggagcacagtgaacagcgggccggggaccttggcgaggagcaggtgcagttccagggccttgggatggacacagaccctctgtctcctgaggccaaccccaccccgcccatctgacttcaggcacctccccacacacccctgtaaatcccctgcctgtggggcgggcaggcaggtgagggatcccagctgcctggctgtgtgtgtgccctccacctcgcctcacccacaggctgctggctcccaggtggtgcatgccctggccctcagcgggtgcccccacatcactttgattctctggcaggtcagcttggctcagggcactcaaggtcgggtgcccctgcccctggctgtgcctgaggctggcctttctccaggaatgtgctgcggttgggacccaggttccttcctccttggggccttttgccccagaagcccataatttctcagctttctccctgcttcctgctgggggccgttctcttgctgcctgtccctgcccttcaggctcctggagtgagccccgggtgcaggcaccagacacctgtgtccccttcctgccagcccctcgctgtggtcggactgtccttcctggacctgctcttacgtgtcaccacctgtgagcctcatgagccgctggcgtgacttggacaggaccaagttgtgggactgtcaccagggtgtgccgcgcccctcccccgaccttcgtcttggctactgctcagggctccttggggccatcttccctgtgggcccaggtgctttggggccctggagtgtgtggttggaaccacagctgtgtcctcctcaggcccacagctgctccaccctgtccctcctggctgagaagaggccggcagaggagctgcgaggagggaggaagaagatccggctggtcagccacctggtgcgtgagctgtccctgcacctgtgccgaccaccatagacacgtgtgggaacccggccctgggtgcccccagccaagttgggatgtgggcccgatgggaccagggaagttacccccgggagctgatgtccagggcggctgtgaagctgtcggcccaagggctcaagggcagctgggatcaggccccgcccttgggcaggcttggtttgggggctgcagcactgggtttggccctggcatttccctcaagtgtggacgtgcacctgcctcattcgaggggcgcagcccattcccagccttggatgaaagaaccgagttatagtcggagccaggaagccccctgcctgctggaaagccccaagtgtggcagcctttgtccatgtcccttggcttctgggaagaactgggtggtgcccaggcagggctggtgccatcaggaagtgggcggctgccgaggggcctgggctgttgagggcctgggtggggagtgccggggctgcccctgccttggtttccacgtttccgtgttggtctggggtgtgtggagagatgagcaccgcttcttgggaagcccctccctatgatctgccatcctggagcctcagctgtgtccgctgtggggtgggggctttagggaggaggagagaggacccccagcttgagggagagccagtctaccctttgcccattcgctgccctcggttccctgccagcctctcactgtgtgacccagacaggcccagccccgcagctttcctcccgcagcacctccgtgtccatccagccaaccagtttctcaggcagcagcccccgctcagcaatcactgtcccggggaacacccaatgctccaaggaaggctctgcagctccagggacgggatgatgaggctggccctgatggagcctcgggcctgtgtgctgcaggaggagcccgtggctggcgcacagatggacagggccaagctcttcatggtggctgtgaagcaggaactgagccaagccaactttgccaccttcacccaggccctgcaggactacaagggttccgatgacttcgccgccctggccgcctgtctcggccccctctttgctgaggaccccaagaagcacagcctgctccaaggtgccctggcttgcaggggccgcccatcctgagggcagtgctgccgctgtgtggggtgggggccatctgggtccaaggttgtctctgttctctagagaaaagggggcggatggggacagacaccccttcctctacaggcttctaccagtttgtgcggcctcaccacaaggagcagtttgaggaggtctgtatccagctgacgggacggggctgcggctaccggccagagcacagcgttccccgaaggcagcgggcacagccagccctggaccccactggtaaatggggcctcgggtgggaccctcagactcctccgtggaaggcaccggagtcctgggctggttggggtgggcatccctggggccctgcgtggccccgcctctctgttcccgtgtgggagtgatgggggccaccccccaccaccagcagcagtatcacttgttggggagaccctgtgcaactccacgcagggccctgcccttcccatagcccctgacccctacgcacagccctgtccagctgccgcacgtgtcccctgccccccatgcacagccctggggagtccactgcacacgccagggtcctagacccctagacccctagatccctgtcctcccgtcctccctacttctgcctttgcctagggtggagtccaagcctccagaggtgggagtgtctgtgttcgtgcttgttaacgaacagcccccatagagctcctccagtgtagggttcacccagcggggaacctaacctgttgggaagaccccagaccccttctgggcttggtacccaccctggggctctaagctcctgagcagggctttggcctgcctgccactgttccagcccccatccagcagttggtgtctcctctgctgccccggcacctgggcacgtgtgcctgcctgggttttcccgccctggtccgaggtgggtgaggcctggcctccccagccagcccttccccctccccgggggaactttccaggtgcttgggaccagctttgtggctctccatctcttcatcaggaaggacagcgctagatcccaagctgactgtgtccaaggctgcagcccagcagctggacccccgagagcacctgaaccagggcaggccccacctgtcgcccagaccaccccccacaggtagctgagtcctgaactgtgtgcagcctgtgacttggtgggttcctcagtggcttgatgaggctaacacttgagtgtggcttgagcctttgaaccggaggtgccagtggagctgcccctgtggggagcatctcctggtgggggctcccggttctgtaccccgcagttgtcctgagcagctctccaggagttcgtgggaggaagggcaggcagggtggtgggactctcagtcctctgccccagcgccactctgagccatgctactcccacaccaggaggccctggcagccacccacagcaggggtccagagcgcccagagcagggaagcagggccagcgcgccgtgagcgcctacctggcagatgtccgcagggccctggggtctgcaggctgtagccagctcttggcagcactgacagcctacaagcaagacgatgacctcgacaaggtgctggccgtgctggccgccctgaccactgcaaagccggaggacttccccctgctgcagagcaagtggcctgggcgtgggggacagccagtggggtgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggggcaggggcagggtggggaccgtgccgggtctgagtgaagctccccgcagggttcagcatgtttgtgcgtccacaccacaagcagcgcttctcacagacgtgcacagacctgaccggcagaccctccctgggcacggagccaccaggaccccaggaggagagtcttgccgtgcctcctgtgctcacccacagggctccccaaccaggtagggcgcctgcctggctgctcctgacagtgccccaaccgcacgcagccctgggagtgagcagcaaagccccaggccctgctcagactcagtctccgtctccaggcccctcacggtccgagaagcccggaaagacccagagcgagatctcgaccttccttagacagaggccagcagggactgtgggggcgggcggtgaggctgcagggcccagccagtccccaggacctccccacgggcctgcagcatctgagtggggtgagcctcctgggagagaccttgctgggcagcaggctgcgggagctccgagcgggcccctctcagcaggctgtgtgtgccagggctgtggggcggaggatgtggtgcccttccagtgccctgcctgtgatttccagcgctgccaagcctgttggcaacggcaccttcaggttggtgcctggtcactacagttcctgctgggtgtagccccaggtggtgggctgagggggaaagggccgggtatagccccaggtggtgggctggtggcaacgcctggcaggtgcgtccagcgggccggttgtcttacaggcctctaggacgtgcccatcctgccacaccgcctccaggaagcagagcgtcacgcaggtcttctggccagagccccagtga

SEQ ID NO: 3 - DNA, 18 bp, synthetic construct
  misc_feature 1..18: note = oligonucleotide compound 3_1
  source 1..18: mol_type = other DNA
aattttacatactctggt

SEQ ID NO: 4 - DNA, 19 bp, synthetic construct
  misc_feature 1..19: note = oligonucleotide compound 4_1
  source 1..19: mol_type = other DNA
aattttacatactctggtc

SEQ ID NO: 5 - DNA, 18 bp, synthetic construct
  misc_feature 1..18: note = oligonucleotide compound 5_1
  source 1..18: mol_type = other DNA
ttacatactctggtcaaa

SEQ ID NO: 6 - DNA, 21 bp, synthetic construct
  misc_feature 1..21: note = oligonucleotide compound 6_1
  source 1..21: mol_type = other DNA
ctttattataacttgaatctc

SEQ ID NO: 7 - DNA, 22 bp, Homo sapiens
  source 1..22: mol_type = genomic DNA
tttgaccagagtatgtaaaatt

SEQ ID NO: 8 - DNA, 18 bp, Homo sapiens
  source 1..18: mol_type = genomic DNA
accagagtatgtaaaatt

SEQ ID NO: 9 - DNA, 19 bp, Homo sapiens
  source 1..19: mol_type = genomic DNA
gaccagagtatgtaaaatt

SEQ ID NO: 10 - DNA, 18 bp, Homo sapiens
  source 1..18: mol_type = genomic DNA
tttgaccagagtatgtaa

SEQ ID NO: 11 - DNA, 21 bp, Homo sapiens
  source 1..21: mol_type = genomic DNA
gagattcaagttataataaag

SEQ ID NO: 12 - DNA, 21 bp, synthetic construct
  misc_feature 1..21: note = Primer
  source 1..21: mol_type = other DNA
ggagtgtggattcgcactcct

SEQ ID NO: 13 - DNA, 20 bp, synthetic construct
  misc_feature 1..20: note = Primer
  source 1..20: mol_type = other DNA
agattgagatcttctgcgac

SEQ ID NO: 14 - DNA, 27 bp, synthetic construct
  misc_feature 1..27: note = probe
  source 1..27: mol_type = other DNA
aggcaggtcccctagaagaagaactcc

SEQ ID NO: 15 - DNA, 21 bp, synthetic construct
  misc_feature 1..21: note = primer
  source 1..21: mol_type = other DNA
ccatcctggacattgaggact

SEQ ID NO: 16 - DNA, 21 bp, synthetic construct
  misc_feature 1..21: note = primer
  source 1..21: mol_type = other DNA
caggttccgggacaggtagta

SEQ ID NO: 17 - DNA, 21 bp, synthetic construct
  misc_feature 1..21: note = primer
  source 1..21: mol_type = other DNA
cgtctgtgccttctcatctgc

SEQ ID NO: 18 - DNA, 20 bp, synthetic construct
  misc_feature 1..20: note = primer
  source 1..20: mol_type = other DNA
gcacagcttggaggcttgaa

SEQ ID NO: 19 - DNA, 20 bp, synthetic construct
  misc_feature 1..20: note = primer
  source 1..20: mol_type = other DNA
ccgtctgaactatcctgccc

SEQ ID NO: 20 - DNA, 20 bp, synthetic construct
  misc_feature 1..20: note = primer
  source 1..20: mol_type = other DNA
gccgtagtcggtgtactcgt

SEQ ID NO: 21 - DNA, 19 bp, synthetic construct
  misc_feature 1..19: note = Antisense oligonucleotide
  source 1..19: mol_type = other DNA
gaattttacatactctggt

SEQ ID NO: 22 - DNA, 20 bp, synthetic construct
  misc_feature 1..20: note = Antisense oligonucleotide
  source 1..20: mol_type = other DNA
gaattttacatactctggtc

SEQ ID NO: 23 - DNA, 17 bp, synthetic construct
  misc_feature 1..17: note = Antisense oligonucleotide
  source 1..17: mol_type = other DNA
caaaaaacagtaggtcc

SEQ ID NO: 24 - DNA, 17 bp, synthetic construct
  misc_feature 1..17: note = Antisense oligonucleotide
  source 1..17: mol_type = other DNA
gagggaggtggagcgtt

SEQ ID NO: 25 - DNA, 20 bp, synthetic construct
  misc_feature 1..20: note = Antisense oligonucleotide
  source 1..20: mol_type = other DNA
agctttattataacttgaat

SEQ ID NO: 26 - DNA, 20 bp, synthetic construct
  misc_feature 1..20: note = Antisense oligonucleotide
  source 1..20: mol_type = other DNA
ttttacatactctggtcaaa

SEQ ID NO: 27 - DNA, 18 bp, synthetic construct
  misc_feature 1..18: note = Antisense oligonucleotide
  source 1..18: mol_type = other DNA
ttttacatactctggtca

SEQ ID NO: 28 - DNA, 20 bp, synthetic construct
  misc_feature 1..20: note = Antisense oligonucleotide
  source 1..20: mol_type = other DNA
gagaattttacatactctgg

SEQ ID NO: 29 - DNA, 19 bp, synthetic construct
  misc_feature 1..19: note = Antisense oligonucleotide
  source 1..19: mol_type = other DNA
gcatccaacaagtaattgt

SEQ ID NO: 30 - DNA, 16 bp, synthetic construct
  misc_feature 1..16: note = Antisense oligonucleotide
  source 1..16: mol_type = other DNA
ggtgggtggatgtttc

SEQ ID NO: 31 - DNA, 16 bp, synthetic construct
  misc_feature 1..16: note = Antisense oligonucleotide
  source 1..16: mol_type = other DNA
ggtggtgtggagaagc

SEQ ID NO: 32 - DNA, 18 bp, synthetic construct
  misc_feature 1..18: note = Antisense oligonucleotide
  source 1..18: mol_type = other DNA
ggcatccaacaagtaatt

SEQ ID NO: 33 - DNA, 18 bp, synthetic construct
  misc_feature 1..18: note = Antisense oligonucleotide
  source 1..18: mol_type = other DNA
ggaataaaacagtaggtc

SEQ ID NO: 34 - DNA, 17 bp, synthetic construct
  misc_feature 1..17: note = Antisense oligonucleotide
  source 1..17: mol_type = other DNA
gccattttcactgtcaa

SEQ ID NO: 35 - DNA, 19 bp, synthetic construct
  misc_feature 1..19: note = Antisense oligonucleotide
  source 1..19: mol_type = other DNA
gtgcagaagtcagaacaaa

SEQ ID NO: 36 - DNA, 19 bp, synthetic construct
  misc_feature 1..19: note = Antisense oligonucleotide
  source 1..19: mol_type = other DNA
caaaatgcccttacagtga

SEQ ID NO: 37 - DNA, 17 bp, synthetic construct
  misc_feature 1..17: note = Antisense oligonucleotide
  source 1..17: mol_type = other DNA
ggaataaaacagtaggt

SEQ ID NO: 38 - DNA, 17 bp, synthetic construct
  misc_feature 1..17: note = Antisense oligonucleotide
  source 1..17: mol_type = other DNA
aattttacatactctgg

SEQ ID NO: 39 - DNA, 16 bp, synthetic construct
  misc_feature 1..16: note = oligo
  source 1..16: mol_type = other DNA
aattttacatactctg

SEQ ID NO: 40 - DNA, 17 bp, synthetic construct
  misc_feature 1..17: note = oligo
  source 1..17: mol_type = other DNA
ttacatactctggtcaa

SEQ ID NO: 41 - DNA, 16 bp, synthetic construct
  misc_feature 1..16: note = oligo
  source 1..16: mol_type = other DNA
ttacatactctggtca

SEQ ID NO: 42 - DNA, 15 bp, synthetic construct
  misc_feature 1..15: note = oligo
  source 1..15: mol_type = other DNA
ttacatactctggtc

SEQ ID NO: 43 - DNA, 14 bp, synthetic construct
  misc_feature 1..14: note = Antisense oligonucleotide
  source 1..14: mol_type = other DNA
ttacatactctggt

SEQ ID NO: 44 - DNA, 13 bp, synthetic construct
  misc_feature 1..13: note = Antisense oligonucleotide
  source 1..13: mol_type = other DNA
ttacatactctgg

SEQ ID NO: 45 - DNA, 12 bp, synthetic construct
  misc_feature 1..12: note = Antisense oligonucleotide
  source 1..12: mol_type = other DNA
ttacatactctg

SEQ ID NO: 46 - DNA, 18 bp, synthetic construct
  misc_feature 1..18: note = Antisense oligonucleotide
  source 1..18: mol_type = other DNA
gaattttacatactctgg

SEQ ID NO: 47 - DNA, 17 bp, synthetic construct
  misc_feature 1..17: note = Antisense oligonucleotide
  source 1..17: mol_type = other DNA
gaattttacatactctg

SEQ ID NO: 48 - DNA, 16 bp, synthetic construct
  misc_feature 1..16: note = Antisense oligonucleotide
  source 1..16: mol_type = other DNA
gaattttacatactct

SEQ ID NO: 49 - DNA, 15 bp, synthetic construct
  misc_feature 1..15: note = Antisense oligonucleotide
  source 1..15: mol_type = other DNA
gaattttacatactc

SEQ ID NO: 50 - DNA, 14 bp, synthetic construct
  misc_feature 1..14: note = Antisense oligonucleotide
  source 1..14: mol_type = other DNA
gaattttacatact

SEQ ID NO: 51 - DNA, 13 bp, synthetic construct
  misc_feature 1..13: note = Antisense oligonucleotide
  source 1..13: mol_type = other DNA
gaattttacatac

SEQ ID NO: 52 - DNA, 19 bp, synthetic construct
  misc_feature 1..19: note = Antisense oligonucleotide
  source 1..19: mol_type = other DNA
gaattttacatactctggt

SEQ ID NO: 53 - DNA, 18 bp, synthetic construct
  misc_feature 1..18: note = Antisense oligonucleotide
  source 1..18: mol_type = other DNA
gaattttacatactctgg

SEQ ID NO: 54 - DNA, 17 bp, synthetic construct
  misc_feature 1..17: note = Antisense oligonucleotide
  source 1..17: mol_type = other DNA
gaattttacatactctg

SEQ ID NO: 55 - DNA, 16 bp, synthetic construct
  misc_feature 1..16: note = Antisense oligonucleotide
  source 1..16: mol_type = other DNA
gaattttacatactct

SEQ ID NO: 56 - DNA, 15 bp, synthetic construct
  misc_feature 1..15: note = Antisense oligonucleotide
  source 1..15: mol_type = other DNA
gaattttacatactc

SEQ ID NO: 57 - DNA, 14 bp, synthetic construct
  misc_feature 1..14: note = Antisense oligonucleotide
  source 1..14: mol_type = other DNA
gaattttacatact

SEQ ID NO: 58 - DNA, 16 bp, synthetic construct
  misc_feature 1..16: note = Antisense oligonucleotide
  source 1..16: mol_type = other DNA
caaaaaacagtaggtc

SEQ ID NO: 59 - DNA, 15 bp, synthetic construct
  misc_feature 1..15: note = Antisense oligonucleotide
  source 1..15: mol_type = other DNA
caaaaaacagtaggt

SEQ ID NO: 60 - DNA, 14 bp, synthetic construct
  misc_feature 1..14: note = Antisense oligonucleotide
  source 1..14: mol_type = other DNA
caaaaaacagtagg

SEQ ID NO: 61 - DNA, 13 bp, synthetic construct
  misc_feature 1..13: note = Antisense oligonucleotide
  source 1..13: mol_type = other DNA
caaaaaacagtag

SEQ ID NO: 62 - DNA, 12 bp, synthetic construct
  misc_feature 1..12: note = Antisense oligonucleotide
  source 1..12: mol_type = other DNA
caaaaaacagta

SEQ ID NO: 63 - DNA, 11 bp, synthetic construct
  misc_feature 1..11: note = Antisense oligonucleotide
  source 1..11: mol_type = other DNA
caaaaaacagt

SEQ ID NO: 64 - DNA, 16 bp, synthetic construct
  misc_feature 1..16: note = Antisense oligonucleotide
  source 1..16: mol_type = other DNA
gagggaggtggagcgt

SEQ ID NO: 65 - DNA, 15 bp, synthetic construct
  misc_feature 1..15: note = Antisense oligonucleotide
  source 1..15: mol_type = other DNA
gagggaggtggagcg

SEQ ID NO: 66 - DNA, 14 bp, synthetic construct
  misc_feature 1..14: note = Antisense oligonucleotide
  source 1..14: mol_type = other DNA
gagggaggtggagc

SEQ ID NO: 67 - DNA, 13 bp, synthetic construct
  misc_feature 1..13: note = Antisense oligonucleotide
  source 1..13: mol_type = other DNA
gagggaggtggag

SEQ ID NO: 68 - DNA, 13 bp, synthetic construct
  misc_feature 1..13: note = Antisense oligonucleotide
  source 1..13: mol_type = other DNA
gagggaggtggag

SEQ ID NO: 69 - DNA, 11 bp, synthetic construct
  misc_feature 1..11: note = Antisense oligonucleotide
  source 1..11: mol_type = other DNA
gagggaggtgg

SEQ ID NO: 70 - DNA, 24 bp, synthetic construct
  misc_feature 1..24: note = Primer
  source 1..24: mol_type = other DNA
gtttttcacctctgcctaatcatc

SEQ ID NO: 71 - DNA, 23 bp, synthetic construct
  misc_feature 1..23: note = Primer
  source 1..23: mol_type = other DNA
gcaaaaagttgcatggtgctggt